SEQUENCE LISTING (90 sequences)

SEQ ID NO: 1 - protein, 330 aa, Homo sapiens [sequence omitted]

SEQ ID NO: 2 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 3 - protein, 15 aa, Homo sapiens
EPKSCDKTHTCPPCP

SEQ ID NO: 4 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 5 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 8 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 107 aa, Homo sapiens
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 10 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 11 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, synthetic construct
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 107 aa, synthetic construct
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 107 aa, synthetic construct
GQPREPQVYTLPPSREEMTKNQVSLWCLVKGFYPSDISVEWESNGQPENNYKTTPPMLDSDGSFFLYSKLTVDKSRWQQGNVFSCSVMHEALHNHYTQKSLSLSPGK

SEQ ID NO: 15 - protein, 107 aa, synthetic construct
GQPREPQVYTLPPSREEMTKNQVSLWCLVKGFYPSDIAVEWESSGQPENNYNTTPPMLDSDGSFFLYSKLTVDKSRWQQGNIFSCSVMHEALHNRFTQKSLSLSPGK

SEQ ID NO: 16 - protein, 107 aa, synthetic construct
GQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 107 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 107 aa, synthetic construct
GQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 107 aa, synthetic construct
GQPREPQVYTLPPCREEMTKNQVSLWCLVKGFYPSDISVEWESNGQPENNYKTTPPMLDSDGSFFLYSKLTVDKSRWQQGNVFSCSVMHEALHNHYTQKSLSLSPGK

SEQ ID NO: 20 - protein, 107 aa, synthetic construct
GQPREPQVYTLPPCREEMTKNQVSLWCLVKGFYPSDIAVEWESSGQPENNYNTTPPMLDSDGSFFLYSKLTVDKSRWQQGNIFSCSVMHEALHNRFTQKSLSLSPGK

SEQ ID NO: 21 - protein, 107 aa, synthetic construct
GQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 107 aa, synthetic construct
GQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 107 aa, synthetic construct
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 107 aa, synthetic construct
GQPREPQVYTLPPSREEMTKNQVSLSCAVKGFYPSDISVEWESNGQPENNYKTTPPMLDSDGSFFLVSKLTVDKSRWQQGNVFSCSVMHEALHNHYTQKSLSLSPGK

SEQ ID NO: 25 - protein, 107 aa, synthetic construct
GQPREPQVYTLPPSREEMTKNQVSLSCAVKGFYPSDIAVEWESSGQPENNYNTTPPMLDSDGSFFLVSKLTVDKSRWQQGNIFSCSVMHEALHNRFTQKSLSLSPGK

SEQ ID NO: 26 - protein, 107 aa, synthetic construct
GQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 27 - protein, 107 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 28 - protein, 107 aa, synthetic construct
GQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 107 aa, synthetic construct
GQPREPQVCTLPPSREEMTKNQVSLSCAVKGFYPSDISVEWESNGQPENNYKTTPPMLDSDGSFFLVSKLTVDKSRWQQGNVFSCSVMHEALHNHYTQKSLSLSPGK

SEQ ID NO: 30 - protein, 107 aa, synthetic construct
GQPREPQVCTLPPSREEMTKNQVSLSCAVKGFYPSDIAVEWESSGQPENNYNTTPPMLDSDGSFFLVSKLTVDKSRWQQGNIFSCSVMHEALHNRFTQKSLSLSPGK

SEQ ID NO: 31 - protein, 107 aa, synthetic construct
GQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 32 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 33 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 34 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 35 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 216 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLWCLVKGFYPSDISVEWESNGQPENNYKTTPPMLDSDGSFFLYSKLTVDKSRWQQGNVFSCSVMHEALHNHYTQKSLSLSPGK

SEQ ID NO: 40 - protein, 217 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLWCLVKGFYPSDIAVEWESSGQPENNYNTTPPMLDSDGSFFLYSKLTVDKSRWQQGNIFSCSVMHEALHNRFTQKSLSLSPGK

SEQ ID NO: 41 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 216 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPCREEMTKNQVSLWCLVKGFYPSDISVEWESNGQPENNYKTTPPMLDSDGSFFLYSKLTVDKSRWQQGNVFSCSVMHEALHNHYTQKSLSLSPGK

SEQ ID NO: 45 - protein, 217 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPCREEMTKNQVSLWCLVKGFYPSDIAVEWESSGQPENNYNTTPPMLDSDGSFFLYSKLTVDKSRWQQGNIFSCSVMHEALHNRFTQKSLSLSPGK

SEQ ID NO: 46 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 107 aa, synthetic construct; identical to SEQ ID NO: 23

SEQ ID NO: 49 - protein, 216 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLSCAVKGFYPSDISVEWESNGQPENNYKTTPPMLDSDGSFFLVSKLTVDKSRWQQGNVFSCSVMHEALHNHYTQKSLSLSPGK

SEQ ID NO: 50 - protein, 217 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLSCAVKGFYPSDIAVEWESSGQPENNYNTTPPMLDSDGSFFLVSKLTVDKSRWQQGNIFSCSVMHEALHNRFTQKSLSLSPGK

SEQ ID NO: 51 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 52 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 53 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 216 aa, synthetic construct
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDISVEWESNGQPENNYKTTPPMLDSDGSFFLVSKLTVDKSRWQQGNVFSCSVMHEALHNHYTQKSLSLSPGK

SEQ ID NO: 55 - protein, 217 aa, synthetic construct
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVCTLPPSREEMTKNQVSLSCAVKGFYPSDIAVEWESSGQPENNYNTTPPMLDSDGSFFLVSKLTVDKSRWQQGNIFSCSVMHEALHNRFTQKSLSLSPGK

SEQ ID NO: 56 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 57 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 58 - protein, 184 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 59 - protein, 170 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 60 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 61 - protein, 203 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 62 - protein, 178 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 63 - protein, 164 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 64 - protein, 169 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 65 - protein, 197 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 66 - protein, 5 aa, synthetic construct
GGGGS

SEQ ID NO: 67 - protein, 10 aa, synthetic construct
GGGGSGGGGS

SEQ ID NO: 68 - protein, 15 aa, synthetic construct
GGGGSGGGGSGGGGS

SEQ ID NO: 69 - protein, 20 aa, synthetic construct
GGGGSGGGGSGGGGSGGGGS

SEQ ID NO: 70 - protein, 378 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 71 - protein, 350 aa, synthetic construct
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 72 - protein, 360 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 73 - protein, 416 aa, synthetic construct
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 74 - protein, 366 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 75 - protein, 338 aa, synthetic construct
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 76 - protein, 348 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 77 - protein, 404 aa, synthetic construct
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 78 - protein, 483 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 708 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 80 - protein, 291 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 81 - protein, 103 aa, synthetic construct
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 82 - protein, 107 aa, synthetic construct
RTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 4 aa, synthetic construct
  REGION >4: note = The entire sequence of amino acids 1-4 can be repeated two to six times
GGGS

SEQ ID NO: 84 - protein, 5 aa, synthetic construct
  REGION >5: note = The entire sequence of amino acids 1-5 can be repeated two to six times
GGGGS

SEQ ID NO: 85 - protein, 5 aa, synthetic construct
  REGION 1..4: note = The amino acid residues 1-4 can be repeated two to six times
GGGSG

SEQ ID NO: 86 - protein, 6 aa, synthetic construct
  REGION 1..5: note = The amino acid residues 1-5 can be repeated two to six times
GGGGSG

SEQ ID NO: 87 - protein, 6 aa, synthetic construct
  REGION 1..4: note = The amino acid residues 1-4 can be repeated two to six times
GGGSGG

SEQ ID NO: 88 - protein, 7 aa, synthetic construct
  REGION 1..4: note = The amino acid residues 1-4 can be repeated two to six times
GGGSGGG

SEQ ID NO: 89 - protein, 7 aa, synthetic construct
  REGION 1..5: note = The amino acid residues 1-5 can be repeated two to six times
GGGGSGG

SEQ ID NO: 90 - protein, 8 aa, synthetic construct
  REGION 1..5: note = The amino acid residues 1-5 can be repeated two to six times
GGGGSGGG

(19) United States
(12) Patent Application Publication
(10) Pub. No.: US 2026/0193325 A1
(43) Pub. Date: Jul. 9, 2026

(54) METHODS FOR PRODUCING MOLECULES

(71) Applicant: Hoffmann-La Roche Inc., Little Falls, NJ (US)

(72) Inventors: Timo FRENSING, Remscheid (DE); Jasmin HUETTEN, Peissenberg (DE); Oliver POPP, Penzberg (DE)

(73) Assignee: Hoffmann-La Roche Inc., Little Falls, NJ (US)

(21) Appl. No.: 19/578,149

(22) Filed: Mar. 25, 2026

Related U.S. Application Data
(63) Continuation of application No. PCT/EP2024/076567, filed on Sep. 23, 2024.

(30) Foreign Application Priority Data
Sep. 25, 2023 (EP) 23199319.7

(51) Int. Cl. C07K 16/00

(52) U.S. Cl. CPC C07K 16/00; C07K 2317/31; C07K 2317/522; C07K 2317/526; C07K 2317/53; C07K 2317/55; C07K 2319/00

(57) ABSTRACT

Methods are disclosed for producing a molecule comprising a polypeptide complex formed by interaction between a first polypeptide comprising a CH3 region comprising a knob modification and a second polypeptide comprising a CH3 region comprising a hole modification.

### CROSS REFERENCE TO RELATED APPLICATIONS

This application is a continuation of International Application No. PCT/EP2024/076567, filed Sep. 23, 2024, which claims priority to EP Application No 23199319.7, filed Sep. 25, 2023, each of which is incorporated herein by reference in its entirety.

### SEQUENCE LISTING

This application contains a Sequence Listing which has been submitted electronically in XML format and is hereby incorporated by reference in its entirety. Said XML copy, created on Mar. 23, 2026, is named “P38323-US-1_Sequence_Listing.xml” and is 96,494 bytes in size.

### TECHNICAL FIELD

The present disclosure relates to the fields of molecular biology and target-binding molecule production technology.

### BACKGROUND

Recombinant co-expression of constituent polypeptides of target-binding molecules and their subsequent association leads to several possible combinations. To improve the yield of the desired combinations of polypeptides in target-binding molecules in recombinant production, it is advantageous to introduce Fc region modification(s) promoting association of the desired combinations of polypeptides. Modifications may promote e.g. hydrophobic and/or electrostatic interaction between CH2 and/or CH3 regions of different polypeptide chains. Such modifications are described e.g. in Ha et al., Front Immunol. (2016) 7:394, which is hereby incorporated by reference in its entirety.

One such modification is the ‘knobs-into-holes’ or ‘KiH’ modification which is described e.g. in U.S. Pat. No. 7,695,936, Atwell et al., J Mol Biol. (1997) 270(1):26-35 and Carter, J Immunol Meth. (2001) 248(1-2):7-15. In such molecules, one of the CH3 regions of the Fc region comprises a ‘knob’ modification, and the other CH3 region comprises a ‘hole’ modification. The ‘knob’ and ‘hole’ modifications are positioned within the respective CH3 regions so that the ‘knob’ can be positioned in the ‘hole’ in order to promote heterodimerisation (and inhibit homodimerisation) of the polypeptides and/or stabilise heterodimers. Knobs are constructed by substituting amino acids having small chains with those having larger side chains (e.g. tyrosine or tryptophan). Holes are created by substituting amino acids having large side chains with those having smaller side chains (e.g. alanine or threonine). Atwell et al., J Mol Biol. (1997) 270(1):26-35 describes the traditional ‘knob’ modification T366W, and ‘hole’ modification Y407V:T366S:L368A. KiH modifications comprising additional substitutions have also been described, e.g. for the formation of stabilising interchain disulfide bonds (see Merchant et al., Nature Biotech. (1998) 16:677-681).

Notwithstanding such modification to promote the desired pairing of CH3 region-bearing polypeptides, undesired molecules such as mispaired, hole-hole homodimers are typically present in the products of cell culture for the production of KiH molecules, in significant proportions. Chen et al., Bioresources and Bioprocessing (2022) 9:72 describes an approach for the efficient removal of such undesired side products. There remains a need in the art to increase the production of the desired KiH molecules relative to the production of undesired side products such as mispaired, hole-hole homodimers.

### SUMMARY

In a first aspect, the present disclosure provides a method for producing a molecule comprising a polypeptide complex formed by interaction between a first polypeptide comprising a CH3 region comprising a knob modification and a second polypeptide comprising a CH3 region comprising a hole modification, wherein the method comprises culturing cells comprising nucleic acid encoding the first and second polypeptides for the majority of the period of culture in cell culture medium comprising cysteine at a concentration of less than 6 mM.

In some embodiments, the cell culture medium has an osmolality greater than 300 mOsmol/kg.

In some embodiments, the method comprises seeding the cells in cell culture medium comprising cysteine at a concentration of less than 6 mM, optionally wherein the method comprises seeding the cells in cell culture medium comprising cysteine at a concentration of from 1 mM to 5 mM.

In some embodiments, the method comprises seeding the cells in cell culture medium comprising cysteine at a concentration of from 2.5 mM to 4.75 mM.

In some embodiments, the method comprises seeding the cells in cell culture medium having an osmolality greater than 300 mOsmol/kg, optionally wherein the method comprises seeding the cells in cell culture medium having an osmolality of from 320 mOsmol/kg to 420 mOsmol/kg.

In some embodiments, the method comprises seeding the cells in cell culture medium having an osmolality of about 370 mOsmol/kg.

In some embodiments, the period of culture is at least 3 days, optionally wherein the period of culture is at least 7 days or at least 14 days.

In some embodiments, the cell culture medium has a pH of from 6.8 to 7.4, optionally wherein the cell culture medium has a pH of about 7.2.

In some embodiments, the first polypeptide or the second polypeptide comprises an amino acid sequence having at least 70% amino acid sequence identity to the amino acid sequence of the extracellular domain of a ligand for a costimulatory molecule.

In some embodiments:
(i) the first polypeptide comprises an amino acid sequence having at least 70% amino acid sequence identity to the amino acid sequence of the extracellular domain of a ligand for a costimulatory molecule, and the second polypeptide comprises an amino acid sequence forming all or part of an antigen-binding moiety that binds to a target antigen; or
(ii) the first polypeptide comprises an amino acid sequence forming all or part of an antigen-binding moiety that binds to a target antigen, and the second polypeptide comprises an amino acid sequence having at least 70% amino acid sequence identity to the amino acid sequence of the extracellular domain of a ligand for a costimulatory molecule.

### DESCRIPTION

### Molecules Produced Comprising a Polypeptide Complex Formed by Interaction Between a First Polypeptide Comprising a CH3 Region Comprising a Knob Modification and a Second Polypeptide Comprising a CH3 Region Comprising a Hole Modification

Aspects and embodiments of the present disclosure relate to the production of molecules comprising a polypeptide complex formed by interaction between a first polypeptide comprising a CH3 region comprising a knob modification and a second polypeptide comprising a CH3 region comprising a hole modification.

Herein, ‘a molecule comprising a polypeptide complex formed by interaction between a first polypeptide comprising a CH3 region comprising a knob modification and a second polypeptide comprising a CH3 region comprising a hole modification’ may for conciseness be referred to simply as ‘a KiH molecule’. Similarly, ‘molecules comprising a polypeptide complex formed by interaction between a first polypeptide comprising a CH3 region comprising a knob modification and a second polypeptide comprising a CH3 region comprising a hole modification’ may be referred to simply as ‘KiH molecules’.

As used herein, a ‘peptide’ refers to a chain of two or more amino acid monomers linked by peptide bonds. Peptides typically have a length in the region of about 2 to about 50 amino acids.

A ‘polypeptide’ comprises more than one peptide bond, and comprises 3 or more amino acids. ‘Polypeptide complexes’ are characterised by protein:protein interaction between their constituent polypeptides. In some embodiments, protein:protein interaction comprises non-covalent interaction, e.g. electrostatic interaction (e.g. ionic bonding, hydrogen bonding) and/or Van der Waals forces. In some embodiments, protein:protein interaction comprises covalent interaction (e.g. disulfide bonding, electron sharing). By way of illustration polypeptide complexes contemplated in accordance with the present disclosure include molecules comprising polypeptide complexes formed by interaction between the CH3 regions of a first polypeptide comprising a CH3 region and a second polypeptide comprising a CH3 region.

Immunoglobulins and their structures are described e.g. in Schroeder and Cavacini J Allergy Clin Immunol. (2010) 125(202):S41-S52, which is hereby incorporated by reference in its entirety. Immunoglobulins of type G (i.e. IgG) are ~150 kDa glycoproteins comprising two heavy chains and two light chains. From N- to C-terminus, immunoglobulin heavy chains comprise a heavy chain variable region (VH) followed by a heavy chain constant region comprising three constant regions (CH1, CH2, and CH3, with a CH1-CH2 hinge region provided between CH1 and CH2). Immunoglobulin light chains comprise a light chain variable region (VL) followed by a light chain constant region (CL). Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

A ‘CH1 region’ refers to an amino acid sequence corresponding to the CH1 region of an immunoglobulin. The CH1 region is the region of an Ig formed by positions 118 to 215 of the Ig constant region, according to the EU numbering system described in Edelman et al., Proc Natl Acad Sci USA. (1969) 63(1):78-85. A ‘CH1-CH2 hinge region’ refers to an amino acid sequence corresponding to the CH1-CH2 hinge region of an immunoglobulin. The CH1-CH2 hinge region is the region of an Ig formed by positions 216 to 230 of the Ig constant region, according to the EU numbering system described in Edelman et al., Proc Natl Acad Sci USA. (1969) 63(1):78-85. A ‘CH2 region’ refers to an amino acid sequence corresponding to the CH2 region of an immunoglobulin. The CH2 region is the region of an Ig formed by positions 231 to 340 of the Ig constant region, according to the EU numbering system described in Edelman et al., Proc Natl Acad Sci USA. (1969) 63(1):78-85. A ‘CH2-CH3 region’ refers to an amino acid sequence corresponding to the CH2 and CH3 regions of an immunoglobulin. The CH2-CH3 region is the region of an Ig formed by positions 231 to 447 of the Ig constant region, according to the EU numbering system described in Edelman et al., Proc Natl Acad Sci USA. (1969) 63(1):78-85.

A ‘CH3 region’ refers to an amino acid sequence corresponding to the CH3 region of an immunoglobulin. The CH3 region is the region of an Ig formed by positions 341 to 447 of the Ig constant region, according to the EU numbering system described in Edelman et al., Proc Natl Acad Sci USA. (1969) 63(1):78-85. The CH3 region of human IgG1 (G1m1 allotype) is shown in SEQ ID NO:5. The CH3 region of human IgG1 (G1m3 allotype) is shown in SEQ ID NO:8.

In some embodiments, a CH3 region comprises, or consists of, an amino acid sequence having the amino acid sequence of SEQ ID NO:5, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:5. In some embodiments, a CH3 region comprises, or consists of, an amino acid sequence having the amino acid sequence of SEQ ID NO:8, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:8.

CH3 regions according to the present disclosure comprise modification to promote association with another CH3 region. In some embodiments, a CH3 region comprises modification to promote heteromerisation, i.e. association between non-identical CH3 regions.

In embodiments herein (and also in the art more generally), a ‘modification’ may also be referred to as a ‘substitution’ or a ‘mutation’.

In aspects and embodiments of the present disclosure, interaction between CH3 regions of constituent polypeptides of molecules described herein is potentiated through ‘knob-into-hole’ technology. Knob-into-hole (or ‘KiH’) technology is described e.g. in WO 96/027011, Ridgway, J. B., et al., Protein Eng. (1996) 9(7):617-621, Merchant, A. M., et al., Nat Biotechnol. (1998) 16(7):677-681, U.S. Pat. No. 7,695,936 and Carter, J Immunol Meth. (2001) 248(1-2):7-15, all of which are hereby incorporated by reference in their entirety.

Heterodimerisation between CH3 region-bearing polypeptides is promoted through modification of their interaction surfaces to provide complementary ‘knob’ and ‘hole’ modifications in the amino acid sequences of the CH3 regions of the polypeptides. The ‘knob’ and ‘hole’ modifications are positioned within the respective CH3 regions so that the ‘knob’ can be positioned in the ‘hole’ in order to promote heterodimerisation (and inhibit homodimerisation) of the polypeptides, and/or stabilise heterodimers. Knobs are constructed by substituting amino acids having small side chains with those having larger side chains (e.g. tyrosine or tryptophan). Holes are created by substituting amino acids having large side chains with those having smaller side chains (e.g. valine, alanine, serine or threonine).

By way of illustration, in embodiments herein, certain CH3 regions comprise a knob modification, which may comprise modification at position 366 of the CH3 region (unless otherwise stated, numbering of positions or substitutions in CH3 regions herein is according to the EU numbering system described in Edelman et al., Proc Natl Acad Sci USA. (1969) 63(1):78-85). The modification may be to provide a tryptophan residue at position 366, which in the canonical sequences for human IgG1/IgG2/IgG3/IgG4 is a threonine residue.

In embodiments and aspects of the present disclosure, CH3 regions—e.g. CH3 regions of constituent polypeptides of polypeptide complexes according to the present disclosure—comprise paired CH3 region ‘KiH’ or ‘KiH_{S-S}’ modifications.

In some embodiments, a CH3 region comprising a knob modification comprises a tryptophan residue at position 366 (i.e. 366W). In some embodiments, a CH3 region comprising a knob modification comprises a tyrosine residue at position (i.e. 366Y). In some embodiments, the knob modification is or comprises T366W or T366Y. In some embodiments, a CH3 region comprising a knob modification comprises 366W. In some embodiments, the knob modification is or comprises T366W.

Herein, when reference is made to a position of an immunoglobulin constant region (e.g. according to EU numbering), the corresponding position in homologous sequences to the constant region sequence of human IgG1 (G1m1 allotype) are also contemplated. Corresponding positions to those identified in the CH3 region of human IgG1 (G1m1 allotype) can be identified by sequence alignment, which can be performed e.g. using sequence alignment software such as ClustalOmega (Söding, J. Bioinformatics (2005) 21:951-960). By way of illustration, 366T in human IgG1 CH3 region (position 26 of SEQ ID NO:5) corresponds to position 26 of SEQ ID NO:8 (hIgG1 G1m3 allotype CH3 region), position 26 of SEQ ID NO:9 (hIgG2 CH3 region), position 26 of SEQ ID NO:10 (hIgG3 CH3 region), and position 26 of SEQ ID NO:11 (hIgG4 CH3 region).

In some embodiments, a CH3 region comprising a hole modification comprises a serine, valine or alanine at position 366 (i.e. 366S, 366V, or 366A). In some embodiments, a CH3 region comprising a hole modification comprises an alanine, valine, serine or threonine residue at position 368 (i.e. 368A, 368V, 368S or 368T). In some embodiments, a CH3 region comprising a hole modification comprises a valine, alanine, serine or threonine residue at position 407 (i.e. 407V, 407A, 407S or 407T). In some embodiments, a CH3 region comprising a hole modification comprises: 366S, 366V or 366A; 368A, 368V, 368S or 368T; and 407V, 407A, 407S or 407T. In some embodiments, the hole modification is, or comprises, T366S, T366V or T366A; L368A, L368V, L368S or L368T; and Y407V, Y407A, Y407S or Y407T.

In some embodiments, a CH3 region comprising a hole modification comprises 366S. In some embodiments, a CH3 region comprising a hole modification comprises 368A. In some embodiments, a CH3 region comprising a hole modification comprises 407V. In some embodiments, a CH3 region comprising a hole modification comprises: 366S, 368A and 407V. In some embodiments, the hole modification is, or comprises, T366S, L368A and Y407V.

In some embodiments, a CH3 region comprising a hole modification comprises: 366S, 368A and 407V. In some embodiments, the hole modification is, or comprises, Y407V, Y407A, Y407S or Y407T; T366S, T366V or T366A; and L368A, L368V, L368S or L368T.

In some embodiments, a CH3 region comprising a hole modification comprises 407V, 366S and 368A. In some embodiments, the hole modification is or comprises Y407V, T366S, and L368A.

An additional interchain disulfide bridge between the CH3 regions may also be introduced (as described in Merchant, A. M., et al., Nature Biotech. (1998) 16:677-681), e.g. through introduction of a cysteine residue at position 354 of the CH3 region having a ‘knob’ modification, and introduction of a cysteine residue at position 349 of the CH3 region having a ‘hole’ modification. Knob modifications further comprising modification to introduce a cysteine residue for the formation of an interchain disulfide bridge may be referred to as ‘knob-cys’ modifications, and similarly hole modifications further comprising modification to introduce a cysteine residue for the formation of an interchain disulfide bridge may be referred to as ‘hole-cys’ modifications.

Accordingly, in some embodiments, a CH3 region comprising a knob modification comprises 354C. In some embodiments, the knob modification comprises S354C. In some embodiments, a CH3 region comprising a knob modification comprises 366W or 366Y; and 354C. In some embodiments, a CH3 region comprising a knob modification comprises 366W and 354C. In some embodiments, a knob modification is, or comprises, T366W or T366Y; and S354C. In some embodiments, a knob modification is, or comprises, T366W and S354C.

Knob modifications according to the preceding paragraph may be referred to as ‘knob-cys’ modifications.

Accordingly, in some embodiments, a CH3 region comprising a hole modification comprises 349C. In some embodiments, the hole modification comprises Y349C. In some embodiments, a CH3 region comprising a hole modification comprises Y407V, Y407A, Y407S or Y407T; T366S, T366V or T366A; L368A, L368V, L368S or L368T; and Y349C. In some embodiments, a CH3 region comprising a hole modification comprises 366S, 368A, 407V and Y349C.

Hole modifications according to the preceding paragraph may be referred to as ‘hole-cys’ modifications.

In some embodiments, a knob modification is, or comprises, T366W or T366Y; and S354C. In some embodiments, a hole modification is, or comprises, T366S, L368A, Y407V and Y349C.

In some embodiments, a CH3 region comprising a knob modification comprises, or consists of:
an amino acid sequence having the amino acid sequence of SEQ ID NO:12, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:12; or
an amino acid sequence having the amino acid sequence of SEQ ID NO:13, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:13; or
an amino acid sequence having the amino acid sequence of SEQ ID NO:14, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:14; or
an amino acid sequence having the amino acid sequence of SEQ ID NO:15, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:15; or
an amino acid sequence having the amino acid sequence of SEQ ID NO:16, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:16; or
an amino acid sequence having the amino acid sequence of SEQ ID NO:17, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:17; or
an amino acid sequence having the amino acid sequence of SEQ ID NO:18, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:18; or
an amino acid sequence having the amino acid sequence of SEQ ID NO:19, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:19; or
an amino acid sequence having the amino acid sequence of SEQ ID NO:20, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:20; or
an amino acid sequence having the amino acid sequence of SEQ ID NO:21, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:21;
wherein the amino acid sequence comprises 366W.

In some embodiments, a CH3 region comprising a hole modification comprises, or consists of:
an amino acid sequence having the amino acid sequence of SEQ ID NO:22, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:22; or
an amino acid sequence having the amino acid sequence of SEQ ID NO:23, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:23; or
an amino acid sequence having the amino acid sequence of SEQ ID NO:24, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:24; or
an amino acid sequence having the amino acid sequence of SEQ ID NO:25, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:25; or
an amino acid sequence having the amino acid sequence of SEQ ID NO:26, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:26; or
an amino acid sequence having the amino acid sequence of SEQ ID NO:27, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:27; or
an amino acid sequence having the amino acid sequence of SEQ ID NO:28, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:28; or
an amino acid sequence having the amino acid sequence of SEQ ID NO:29, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:29; or
an amino acid sequence having the amino acid sequence of SEQ ID NO:30, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:30; or
an amino acid sequence having the amino acid sequence of SEQ ID NO:31, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:31;
wherein the amino acid sequence comprises 366S, 368A and 407V.

In some embodiments, a CH3 region comprising a knob modification comprises, or consists of:
an amino acid sequence having the amino acid sequence of SEQ ID NO:17, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:17; or
an amino acid sequence having the amino acid sequence of SEQ ID NO:18, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:18; or
an amino acid sequence having the amino acid sequence of SEQ ID NO:19, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:19; or
an amino acid sequence having the amino acid sequence of SEQ ID NO:20, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:20; or
an amino acid sequence having the amino acid sequence of SEQ ID NO:21, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:21;
wherein the amino acid sequence comprises 366W and 354C.

In some embodiments, a CH3 region comprising a hole modification comprises, or consists of:
an amino acid sequence having the amino acid sequence of SEQ ID NO:27, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:27; or
an amino acid sequence having the amino acid sequence of SEQ ID NO:28, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:28; or
an amino acid sequence having the amino acid sequence of SEQ ID NO:29, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:29; or
an amino acid sequence having the amino acid sequence of SEQ ID NO:30, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:30; or
an amino acid sequence having the amino acid sequence of SEQ ID NO:31, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:31;
wherein the amino acid sequence comprises 366S, 368A, 407V and 349C.

In some embodiments a polypeptide according to the present disclosure comprising a CH3 region comprises a CH2 region. The CH2 region is preferably adjacent to and immediately upstream of (i.e. N-terminal to) the CH3 region in the amino acid sequence of the polypeptide.

In some embodiments, a CH3 region according to the present disclosure is comprised within a CH2-CH3 region. That is, in some embodiments, a polypeptide according to the present disclosure comprising a CH3 region comprises a CH2-CH3 region.

In some embodiments, a CH2 region comprises, or consists of, an amino acid sequence having the amino acid sequence of SEQ ID NO:4, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:4. In some embodiments, a CH2 region comprises, or consists of, an amino acid sequence having the amino acid sequence of SEQ ID NO:32, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:32. In some embodiments, a CH2 region comprises, or consists of, an amino acid sequence having the amino acid sequence of SEQ ID NO:33, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:33. In some embodiments, a CH2 region comprises, or consists of, an amino acid sequence having the amino acid sequence of SEQ ID NO:34, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:34.

In some embodiments, a CH2-CH3 region comprises, or consists of, an amino acid sequence having the amino acid sequence of SEQ ID NO:35, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:35. In some embodiments, a CH2-CH3 region comprises, or consists of, an amino acid sequence having the amino acid sequence of SEQ ID NO:36, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:36.

It will be appreciated that the present disclosure provides CH2-CH3 regions comprising a CH3 region comprising a knob modification. Such CH2-CH3 regions may simply be described as CH2-CH3 regions comprising a knob modification.

Similarly, the present disclosure provides CH2-CH3 regions comprising a CH3 region comprising a hole modification. Such CH2-CH3 regions may simply be described as CH2-CH3 regions comprising a hole modification.

In some embodiments, a CH2-CH3 region comprising a knob modification comprises, or consists of:
an amino acid sequence having the amino acid sequence of SEQ ID NO:37, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:37; or
an amino acid sequence having the amino acid sequence of SEQ ID NO:38, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:38; or
an amino acid sequence having the amino acid sequence of SEQ ID NO:39, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:39; or
an amino acid sequence having the amino acid sequence of SEQ ID NO:40, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:40; or
an amino acid sequence having the amino acid sequence of SEQ ID NO:41, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:41; or
an amino acid sequence having the amino acid sequence of SEQ ID NO:42, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:42; or
an amino acid sequence having the amino acid sequence of SEQ ID NO:43, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:43; or
an amino acid sequence having the amino acid sequence of SEQ ID NO:44, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:44; or
an amino acid sequence having the amino acid sequence of SEQ ID NO:45, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:45; or
an amino acid sequence having the amino acid sequence of SEQ ID NO:46, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:46;
wherein the amino acid sequence comprises 366W.

In some embodiments, a CH2-CH3 region comprising a hole modification comprises, or consists of:
an amino acid sequence having the amino acid sequence of SEQ ID NO:47, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:47; or
an amino acid sequence having the amino acid sequence of SEQ ID NO:48, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:48; or
an amino acid sequence having the amino acid sequence of SEQ ID NO:49, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:49; or
an amino acid sequence having the amino acid sequence of SEQ ID NO:50, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:50; or
an amino acid sequence having the amino acid sequence of SEQ ID NO:51, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:51; or
an amino acid sequence having the amino acid sequence of SEQ ID NO:52, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:52; or
an amino acid sequence having the amino acid sequence of SEQ ID NO:53, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:53; or
an amino acid sequence having the amino acid sequence of SEQ ID NO:54, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:54; or
an amino acid sequence having the amino acid sequence of SEQ ID NO:55, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:55; or
an amino acid sequence having the amino acid sequence of SEQ ID NO:56, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:56;
wherein the amino acid sequence comprises 366S, 368A and 407V.

In some embodiments, a CH2-CH3 region comprising a knob modification comprises, or consists of:
an amino acid sequence having the amino acid sequence of SEQ ID NO:42, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:42; or
an amino acid sequence having the amino acid sequence of SEQ ID NO:43, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:43; or
an amino acid sequence having the amino acid sequence of SEQ ID NO:44, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:44; or
an amino acid sequence having the amino acid sequence of SEQ ID NO:45, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:45; or
an amino acid sequence having the amino acid sequence of SEQ ID NO:46, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:46;
wherein the amino acid sequence comprises 366W and 354C.

In some embodiments, a CH2-CH3 region comprising a hole modification comprises, or consists of:
an amino acid sequence having the amino acid sequence of SEQ ID NO:52, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:52; or
an amino acid sequence having the amino acid sequence of SEQ ID NO:53, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:53; or
an amino acid sequence having the amino acid sequence of SEQ ID NO:54, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:54; or
an amino acid sequence having the amino acid sequence of SEQ ID NO:55, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:55; or
an amino acid sequence having the amino acid sequence of SEQ ID NO:56, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:56;
wherein the amino acid sequence comprises 366S, 368A, 407V and 349C.

In some embodiments, a KiH molecule according to the present disclosure comprises an Fc moiety. As used herein, an ‘Fc moiety’ refers to a polypeptide complex formed by interaction between two polypeptides, wherein each polypeptide comprises a CH2-CH3 region.

In some embodiments, an Fc moiety according to the present disclosure comprises (i) a polypeptide comprising a CH2-CH3 region comprising a knob modification; and (ii) a polypeptide comprising a CH2-CH3 region comprising a hole modification.

In some embodiments, a KiH molecule or an Fc moiety according to the present disclosure comprises:
(a) (i) a polypeptide comprising an amino acid sequence having the amino acid sequence of SEQ ID NO:37, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:37, wherein the amino acid sequence comprises 366W; and (ii) a polypeptide comprising an amino acid sequence having the amino acid sequence of SEQ ID NO:47, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:47, wherein the amino acid sequence comprises 366S, 368A and 407V; or
(b) (i) a polypeptide comprising an amino acid sequence having the amino acid sequence of SEQ ID NO:38, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:38, wherein the amino acid sequence comprises 366W; and (ii) a polypeptide comprising an amino acid sequence having the amino acid sequence of SEQ ID NO:48, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:48, wherein the amino acid sequence comprises 366S, 368A and 407V; or
(c) (i) a polypeptide comprising an amino acid sequence having the amino acid sequence of SEQ ID NO:39, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:39, wherein the amino acid sequence comprises 366W; and (ii) a polypeptide comprising an amino acid sequence having the amino acid sequence of SEQ ID NO:49, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:49, wherein the amino acid sequence comprises 366S, 368A and 407V; or
(d) (i) a polypeptide comprising an amino acid sequence having the amino acid sequence of SEQ ID NO:40, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:40, wherein the amino acid sequence comprises 366W; and (ii) a polypeptide comprising an amino acid sequence having the amino acid sequence of SEQ ID NO:50, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:50, wherein the amino acid sequence comprises 366S, 368A and 407V; or
(e) (i) a polypeptide comprising an amino acid sequence having the amino acid sequence of SEQ ID NO:41, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:41, wherein the amino acid sequence comprises 366W; and (ii) a polypeptide comprising an amino acid sequence having the amino acid sequence of SEQ ID NO:51, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:51, wherein the amino acid sequence comprises 366S, 368A and 407V; or
(f) (i) a polypeptide comprising an amino acid sequence having the amino acid sequence of SEQ ID NO:42, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:42, wherein the amino acid sequence comprises 366W; and (ii) a polypeptide comprising an amino acid sequence having the amino acid sequence of SEQ ID NO:52, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:52, wherein the amino acid sequence comprises 366S, 368A and 407V; or
(g) (i) a polypeptide comprising an amino acid sequence having the amino acid sequence of SEQ ID NO:43, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:43, wherein the amino acid sequence comprises 366W; and (ii) a polypeptide comprising an amino acid sequence having the amino acid sequence of SEQ ID NO:53, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:53, wherein the amino acid sequence comprises 366S, 368A and 407V; or
(h) (i) a polypeptide comprising an amino acid sequence having the amino acid sequence of SEQ ID NO:44, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:44, wherein the amino acid sequence comprises 366W; and (ii) a polypeptide comprising an amino acid sequence having the amino acid sequence of SEQ ID NO:54, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:54, wherein the amino acid sequence comprises 366S, 368A and 407V; or
(i) (i) a polypeptide comprising an amino acid sequence having the amino acid sequence of SEQ ID NO:45, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:45, wherein the amino acid sequence comprises 366W; and (ii) a polypeptide comprising an amino acid sequence having the amino acid sequence of SEQ ID NO:55, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:55, wherein the amino acid sequence comprises 366S, 368A and 407V; or
(j) (i) a polypeptide comprising an amino acid sequence having the amino acid sequence of SEQ ID NO:46, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:46, wherein the amino acid sequence comprises 366W; and (ii) a polypeptide comprising an amino acid sequence having the amino acid sequence of SEQ ID NO:56, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or 99% sequence identity to SEQ ID NO:56, wherein the amino acid sequence comprises 366S, 368A and 407V.

In some embodiments, a KiH molecule or an Fc moiety according to the present disclosure comprises:
(a) (i) a polypeptide comprising an amino acid sequence having the amino acid sequence of SEQ ID NO:42, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:42, wherein the amino acid sequence comprises 366W and 354C; and (ii) a polypeptide comprising an amino acid sequence having the amino acid sequence of SEQ ID NO:52, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:52, wherein the amino acid sequence comprises 366S, 368A, 407V and 349C; or
(b) (i) a polypeptide comprising an amino acid sequence having the amino acid sequence of SEQ ID NO:43, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:43, wherein the amino acid sequence comprises 366W and 354C; and (ii) a polypeptide comprising an amino acid sequence having the amino acid sequence of SEQ ID NO:53, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:53, wherein the amino acid sequence comprises 366S, 368A, 407V and 349C; or
(c) (i) a polypeptide comprising an amino acid sequence having the amino acid sequence of SEQ ID NO:44, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:44, wherein the amino acid sequence comprises 366W and 354C; and (ii) a polypeptide comprising an amino acid sequence having the amino acid sequence of SEQ ID NO:54, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:54, wherein the amino acid sequence comprises 366S, 368A, 407V and 349C; or
(d) (i) a polypeptide comprising an amino acid sequence having the amino acid sequence of SEQ ID NO:45, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:45, wherein the amino acid sequence comprises 366W and 354C; and (ii) a polypeptide comprising an amino acid sequence having the amino acid sequence of SEQ ID NO:55, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:55, wherein the amino acid sequence comprises 366S, 368A, 407V and 349C; or
(e) (i) a polypeptide comprising an amino acid sequence having the amino acid sequence of SEQ ID NO:46, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:46, wherein the amino acid sequence comprises 366W and 354C; and (ii) a polypeptide comprising an amino acid sequence having the amino acid sequence of SEQ ID NO:56, or an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO:56, wherein the amino acid sequence comprises 366S, 368A, 407V and 349C.

In some embodiments, KiH molecules according to the present disclosure further comprise a target-binding moiety. In some embodiments, KiH molecules according to the present disclosure are target-binding molecules.

A ‘target-binding molecule’ refers to a molecule that binds to a given target molecule. Target-binding molecules include antigen-binding molecules such as antibodies (i.e. immunoglobulins (Igs)) and antigen-binding fragments thereof.

As used herein, ‘antibodies’ include monoclonal antibodies, polyclonal antibodies, monospecific and multispecific (e.g., bispecific, trispecific, etc.) antibodies, and antibody-derived antigen-binding molecules such as scFv, scFab, diabodies, triabodies, scFv-Fc, minibodies, single domain antibodies (e.g. VhH, etc.). Antigen-binding fragments of antibodies include e.g. Fv, Fab, F(ab′)₂ and F(ab′) fragments. Antigen-binding molecules also include antibody-derived molecules, e.g. molecules comprising an antigen-binding region/domain derived from an antibody. Antibody-derived antigen-binding molecules may comprise an antigen-binding region/domain that comprises, or consists of, the antigen-binding region of an antibody (e.g. an antigen-binding fragment of an antibody). In some embodiments, the antigen-binding region/domain of an antibody-derived antigen-binding molecule may be or comprise the Fv (e.g. provided as an scFv) or the Fab region of an antibody, or the whole antibody.

The target-binding molecules of the present disclosure comprise one or more target-binding moieties. Target-binding moieties are moieties capable of binding to a target molecule. Binding of a target-binding moiety to a target-binding molecule may be characterised by non-covalent interaction, electrostatic interaction (e.g. ionic bonding, hydrogen bonding) and/or Van der Waals forces.

Target-binding moieties contemplated in connection with the present disclosure include antigen-binding moieties, and peptides/polypeptides/complexes derived from interaction partners for target molecules.

In some embodiments, the target-binding molecules of the present disclosure comprise one or more antigen-binding moieties. Antigen-binding moieties are moieties capable of binding to a given target antigen.

Antigen-binding moieties may be derived from antibodies. Antibody-derived antigen-binding moieties may comprise, or consist of, the antigen-binding region of an antibody (e.g. an antigen-binding fragment of an antibody). In some embodiments, an antigen-binding moiety may be or comprise the Fv (e.g. provided as an scFv) or the Fab region of an antibody that binds to a given target antigen, or the whole antibody.

In some embodiments, an antigen-binding moiety comprises an antibody heavy chain variable region (VH) and an antibody light chain variable region (VL) of an antibody capable of specific binding to the target antigen. In some embodiments, an antigen-binding moiety comprises or consists of an aptamer capable of binding to the target antigen, e.g. a nucleic acid aptamer (reviewed, for example, in Zhou and Rossi Nat Rev Drug Discov. (2017) 16(3):181-202).

An antigen-binding moiety may be formed of an antigen-binding peptide/polypeptide, or an antigen-binding peptide/polypeptide complex. An antigen-binding moiety may comprise more than one peptide/polypeptide which together form an antigen-binding moiety. The peptides/polypeptides may associate to form the antigen-binding moiety by protein:protein interaction. By way of example, in certain embodiments described herein, KiH molecules of the present disclosure comprise an antigen-binding moiety (particularly a Fab moiety) formed by association between: (i) a polypeptide comprising a VH region and CH1 region (and further comprising a CH2-CH3 region according to the present disclosure), and (ii) a polypeptide comprising a VL region and CL region.

In some embodiments, antigen-binding moiety comprises or consists of an antigen-binding peptide/polypeptide or an antigen-binding peptide/polypeptide complex. An antigen-binding polypeptide may be e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr Top Med Chem. (2015) 15(12):1082-1101, which is hereby incorporated by reference in its entirety (see also e.g. Boersma et al., J Biol Chem. (2011) 286:41273-85 and Emanuel et al., Mabs (2011) 3:38-48).

In some embodiments, an antigen-binding moiety according to the present disclosure comprises, or consists of, a polypeptide complex formed by protein:protein interaction between constituent peptides/polypeptides of the antigen-binding moiety.

By way of illustration, in certain embodiments described herein, KiH molecules of the present disclosure comprise an antigen-binding moiety consisting of an antigen-binding polypeptide comprising (i) a polypeptide comprising the VH region of an antibody that binds to FAP, and (ii) a polypeptide comprising the VL region of the antibody that binds to FAP. In some embodiments, KiH molecules of the present disclosure comprise an antigen-binding moiety consisting of an antigen-binding polypeptide comprising (i) a polypeptide comprising the VH region of an antibody that binds to BCMA, and (ii) a polypeptide comprising the VL region of the antibody that binds to BCMA. In some embodiments, KiH molecules of the present disclosure comprise an antigen-binding moiety consisting of an antigen-binding polypeptide comprising (i) a polypeptide comprising the VH region of an antibody that binds to CD19, and (ii) a polypeptide comprising the VL region of the antibody that binds to CD19. In some embodiments, KiH molecules of the present disclosure comprise an antigen-binding moiety consisting of an antigen-binding polypeptide comprising (i) a polypeptide comprising the VH region of an antibody that binds to CEA, and (ii) a polypeptide comprising the VL region of the antibody that binds to CEA. In some embodiments, KiH molecules of the present disclosure comprise an antigen-binding moiety consisting of an antigen-binding polypeptide comprising (i) a polypeptide comprising the VH region of an antibody that binds to HER2, and (ii) a polypeptide comprising the VL region of the antibody that binds to HER2. In some embodiments, KiH molecules of the present disclosure comprise an antigen-binding moiety consisting of an antigen-binding polypeptide comprising (i) a polypeptide comprising the VH region of an antibody that binds to PD-L1, and (ii) a polypeptide comprising the VL region of the antibody that binds to PD-L1.

It will be appreciated that in some embodiments, a constituent polypeptide of a KiH molecule according to the present disclosure comprises an amino acid sequence forming all or part of an antigen-binding moiety.

In some embodiments, a constituent polypeptide of a KiH molecule according to the present disclosure comprises an antigen-binding moiety. In some embodiments, a constituent polypeptide of a KiH molecule according to the present disclosure comprises an antigen-binding peptide/polypeptide.

In some embodiments, a constituent polypeptide of a KiH molecule according to the present disclosure comprises part of an antigen-binding moiety. In some embodiments, a constituent polypeptide of a KiH molecule according to the present disclosure comprises a component/fragment of an antigen-binding moiety. By way of illustration, in certain embodiments described herein, KiH molecules comprise a polypeptide comprising the VH of an antigen-binding moiety specific for a target antigen, and such molecules further comprise a polypeptide comprising the VL of the antigen-binding moiety.

In some embodiments, a target-binding moiety according to the present disclosure may comprise, or consist of, an antigen peptide/polypeptide or peptide/polypeptide complex which is, or which is derived from, an interaction partner for the target molecule to which it binds. For example, an target-binding moiety may comprise, or may consist of, a peptide/polypeptide with an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to the amino acid sequence of the target-interacting region of an interaction partner for the target molecule to which it binds.

An interaction partner for a target molecule may be a peptide/polypeptide or peptide/polypeptide complex with which the target molecule interacts, e.g. via protein:protein interaction. In some embodiments, an interaction partner for a target molecule may be a peptide/polypeptide with which the target molecule interacts to form a peptide/polypeptide complex (i.e. a peptide/polypeptide complex comprising the target molecule and the interaction partner for the target molecule).

A ‘target-interacting region’ of an interaction partner for a target molecule refers to the region(s)/domain(s) of the molecule through which the interaction partner interacts with the target molecule, e.g. through protein:protein interaction. The region may comprise the amino acids that contact the target molecule. The target-interacting region of an interaction partner for a target molecule may also be referred to as the ‘target-binding region’. By way of illustration, the ‘target-interacting region’ of 4-1BBL is the extracellular domain of 4-1BBL, which is formed by positions 24 to 186 of UniProtKB: Q07011-1, v1.

In some embodiments, the interaction partner for a target molecule and the target molecule interact with one another in the manner of a receptor:ligand interaction. In some embodiments, a target-binding moiety comprises, or consists of, a peptide/polypeptide with an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to the amino acid sequence of the target-interacting region of a ligand or receptor for the antigen to which it binds. By way of illustration, in certain embodiments described herein, KiH molecules of the present disclosure comprise a target-binding peptide/polypeptide or a target-binding peptide/polypeptide complex comprising an amino acid sequence corresponding to the amino acid sequence of the extracellular domain of 4-1BBL (which is the ligand for 4-1BB) or a fragment thereof.

In some embodiments, a target-binding moiety according to the present disclosure comprises, or consists of, a target-binding peptide/polypeptide or a target-binding peptide/polypeptide complex comprising a plurality of (e.g. two, three or more) non-overlapping amino acid sequences, each having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to the amino acid sequence of the target-interacting region of an interaction partner for the target molecule to which it binds (e.g. target-binding region of a ligand or receptor for the molecule to which it binds).

In some embodiments, the plurality of non-overlapping amino acid sequences are provided in tandem in the amino acid sequence of the target-binding peptide/polypeptide, or a peptide/polypeptide of the target-binding peptide/polypeptide complex. In some embodiments, the plurality of non-overlapping amino acid sequences are connected to one another via linker sequence(s).

Linker sequences are known to the skilled person, and are described, for example in Chen et al., Adv Drug Deliv Rev. (2013) 65(10):1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev. (2013) 65(10):1357-1369. Flexible linker sequences often comprise high proportions of glycine and/or serine residues. In some embodiments, a linker sequence comprises at least one glycine residue and/or at least one serine residue. In some embodiments, the linker sequence comprises or consists of glycine and serine residues. In some embodiments, the linker sequence has the structure: (GₓS)n or (GₓS)ₙGₘ; wherein G=glycine, S=serine, x=3 or 4, n=2, 3, 4, 5 or 6, and m=0, 1, 2 or 3.

In some embodiments, the linker sequence comprises one or more (e.g. 1, 2, 3, 4, 5 or 6) copies (e.g. in tandem) of the sequence motif G₄S (SEQ ID NO:66). In some embodiments, a linker sequence comprises or consists of (G₄S)₂ (SEQ ID NO:67), (G₄S)₃ (SEQ ID NO:68) or (G₄S)₄ (SEQ ID NO:69). In some embodiments, the linker sequence has a length of 1-2, 1-3, 1-4, 1-5, 1-10, 1-15, 1-20, 1-25, or 1-30 amino acids.

In some embodiments, a target-binding moiety according to the present disclosure comprises, or consists of, a ligand for a costimulatory molecule. Accordingly, in some embodiments, KiH molecules according to the present disclosure comprise a ligand for a costimulatory molecule. Costimulatory molecules include e.g. 4-1BB, CD28, OX40, ICOS and CD27, and ligands for costimulatory molecules include e.g. 4-1BBL, CD86, CD80, OX40L ICOSL and CD70). A ‘ligand for a costimulatory molecule’ as referred to herein, is any entity to which a costimulatory molecule binds.

In some embodiments, a target-binding moiety according to the present disclosure comprises or consists of a ligand for 4-1BB, comprising an amino acid sequence corresponding to the amino acid sequence of the extracellular domain of 4-1BBL, or a fragment thereof. The extracellular domain of 4-1BBL is formed by positions 24 to 186 of UniProtKB: Q07011-1, v1.

4-1BBL (or ‘4-1BB ligand’ or ‘CD137L’) is a costimulatory TNF ligand family member, which is able to costimulate proliferation and cytokine production of T cells. Costimulatory TNF family ligands can costimulate TCR signals upon interaction with their corresponding TNF receptors and the interaction with their receptors leads to recruitment of TNFR-associated factors (TRAF), which initiate signalling cascades that result in T cell activation. 4-1BBL is a type II transmembrane protein. Complete or full-length 4-1BBL having the amino acid sequence shown in UniProt accession no. P41273 (entry version 153) has been described to form trimers on the surface of cells. The formation of trimers is enabled by specific motifs of the ectodomain of 4-1BBL. Said motifs are designated herein as ‘trimerization region’. The amino acids 50-254 of the human 4-1BBL sequence (SEQ ID NO:57) form the extracellular domain of 4-1BBL, but even fragments thereof are able to form the trimers.

An ‘extracellular domain’ is the domain of a membrane protein that extends into the extracellular space (i.e. the space outside the cell), also referred to as ‘ectodomain’. The extracellular domain of 4-1BBL as defined herein refers to the part of the 4-1BBL protein, particularly the human 4-1BBL protein (UniProt accession no. P41273 (entry version 153)) that extends into the extracellular space, but also includes shorter parts or fragments thereof that are responsible for the trimerization and for the binding to the corresponding receptor 4-1BB. In specific aspects of the present disclosure, the extracellular domain of 4-1BBL is formed by positions 24 to 186 of UniProtKB: Q07011-1, v1.

The term ‘extracellular domain of 4-1BBL or a fragment thereof’ thus refers to the extracellular domain of 4-1BBL, or to parts thereof that are able to bind to 4-1BB and are capable of trimerization. In specific aspects of the present disclosure, the term ‘extracellular domain of 4-1BBL or a fragment thereof’ refers to a polypeptide having an amino acid sequence selected from SEQ ID NO:57 (amino acids 50-254 of human 4-1BBL), SEQ ID NO:58 (amino acids 71-254 of human 4-1BBL), SEQ ID NO:59 (amino acids 85-254 of human 4-1BBL), SEQ ID NO:60 (amino acids 80-254 of human 4-1BBL), SEQ ID NO:61 (amino acids 52-254 of human 4-1BBL), SEQ ID NO:62 (amino acids 71-248 of human 4-1BBL), SEQ ID NO:63 (amino acids 85-248 of human 4-1BBL), SEQ ID NO:64 (amino acids 80-248 of human 4-1BBL) and SEQ ID NO:65 (amino acids 52-248 of human 4-1BBL).

In some embodiments, a target-binding moiety according to the present disclosure comprises, or consists of, a polypeptide having an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity an amino acid sequence selected from SEQ ID NO:57, SEQ ID NO:58, SEQ ID NO:59, SEQ ID NO:60, SEQ ID NO:61, SEQ ID NO:62, SEQ ID NO:63, SEQ ID NO:64 and SEQ ID NO:65.

In some embodiments, a target-binding moiety according to the present disclosure comprises, or consists of, a polypeptide having plurality of (e.g. two, three or more) non-overlapping amino acid sequences, each having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to the amino acid sequence of the target-interacting region of an interaction partner for a target molecule. By way of illustration, in certain embodiments described herein, KiH molecules of the present disclosure comprise a 4-1BB-binding moiety comprising three amino acid sequences, each corresponding to the amino acid sequence of the extracellular domain of 4-1BBL or a fragment thereof.

In some embodiments, a target-binding moiety according to the present disclosure comprises, or consists of, a polypeptide having plurality of (e.g. two, three or more) non-overlapping amino acid sequences, each having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to an amino acid sequence selected from SEQ ID NO:57, SEQ ID NO:58, SEQ ID NO:59, SEQ ID NO:60, SEQ ID NO:61, SEQ ID NO:62, SEQ ID NO:63, SEQ ID NO:64 and SEQ ID NO:65.

In some embodiments, the plurality of non-overlapping amino acid sequences are provided in tandem in the amino acid sequence of the polypeptide of the target-binding moiety. In some embodiments, the plurality of non-overlapping amino acid sequences are connected to one another via linker sequence(s). By way of illustration, in certain embodiments described herein, KiH molecules of the present disclosure comprise a 4-1BB-binding moiety comprising a polypeptide comprising two amino acid sequences corresponding to the amino acid sequence of the extracellular domain of 4-1BBL or a fragment thereof, connected by linker sequences.

In some embodiments, a target-binding moiety according to the present disclosure comprises, or consists of, a polypeptide comprising an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to an amino acid sequence selected from SEQ ID NO:70, SEQ ID NO:71, SEQ ID NO:72, SEQ ID NO:73, SEQ ID NO:74, SEQ ID NO:75, SEQ ID NO:76, and SEQ ID NO:77.

In some embodiments, a target-binding moiety according to the present disclosure comprises, or consists of, a polypeptide complex formed by protein:protein interaction between constituent peptides/polypeptides of the target-binding moiety. By way of illustration, in certain embodiments described herein, KiH molecules of the present disclosure comprise a target-binding moiety consisting of (i) a polypeptide comprising two amino acid sequences corresponding to the amino acid sequence of the extracellular domain of 4-1BBL or a fragment thereof, connected by linker sequences, and (ii) a polypeptide comprising the extracellular domain of 4-1BBL or a fragment thereof. In such embodiments, polypeptides (i) and (ii) associate to form the complete target-binding moiety.

It will be appreciated that in some embodiments, a constituent polypeptide of a KiH molecule according to the present disclosure comprises an amino acid sequence forming all or part of a target-binding moiety.

In some embodiments, a constituent polypeptide of a KiH molecule according to the present disclosure comprises a target-binding moiety. In some embodiments, a constituent polypeptide of a KiH molecule according to the present disclosure comprises a target-binding peptide/polypeptide. By way of illustration, in certain embodiments described herein, KiH molecules comprise a polypeptide comprising a ligand for a 4-1BB comprising an amino acid sequence corresponding to the amino acid sequence of the extracellular domain of 4-1BBL.

In some embodiments, a constituent polypeptide of a KiH molecule according to the present disclosure comprises part of a target-binding moiety. In some embodiments, a constituent polypeptide of a KiH molecule according to the present disclosure comprises a component/fragment of target-binding moiety.

In some embodiments, the constituent polypeptides of a polypeptide complex forming a target-binding moiety according to the present disclosure comprise one or more regions facilitating/potentiating protein:protein interaction between the polypeptides. For example, in some embodiments, one of the polypeptides may comprise a CH1 region, and the other polypeptide may comprise a CL region.

By way of illustration, in certain embodiments described herein, KiH molecules of the present disclosure comprise an antigen-binding moiety consisting of an antigen-binding polypeptide comprising (i) a polypeptide comprising the VH region of an antibody that binds to FAP and a CH1 region, and (ii) a polypeptide comprising the VL region of the antibody that binds to FAP and a CL region. In some embodiments, KiH molecules of the present disclosure comprise an antigen-binding moiety consisting of an antigen-binding polypeptide comprising (i) a polypeptide comprising the VH region of an antibody that binds to BCMA and a CH1 region, and (ii) a polypeptide comprising the VL region of the antibody that binds to BCMA and a CL region. In some embodiments, KiH molecules of the present disclosure comprise an antigen-binding moiety consisting of an antigen-binding polypeptide comprising (i) a polypeptide comprising the VH region of an antibody that binds to CD19 and a CH1 region, and (ii) a polypeptide comprising the VL region of the antibody that binds to CD19 and a CL region. In some embodiments, KiH molecules of the present disclosure comprise an antigen-binding moiety consisting of an antigen-binding polypeptide comprising (i) a polypeptide comprising the VH region of an antibody that binds to CEA and a CH1 region, and (ii) a polypeptide comprising the VL region of the antibody that binds to CEA and a CL region. In some embodiments, KiH molecules of the present disclosure comprise an antigen-binding moiety consisting of an antigen-binding polypeptide comprising (i) a polypeptide comprising the VH region of an antibody that binds to HER2 and a CH1 region, and (ii) a polypeptide comprising the VL region of the antibody that binds to HER2 and a CL region. In some embodiments, KiH molecules of the present disclosure comprise an antigen-binding moiety consisting of an antigen-binding polypeptide comprising (i) a polypeptide comprising the VH region of an antibody that binds to PD-L1 and a CH1 region, and (ii) a polypeptide comprising the VL region of the antibody that binds to PD-L1 and a CL region.

By way of further illustration, in certain embodiments described herein, KiH molecules of the present disclosure comprise a 4-1BB-binding moiety consisting of (i) a polypeptide comprising two amino acid sequences corresponding to the amino acid sequence of the extracellular domain of 4-1BBL or a fragment thereof, connected by linker sequences, followed by a CL region, and (ii) a polypeptide comprising the extracellular domain of 4-1BBL or a fragment thereof, followed by a CH1 region.

In some embodiments, the CL region and/or CH1 region may comprise modification to promote their association. In some embodiments, the CL region comprises modification to promote association with a CH1 region. In some embodiments, the CH1 region comprises modification to promote association with a CL region. In some embodiments, the CH1 region and the CL region comprise paired modifications promoting their association. Such modifications include modifications promoting electrostatic interaction between the CH1 region and the CL region. For example, the modifications may introduce amino acid residues that participate in the formation of salt bridges between polypeptides comprising the CH1 and CL regions.

Paired modifications promoting association of CH1 and CL regions are described e.g. in WO 2015/150447 A1, which is hereby incorporated by reference in its entirety. Such modifications include the CH1 region modifications K147E and K213E, and the CL region modifications E123R and Q124K (EU numbering). In this example, a salt bridge is formed between the E147 COO— group and the K124 NH₃⁺ group, and another is formed between the E213 COO— group and the R123 NH₃⁺ group, thereby stabilising interaction between the CH1 and CL regions.

In some embodiments, a CH1 region comprising modification to promote association with a CL region has an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:81 and comprises K147E and K213E. In some embodiments, a CH1 region has an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:82 and comprises E147 and E213.

In some embodiments, a CL region comprising modification to promote association with a CH1 region has an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:82 and comprises E123R and Q124K. In some embodiments, a CL region has an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:82 and comprises R123 and K124.

In some embodiments, a target-binding moiety according to the present disclosure comprises: (i) a polypeptide comprising an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:81, and comprising E147 and E213, and (ii) a polypeptide comprising an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:82, and comprising R123 and K124.

In some embodiments, a target-binding moiety according to the present disclosure comprises, or consists of: (i) a polypeptide having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:78; and (ii) a polypeptide having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:80.

In some embodiments, a KiH molecule according to the present disclosure comprises: (i) a polypeptide having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:78; and (ii) a polypeptide having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% sequence identity to SEQ ID NO:79.

The target molecule for a target-binding moiety according to the present disclosure may be any target molecule. In some embodiments, a target molecule may be a peptide/polypeptide, glycoprotein, lipoprotein, glycan, glycolipid, lipid, or fragment thereof. A target molecule may be expressed at the cell surface of a cell expressing the target molecule.

In some embodiments, a target molecule is an antigen. As used herein, an ‘antigen’ refers to a molecule capable of eliciting an immune response in a subject.

In some embodiments, a target molecule is a disease-associated antigen. A ‘disease-associated antigen’ refers to an antigen whose presence is indicative of a given disease/disease state, or an antigen for which an elevated level of the antigen is positively-correlated with a given disease/disease state. The disease-associated antigen may be an antigen whose expression is associated with the development, progression or severity of symptoms of a given disease. The disease-associated antigen may be associated with the cause or pathology of the disease, or may be expressed abnormally as a consequence of the disease. A disease-associated antigen may be an antigen of an infectious agent or pathogen, a cancer-associated antigen or an autoimmune disease-associated antigen.

In some embodiments, the disease-associated antigen is an antigen of a pathogen. The pathogen may be prokaryotic (bacteria), eukaryotic (e.g. protozoan, helminth, fungus), virus or prion. In some embodiments, the pathogen is an intracellular pathogen. In some embodiments the pathogen is a virus, e.g. a virus as described hereinabove. In some embodiments the pathogen is a bacterium.

In some embodiments, the target molecule is a cancer-associated antigen. A cancer-associated antigen is an antigen whose expression or overexpression is associated with cancer. In some embodiments, the cancer-associated antigen is a receptor molecule, e.g. a cell surface receptor. In some embodiments, the cancer-associated antigen is a cell signalling molecule, e.g. a cytokine, chemokine, interferon, interleukin or lymphokine. In some embodiments, the cancer-associated antigen is a growth factor or a hormone. In some embodiments, the cancer-associated antigen is a viral antigen. A cancer cell antigen may be abnormally expressed by a cancer cell (e.g. the cancer cell antigen may be expressed with abnormal localisation), or may be expressed with an abnormal structure by a cancer cell. A cancer cell antigen may be capable of eliciting an immune response. In some embodiments, the antigen is expressed at the cell surface of the cancer cell (i.e. the cancer cell antigen is a cancer cell surface antigen). In some embodiments, the part of the antigen which is bound by an antigen-binding molecule described herein is displayed on the external surface of the cancer cell (i.e. is extracellular). The cancer cell antigen may be a cancer-associated antigen. In some embodiments the cancer cell antigen is an antigen whose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e.g. at the RNA and/or protein level) by cells of a cancer, e.g. as compared to the level of expression by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e.g. non-cancerous cells derived from the same tissue/cell type). In some embodiments, the cancer-associated antigen may be the product of a mutated oncogene or mutated tumor suppressor gene. In some embodiments, the cancer-associated antigen may be the product of an overexpressed cellular protein, a cancer antigen produced by an oncogenic virus, an oncofetal antigen, or a cell surface glycolipid or glycoprotein.

Cancer-associated antigens are reviewed by Zarour H M, DeLeo A, Finn O J, et al. Categories of Tumor Antigens. In: Kufe D W, Pollock R E, Weichselbaum R R, et al., editors. Holland-Frei Cancer Medicine. 6^{th} edition. Hamilton (ON): B C Decker; 2003. Cancer-associated antigens include oncofetal antigens: CEA, Immature laminin receptor, TAG-72; oncoviral antigens such as HPV E6 and E7; overexpressed proteins: fibroblast activation protein (FAP), B-cell maturation antigen (BCMA), CD19, HER2/neu, BING-4, calcium-activated chloride channel 2, cyclin-B1, 9D7, Ep-CAM, EphA3, telomerase, mesothelin, SAP-1, survivin; cancer-testis antigens: BAGE, CAGE, GAGE, MAGE, SAGE, XAGE, CT9, CT10, NY-ESO-1, PRAME, SSX-2; lineage restricted antigens: MART1, Gp100, tyrosinase, TRP-½2, MC1R, prostate specific antigen; mutated antigens: β-catenin, BRCAM½, CDK4, CML66, Fibronectin, MART-2, p53, Ras, TGF-βRII; post-translationally altered antigens: MUC1, idiotypic antigens: Ig, TCR. Other cancer cell antigens include heat-shock protein 70 (HSP70), heat-shock protein 90 (HSP90), glucose-regulated protein 78 (GRP78), vimentin, nucleolin, feto-acinar pancreatic protein (FAPP), alkaline phosphatase placental-like 2 (ALPPL-2), siglec-5, stress-induced phosphoprotein 1 (STIP1), protein tyrosine kinase 7 (PTK7), and cyclophilin B. In some embodiments the cancer cell antigen is a cancer cell antigen described in Zhao and Cao, Front Immunol. (2019) 10:2250, which is hereby incorporated by reference in its entirety.

In some embodiments, the target molecule is selected from FAP, BCMA, CD19, CEA, HER2 and PD-L1. In some embodiments, the target molecule is FAP. In some embodiments, the target molecule is BCMA. In some embodiments, the target molecule is CD19. In some embodiments, the target molecule is CEA. In some embodiments, the target molecule is HER2. In some embodiments, the target molecule is PD-L1.

In some embodiments, the target molecule is an immune cell surface molecule. An immune cell surface molecule is any molecule which is expressed in or at the cell membrane of an immune cell. In some embodiments, the part of the immune cell surface molecule which is bound by the antigen-binding moiety is on the external surface of the immune cell (i.e. is extracellular). The immune cell surface molecule may be expressed at the cell surface of any immune cell. In some embodiments, the immune cell may be a cell of hematopoietic origin, e.g. a neutrophil, eosinophil, basophil, dendritic cell, lymphocyte, or monocyte. The lymphocyte may be e.g. a T cell, B cell, natural killer (NK) cell, NKT cell or innate lymphoid cell (ILC), or a precursor thereof (e.g. a thymocyte or pre-B cell). The immune cell may express a CD3 polypeptide (e.g. CD3γ CD3ε CD3ζ or CD3δ), a TCR polypeptide (TCRα or TCRβ), CD27, CD28, CD4 or CD8. In some embodiments, the immune cell is a T cell, e.g. a CD3+ T cell. In some embodiments, the T cell is a CD3+, CD4+ T cell. In some embodiments, the T cell is a CD3+, CD8+ T cell. In some embodiments, the T cell is a T helper cell (TH cell). In some embodiments, the T cell is a cytotoxic T cell (e.g. a cytotoxic T lymphocyte (CTL)). In some embodiments, the immune cell is a T cell or an NK cell.

In some embodiments, an immune cell surface molecule may be a CD3-TCR complex polypeptide, e.g. TCRα, TCRβ, TCRγ, TCRδ, TRAC, TRBC1, TRBC2, TRGC1, TRGC2, TRDC, CD3ε, CD3δ, CD3γ, CD3ζ or CD3η. In some embodiments, an immune cell surface molecule is CD3, CD8, CD4 or CD28. In some embodiments, an immune cell surface molecule is a checkpoint molecule (e.g. PD-1, CTLA-4, LAG-3, TIM-3, VISTA, TIGIT or BTLA), or a ligand for a checkpoint molecule (e.g. PD-L1, PD-L2, CD80, CD86, MHC class I, MHC Class II, Galectin 9, VSIG3, VSIG8, LRIG1, PSGL1, CD155 or HVEM). In some embodiments the immune cell surface molecule is a costimulatory molecule (e.g. CD28, OX40, 4-1BB, ICOS or CD27), or a ligand for a costimulatory molecule (e.g. CD86, CD80, OX40L 4-1BBL, ICOSL or CD70).

It will be appreciated that the KiH molecules of the present disclosure are preferably multispecific. By ‘multispecific’ it is meant that the molecules bind to more than one target molecule. The multispecific KiH molecules of the present disclosure comprise at least two, non-identical target-binding moieties.

By way of illustration, the multispecific KiH molecules described herein comprise a FAP-binding moiety (specifically, a FAP-binding Fab moiety), and a 4-1BB-binding moiety (specifically, a 4-1BBL-derived, 4-1BB-binding moiety). In some embodiments, a multispecific KiH molecule comprises a FAP-binding moiety (e.g. a FAP-binding Fab moiety), and a 4-1BB-binding moiety (e.g. a 4-1BBL-derived, 4-1BB-binding moiety). In some embodiments, a multispecific KiH molecule comprises a BCMA-binding moiety (e.g. a BCMA-binding Fab moiety), and a 4-1BB-binding moiety (e.g. a 4-1BBL-derived, 4-1BB-binding moiety). In some embodiments, a multispecific KiH molecule comprises a CD19-binding moiety (e.g. a CD19-binding Fab moiety), and a 4-1BB-binding moiety (e.g. a 4-1BBL-derived, 4-1BB-binding moiety). In some embodiments, a multispecific KiH molecule comprises a CEA-binding moiety (e.g. a CEA-binding Fab moiety), and a 4-1BB-binding moiety (e.g. a 4-1BBL-derived, 4-1BB-binding moiety). In some embodiments, a multispecific KiH molecule comprises a HER2-binding moiety (e.g. a HER2-binding Fab moiety), and a 4-1BB-binding moiety (e.g. a 4-1BBL-derived, 4-1BB-binding moiety). In some embodiments, a multispecific KiH molecule comprises a HER2-binding moiety (e.g. a PD-L1-binding Fab moiety), and a PD-L1-binding moiety (e.g. a 4-1BBL-derived, 4-1BB-binding moiety).

It will be appreciated that multispecific KiH molecules are at least bispecific. The term ‘bispecific’ means that the KiH molecule binds to at least two, distinct targets. In some embodiments, the KiH molecule is bispecific, trispecific, tetraspecific, pentaspecific, hexaspecific, heptaspecific, octaspecific, nonaspecific or decaspecific.

The multispecific KiH molecules according to the present disclosure display at least monovalent binding with respect to a first target molecule, and also display at least monovalent binding with respect to a second target molecule. Binding valency refers to the number of binding sites in a KiH molecule for a given target molecule.

In some aspects and embodiments, a KiH molecule according to the present disclosure may comprise a polypeptide complex formed by protein:protein interaction between polypeptides having a structures (a) and (b) according to one of (1) to (7) below:
(1)
(a) N-term-[ . . . ]-[CH3 region comprising a knob modification]-[ . . . ]-C-term
(b) N-term-[ . . . ]-[CH3 region comprising a hole modification]-[ . . . ]-C-term
(2)
(a) N-term-[ . . . ]-[CH2 region]-[CH3 region comprising a knob modification]-[ . . . ]-C-term
(b) N-term-[ . . . ]-[CH2 region]-[CH3 region comprising a hole modification]-[ . . . ]-C-term
(3)
(a) N-term-[ . . . ]-[target-binding moiety/fragment of a target-binding moiety]-[ . . . ]-[CH3 region comprising a knob modification]-[ . . . ]-C-term
(b) N-term-[ . . . ]-[target-binding moiety/fragment of a target-binding moiety]-[ . . . ]-[CH3 region comprising a hole modification]-[ . . . ]-C-term
(4)
(a) N-term-[ . . . ]-[target-binding moiety/fragment of a target-binding moiety]-[ . . . ]-[CH3 region comprising a knob modification]-[ . . . ]-C-term
(b) N-term-[ . . . ]-[target-binding moiety/fragment of a target-binding moiety]-[ . . . ]-[CH3 region comprising a hole modification]-[ . . . ]—C-term
(5)
(a) N-term-[ . . . ]-[antigen-binding peptide/polypeptide]-[ . . . ]—[CH3 region comprising a hole modification]-[ . . . ]—C-term
(b) N-term-[ . . . ]-[antigen-binding moiety/fragment of an antigen-binding moiety]-[ . . . ]—[CH3 region comprising a knob modification]-[ . . . ]—C-term
(6)
(a) N-term-[ . . . ]-[target-binding peptide/polypeptide comprising an amino acid sequence corresponding to the amino acid sequence of the target-interacting region of an interaction partner for a target molecule]-[ . . . ]—[CH3 region comprising a knob modification]-[ . . . ]—C-term (b) N-term-[VH/VL region of an antibody that binds to a target antigen]-[ . . . ]—[CH3 region comprising a hole modification]-[ . . . ]—C-term
(7)
(a) N-term-[ . . . ]-[target-binding peptide/polypeptide comprising an amino acid sequence corresponding to the amino acid sequence of the target-interacting region of an interaction partner for a target molecule]-[ . . . ]—[CH3 region comprising a hole modification]-[ . . . ]—C-term
(b) N-term-[ . . . ]-[VH/VL region of an antibody that binds to a target antigen]-[ . . . ]—[CH3 region comprising a knob modification]-[ . . . ]—C-term

As used in representations of polypeptide structures herein, ‘[ . . . ]’ indicates the optional presence of further amino acid sequence(s)/protein domain(s). For example, in the structure of (1)(a) above, further sequences of amino acids/protein domain(s) may optionally be present upstream of (i.e. N-terminal to) the amino acid sequence of the CH3 region, before the N terminus of the polypeptide. Furthermore, as used in representations of polypeptide structures herein ‘-’ indicates an optional linker sequence (e.g. a linker sequence as described hereinabove). For example, protein domains may be connected to one another via linker sequences.

It will be appreciated that the KiH molecule according to the present disclosure may comprise further polypeptides in addition to those conforming to (a) and (b) in (1) to (7) above. For example, in connection with (7) above, the KiH molecule may further comprise a polypeptide providing the VH or VL region of the relevant antibody that is not provided in the polypeptide of (b), in order to form the complete antigen-binding Fv moiety.

### Cell Comprising Nucleic Acid Encoding Polypeptides According to the Present Disclosure

The methods for producing KiH molecules comprise culturing cells comprising nucleic acid encoding the constituent polypeptides of the KiH molecules.

The present disclosure provides methods comprising culturing cells comprising a nucleic acid, or a plurality of nucleic acids, encoding polypeptides according to the present disclosure (e.g. the constituent polypeptides of a KiH molecule described herein). In some embodiments, the nucleic acid(s) comprise or consist of DNA and/or RNA.

The polypeptides of the present disclosure may be produced within a cell by translation of RNA encoding the polypeptides. The polypeptides of the present disclosure may be produced within a cell by transcription from nucleic acid encoding the polypeptides, and subsequent translation of the transcribed RNA.

In some embodiments, the nucleic acid(s) may be, or may be comprised/contained in, a vector, or a plurality of vectors. A ‘vector’ as used herein is a nucleic acid molecule used as a vehicle to transfer exogenous nucleic acid into a cell. Thus a cell according to the present disclosure may comprise a vector, or plurality of vectors, comprising the nucleic acid or plurality of nucleic acids according to the present disclosure. The constituent polypeptides of the molecules of the present disclosure may be encoded by nucleic acid provided in non-identical vectors. For example, one or more of the constituent polypeptides of a KiH molecule according to the present disclosure may be encoded by nucleic acid of a first vector, and one or more of the constituent polypeptides of the KiH molecule may be encoded by nucleic acid of a second vector. In some embodiments, a vector may comprise multiple, non-overlapping copies of nucleic acid encoding a constituent polypeptide of a KiH molecule according to the present disclosure.

A vector may facilitate delivery of nucleic acid(s) encoding the polypeptides according to the present disclosure to a cell. A vector may be an expression vector, comprising elements required for expressing the polypeptides according to the present disclosure. A vector may comprise elements facilitating integration of nucleic acid(s) into the genomic DNA of cell into which the vector is introduced.

A vector may be a vector for expression of the nucleic acid in the cell (i.e. an expression vector). Such vectors may include a promoter sequence operably linked to a nucleotide sequence encoding a polypeptide according to the present disclosure. A vector may also include a termination codon (i.e. 3′ in the nucleotide sequence of the vector to the nucleotide sequence encoding a polypeptide) and expression enhancers. Any suitable vectors, promoters, enhancers and termination codons known in the art may be used to express a peptide or polypeptide from a vector according to the present disclosure.

The term ‘operably linked’ may include the situation where nucleic acid encoding a polypeptide according to the present disclosure and regulatory nucleic acid sequence(s) (e.g. a promoter and/or enhancers) are covalently linked in such a way as to place the expression of the nucleic acid encoding a polypeptide under the influence or control of the regulatory nucleic acid sequence(s) (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into the desired polypeptide(s).

Vectors contemplated in connection with the present disclosure include DNA vectors, RNA vectors, plasmids (e.g. conjugative plasmids (e.g. F plasmids), non-conjugative plasmids, R plasmids, col plasmids, episomes), viral vectors (e.g. retroviral vectors, e.g. gammaretroviral vectors (e.g. murine Leukemia virus (MLV)-derived vectors, e.g. SFG vector), lentiviral vectors, adenovirus vectors, adeno-associated virus vectors, vaccinia virus vectors and herpesvirus vectors), transposon-based vectors, and artificial chromosomes (e.g. yeast artificial chromosomes), e.g. as described in Maus et al., Annu Rev Immunol. (2014) 32:189-225 and Morgan and Boyerinas, Biomedicines (2016) 4:9, which are both hereby incorporated by reference in their entirety. In some embodiments, a vector according to the present disclosure is a lentiviral vector.

In some embodiments, the vector may be a eukaryotic vector, i.e. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

It will be appreciated that where cells are referred to herein in the singular (i.e. ‘a/the cell’), pluralities/populations of such cells are also contemplated.

A cell according to the present disclosure may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In preferred embodiments, the cell is a human cell.

In some embodiments, the cell is, or is derived from, a cell type commonly used for the expression of polypeptides for use in therapy in humans. Exemplary cells are described e.g. in Kunert and Reinhart, Appl Microbiol Biotechnol. (2016) 100:3451-3461 (hereby incorporated by reference in its entirety), and include e.g. CHO, HEK 293, PER.C6, NS0 and BHK cells. In preferred embodiments, the cell is, or is derived from, a CHO cell.

Any suitable method may be employed to produce a cell to be employed in the methods of the present disclosure. Such methods may comprise nucleic acid transfer for permanent (i.e. stable) or transient expression of the transferred nucleic acid. In some embodiments, following introduction into a cell, nucleic acid(s) encoding the polypeptide(s) of interest may be integrated into or form part of the genomic DNA of the cell. In some embodiments, following introduction into a cell, nucleic acid(s) encoding the polypeptide(s) of interest may be maintained extrachromosomally.

Any suitable genetic engineering platform may be used, and include gammaretroviral vectors, lentiviral vectors, adenovirus vectors, DNA transfection, transposon-based gene delivery and RNA transfection, for example as described in Maus et al., Annu Rev Immunol. (2014) 32:189-225, hereby incorporated by reference in its entirety. Methods also include those described e.g. in Wang and Riviere Mol Ther Oncolytics. (2016) 3:16015, which is hereby incorporated by reference in its entirety. Suitable methods for introducing nucleic acid(s)/vector(s) into cells include transduction, transfection and electroporation.

### Methods for Producing KiH Molecules

Aspects and embodiments of the present disclosure relate to methods for producing KiH molecules.

The methods comprise culturing cells comprising nucleic acid encoding the constituent polypeptides of the KiH molecule under conditions suitable for expression of the polypeptides and assembly of the KiH molecule.

Suitable culture conditions will be apparent to a person skilled in the art. Culture conditions for the expression of antibodies from mammalian cells in culture are described e.g. in Birch and Racher, Adv Drug Deliv Rev. (2006) 58(5-6):671-85 and Li et al., MAbs (2010) 2(5):466-477, both of which are hereby incorporated by reference in their entirety. Suitable culture conditions include conditions suitable for the maintenance of cells of the CHO-K1 cell line (ATCC, Cat. No. CCL-61) in in vitro culture.

The cells are cultured in cell culture medium comprising amino acids, vitamins, inorganic salts and sugars. In some embodiments, the cell culture medium comprises amino acids selected from: L-alanine, L-arginine, L-asparagine, L-aspartic acid, L-cystine/L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. In some embodiments, the cell culture medium comprises vitamins and/or vitaminoids selected from: D-biotin, choline chloride, D-calcium pantothenate, folic acid, myo-inositol, niacinamide, pyridoxine hydrochloride, riboflavin, thiamine hydrochloride, and vitamin B12. In some embodiments, the cell culture medium comprises further components selected from: calcium chloride, hypoxanthine, ferric nitrate, linoleic acid, putrescine hydrochloride, pyruvic acid, magnesium sulfate, potassium chloride, sodium bicarbonate, sodium chloride, sodium phosphate monobasic, thioctic acid and thymidine. In some embodiments, the cell culture medium comprises D-glucose.

In some embodiments, the cell culture medium is a cell culture medium suitable for the culture of mammalian cells. Such cell culture media include Roswell Park Memorial Institute (RPMI) 1640 medium, Dulbecco's Modified Eagle Medium (DMEM), F-12 medium, DMEM/F12, CD-CHO medium and PowerCHO medium.

In preferred embodiments, the cell culture medium is suitable cell culture medium for the culture of cells for the production of molecules to be employed in therapy in humans. Such culture medium includes e.g. EX-CELL Advanced CHO Fed-Batch Medium.

It will be appreciated that the cells are cultured under suitable environmental conditions.

The cells may be cultured at 35.5° C. to 37.5° C., e.g. at one of about 35.5° C., about 36° C., about 36.5° C., about 37° C., or about 37.5° C.

The cells may be cultured in 4% to 10% CO₂, e.g. 5% to 8% CO₂.

The cells may be cultured e.g. at ≥90% humidity, e.g. about 95% humidity.

The cells may be cultured without agitation, or with agitation. Agitation may be at 75 rpm to 175 rpm, e.g. 90 rpm to 130 rpm, e.g. about 110 rpm.

The pH of the cell culture may be between 6.8 to 7.4, e.g. one of about 6.8, 6.9, 7.0, 7.1, 7.2, 7.3 or 7.4. In some embodiments, the pH of the cell culture is about 7.0. In some embodiments, the pH of the cell culture is about 7.2.

Cell culture may be performed in a bioreactor provided with an appropriate supply of nutrients, air/oxygen and/or growth factors. Bioreactors may monitor and control environmental conditions such as pH, oxygen, flow rates into and out of, and agitation within the vessel such that optimum conditions are provided for the cells being cultured.

The culture may be a continuous culture, with a continuous flow of cell culture medium into, and a continuous flow of cultured cells from, the cell culture vessel. In some embodiments, the culture may be a batch culture, employing a closed system and a finite amount of cell culture medium. In some embodiments, the culture may be a fed-batch culture, in which cell culture medium is supplied to the cell culture vessel during the culture, but wherein unlike a continuous culture, material is not removed from the cell culture vessel during the course of cell culture.

In some embodiments, the methods comprise adding one or more components to the cell culture during the period of culture. In some embodiments, the methods comprise adding cell culture medium to the cell culture during the period of culture. In some embodiments, the methods comprise adding nutrients to the cell culture during the period of culture. In some embodiments, the methods comprise adding amino acids to the cell culture during the period of culture.

In some embodiments, components added to the cell culture during the period of culture as described in the preceding paragraph do not comprise cysteine (i.e. do not comprise species having the formula: HOOC—CH(—NH₂)—CH₂—SH, ⁻OOC—CH(—N⁺H₃)—CH₂—SH, ⁻OOC—CH(—NH₂)—CH₂—SH (or a salt thereof), or HOOC—CH(—N⁺H₃)—CH₂—SH (or a salt thereof)). That is, in some embodiments, cell culture medium, nutrient and/or amino acids added to the cell culture during the period of culture do not comprise cysteine (i.e. are cysteine-free).

Following a period of cell culture, KiH molecules may be separated/isolated/purified, e.g. from the cell culture or cell culture medium.

The KiH molecules are typically secreted into the cell culture medium from the cells in culture. Secreted KiH molecules can be collected by partitioning culture medium from the cells (e.g. by centrifugation), and subsequently isolating/purifying the secreted KiH molecules from the culture medium.

Techniques for the isolation/purification of antigen-binding molecules from compositions comprising heterogeneous populations of proteins are well known to those of skill in the art, and are described, for example, in Green and Sambrook, Molecular Cloning: A Laboratory Manual (4th Edition), Birch and Racher, Adv Drug Deliv Rev. (2006) 58(5-6):671-85, and Murphy et al., Antibody Technology Journal (2016) 6:17-32, all of which are hereby incorporated by reference in their entirety.

Large-scale purification of antigen-binding molecules is commonly based on affinity chromatography, and Protein A or G affinity purification is used in many cases. Purification may alternatively, or additionally, comprise purification by anion/cation exchange chromatography, hydrophobic interaction chromatography and/or size exclusion chromatography, which are well known to the person skilled in the art.

The various purification steps are designed to remove contaminant proteins from the cells or culture media to ppm levels, and to reduce DNA to ppb levels. Depending on the processes used, there may be additional specific contaminants to be removed (e.g. leached protein A/G). Purification may comprise filtration (e.g. using a 0.22 μm filter) to remove potential biological contaminants.

In addition to contaminants, it may also be desirable or necessary to remove undesired products such as hole-hole molecules, knob-knob molecules, ½ hole molecules and ½ knob molecules, and other undesired products such as aggregates and degradation products.

As used herein, a ‘hole-hole molecule’ is a molecule comprising a polypeptide complex formed by interaction between a first polypeptide comprising a CH3 region comprising a hole modification and a second polypeptide comprising a CH3 region comprising a hole modification. In some embodiments, the first and second polypeptides are the same, such that the ‘hole-hole molecule’ is a homomeric polypeptide complex (e.g. a homodimer). Similarly, a ‘knob-knob molecule’ is a molecule comprising a polypeptide complex formed by interaction between a first polypeptide comprising a CH3 region comprising a knob modification and a second polypeptide comprising a CH3 region comprising a knob modification. In some embodiments, the first and second polypeptides are the same, such that the ‘knob-knob molecule’ is a homomeric polypeptide complex (e.g. a homodimer). A ‘½ hole molecule’ refers to a polypeptide/polypeptide complex comprising a CH3 region comprising a hole modification that has not associated with another polypeptide comprising a CH3 region comprising a knob modification to form a KiH molecule, and which is not a hole-hole molecule. Similarly, a ‘½ knob molecule’ refers to a polypeptide/polypeptide complex comprising a CH3 region comprising a knob modification that has not associated with another polypeptide comprising a CH3 region comprising a hole modification to form a KiH molecule, and which is not a knob-knob molecule.

In connection with various aspects and embodiments of the present disclosure, an ‘undesired product’ may be selected from a hole-hole molecule, a knob-knob molecule, a ½ hole molecule, and a ½ knob molecule.

In some embodiments, isolating or purifying a KiH molecule according to the present disclosure comprises isolation/purification by one or more of: affinity chromatography, (e.g. Protein G chromatography or Protein A chromatography), size exclusion chromatography, high-performance liquid chromatography, ultra-performance liquid chromatography, and ion-exchange chromatography. In some embodiments, the methods of the present disclosure comprise separating/isolating/purifying polypeptide complexes by size-exclusion chromatography and/or capillary electrophoresis.

Following isolation/purification, the KiH molecule may be provided in a suitable buffer, e.g. for storage. As used herein, a ‘buffer’ refers to a buffered solution that resists changes in pH by the action of its acid-base conjugate components. A composition comprising a KiH molecule according to the present disclosure may comprise the KiH molecule in a buffer.

Examples of suitable buffers include acetate, histidine, histidine-arginine, histidine-methionine and other organic acid buffers. KiH molecules may be buffer exchanged into a buffer of interest by buffer dialysis.

In some embodiments, methods of the present disclosure comprise formulating a KiH molecule according to the present disclosure to a composition, e.g. a pharmaceutical composition. In some embodiments the methods comprise mixing a KiH molecule, or mixing a composition comprising a KiH molecule, with a pharmaceutically-acceptable carrier, diluent, excipient or adjuvant.

### Cysteine Concentrations

Aspects and embodiments of the present disclosure relate to methods for producing KiH molecules comprising culturing cells comprising nucleic acid encoding the constituent polypeptides of KiH molecules, in the presence of certain concentrations of thiol/selenol group-containing species.

The present disclosure is based in part on the inventors' discovery that culturing cells comprising nucleic acid encoding the constituent polypeptides of KiH molecules in cell culture medium comprising relatively low concentrations of thiol/selenol group-containing species (e.g. cysteine) favours the production of such molecules over undesired products (e.g. hole-hole molecules, ½ hole molecules and/or ½ knob molecules). As demonstrated in the experimental examples, maintaining relatively low levels of cysteine in the cell culture increases the proportion of KiH molecules among the molecules expressed from the cells.

Without wishing to be bound by any particular theory, the inventors consider that the increased formation of KiH molecules in conditions where cell culture medium comprises relatively low concentrations of thiol group-containing species (e.g. cysteine) may arise as a consequence of the increased availability of thiol groups of cysteine residues of the CH3 knob and CH3 hole regions to form intermolecular disulfide bonds.

Accordingly, the present disclosure provides the production of KiH molecules from cells comprising nucleic acid encoding the constituent polypeptides of KiH molecules in cell culture, wherein the concentration of thiol/selenol group-containing species (e.g. cysteine) is provided and/or maintained at low levels.

As used herein, a ‘thiol group’ refers to a group having the structure ‘R—SH’, and a ‘selenol group’ refers to a group having the structure ‘R—SeH’. In both cases, ‘R’ represents an alkyl or other organic substituent. Species comprising a thiol group include e.g. cysteine, N-acetyl cysteine, N-acetyl-L-cysteine amide (NACA), S-sulfocysteine, N,N′-diacetyl-l-cystine, N,N′-diacetyl-L-cystine dimethyl ester (DACDM), glutathione, dithiothreitol and 2-mercaptoethanol. Species comprising a selenol group include e.g. selenocysteine.

More particularly, aspects and embodiments of the present disclosure relate to methods for producing KiH molecules comprising culturing cells comprising nucleic acid encoding the constituent polypeptides of KiH molecules, in the presence of certain concentrations of cysteine.

Herein, ‘cysteine’ refers to the species having the structure of (1) or (2):

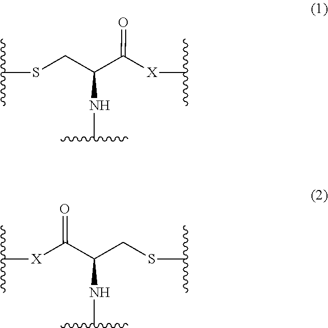

wherein ‘X’ is O or N.

The species having the structure of (1) or (2) are comprised e.g. in cysteine (i.e. HOOC—CH(—NH₂)—CH₂—SH), OOC—CH(—N⁺H₃)—CH₂—SH, ⁻OOC—CH(—NH₂)—CH₂—SH (and salts thereof), and HOOC—CH(—N⁺H₃)—CH₂—SH (and salts thereof), in their L and D enantiomeric forms. The species are also comprised in cystine (i.e. L-cystine and D-cystine), which is a dimer of cysteine formed by a disulfide bond between the thiol groups of two cysteine monomers. Cystine can be imported into cells via the xCT transporter, and subsequently reduced to cysteine by the action of cystine reductase.

The concentration of cysteine in solution (e.g. in cell culture medium) in accordance with the present disclosure may be calculated as the concentration of the species having the structure of (1) or (2) above. For example, calculations of the concentration of cysteine also take into account the concentration of cystine (e.g. L-cystine).

Accordingly, a given concentration of cystine equates to a concentration of cysteine that is double the concentration of cystine. By way of illustration, in accordance with the present disclosure, cell culture medium comprising 1 mM cystine (and lacking any other form of cysteine) is considered to have a concentration of cysteine of 2 mM.

Cell cultures according to the present disclosure may comprise both monomeric cysteine, and cystine. In such embodiments, the concentration of cysteine in the cell culture is determined by combining the concentration of monomeric cysteine and the concentration of cystine. By way of illustration, in accordance with the present disclosure, cell culture medium comprising cystine at a concentration of 1 mM and monomeric cysteine at a concentration of 2 mM is considered to have a cumulative concentration of cysteine molar equivalent of 4 mM.

In aspects and embodiments of the present disclosure, methods for producing KiH molecules comprise culturing cells comprising nucleic acid encoding the constituent polypeptides of KiH molecules in cell culture medium comprising cysteine at a concentration of less than 8 mM. In some embodiments, the concentration of cysteine in the cell culture medium is >0 mM and <8 mM. In some embodiments, the concentration of cysteine in the cell culture medium is ≥0.5 mM and <8 mM. In preferred embodiments, the concentration of cysteine in the cell culture medium is less than 6 mM. In some embodiments, the concentration of cysteine in the cell culture medium is >0 mM and <6 mM. In some embodiments, the concentration of cysteine in the cell culture medium is ≥1 mM and <6 mM. In some embodiments, the concentration of cysteine in the cell culture medium is ≥2 mM and ≤5 mM. In some embodiments, the concentration of cysteine in the cell culture medium is ≥2.5 mM and ≤4.75 mM. In some embodiments, the concentration of cysteine in the cell culture medium is about 3 mM. In some embodiments, the concentration of cysteine in the cell culture medium is about 4.5 mM.

In aspects and embodiments of the present disclosure, the methods comprise culturing cells comprising nucleic acid encoding the constituent polypeptides of KiH molecules in cell culture medium comprising cysteine at a concentration according to an embodiment of the preceding paragraph for the majority of the period of culture.

That is, in some embodiments the methods comprise culturing cells comprising nucleic acid encoding the constituent polypeptides of KiH molecules for the majority of the period of culture in cell culture medium comprising cysteine at a concentration of less than 8 mM. In some embodiments, the concentration of cysteine in the cell culture medium for the majority of the period of culture is >0 mM and <8 mM. In some embodiments, the concentration of cysteine in the cell culture medium for the majority of the period of culture is ≥0.5 mM and <8 mM. In some embodiments, the concentration of cysteine in the cell culture medium for the majority of the period of culture is less than 6 mM. In some embodiments, the concentration of cysteine in the cell culture medium for the majority of the period of culture is >0 mM and <6 mM. In some embodiments, the concentration of cysteine in the cell culture medium for the majority of the period of culture is ≥1 mM and <6 mM. In some embodiments, the concentration of cysteine in the cell culture medium for the majority of the period of culture is ≥2 mM and ≤5 mM. In some embodiments, the concentration of cysteine in the cell culture medium for the majority of the period of culture is ≥2.5 mM and 4.75 mM. In some embodiments, the concentration of cysteine in the cell culture medium for the majority of the period of culture is about 3 mM. In some embodiments, the concentration of cysteine in the cell culture medium for the majority of the period of culture is about 4.5 mM.

Herein, ‘the majority of the period of culture’ is >50% of the period of culture. By way of illustration, where the period of culture is 14 days, the majority of the period of culture is >7 days out of the 14 day period. In some embodiments, the concentration of cysteine in the culture is maintained at the relevant concentration for the majority of period of culture.

In some embodiments, the methods comprise culturing cells comprising nucleic acid encoding the constituent polypeptides of KiH molecules for ≥50%, e.g. one of ≥60%, ≥70%, ≥80%, ≥90% or ≥95% of the period of culture, or for the duration of the period of culture, in cell culture medium comprising cysteine at a concentration of less than 8 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is ≥0 mM and <8 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is ≥0.5 mM and <8 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is less than 6 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is ≥0 mM and <6 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is ≥1 mM and <6 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is ≥2 mM and ≤5 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is ≥2.5 mM and ≤4.75 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is about 3 mM. In some embodiments, the concentration of cysteine in the cell culture medium for ≥50%, e.g. one of ≥60%, ≥70%, ≥80%, ≥90% or ≥95% of the period of culture, or for the duration of the period of culture, is about 4.5 mM.

In some aspects and embodiments of the present disclosure, the cell culture may be established (i.e. ‘set up’) in cell culture medium comprising cysteine at a concentration of less than 8 mM. The methods may comprise ‘seeding’ the cells in cell culture medium comprising cysteine at a specified concentration. It will be appreciated that ‘establishing’ a cell culture or ‘seeding’ cells comprises contacting cells with cell culture medium, e.g. within an appropriate cell culture vessel.

Accordingly, in some aspects and embodiments of the present disclosure, the methods comprise seeding cells comprising nucleic acid encoding the constituent polypeptides of KiH molecules in cell culture medium comprising cysteine at a concentration of less than 8 mM. In some embodiments, the cells are seeded in cell culture medium comprising cysteine at a concentration of ≥0.5 mM and <8 mM. In preferred embodiments, the cells are seeded in cell culture medium comprising cysteine at a concentration of less than 6 mM. In some embodiments, the cells are seeded in cell culture medium comprising cysteine at a concentration of ≥1 mM and <6 mM. In some embodiments, the cells are seeded in cell culture medium comprising cysteine at a concentration of ≥2 mM and ≤5 mM. In some embodiments, the cells are seeded in cell culture medium comprising cysteine at a concentration of ≥2.5 mM and ≤4.75 mM. In some embodiments, the cells are seeded in cell culture medium comprising cysteine at a concentration of about 3 mM. In some embodiments, the cells are seeded in cell culture medium comprising cysteine at a concentration of about 4.5 mM.

In some aspects and embodiments according to the present disclosure, the methods comprise maintaining the concentration of cysteine within the cell culture at a concentration of less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM) for the majority of the period of culture (e.g. one of ≥60%, ≥70%, ≥80%, ≥90% or ≥95% of the period of culture, or for the duration of the period of culture).

The concentration of cysteine in the cell culture naturally decreases over time (i.e. where further cysteine is not added to the culture) as a consequence of metabolism of the cysteine by the cells in culture and/or its degradation.

The concentration of cysteine in a given cell culture may be maintained below a certain threshold (e.g. <8 mM, <6 mM, <5 mM, <4.75 mM) either by not adding cysteine to the cell culture during the period of culture, or by only introducing cysteine to the cell culture during the period of culture at a concentration not exceeding the relevant threshold. For example, in embodiments wherein the culture is a fed-batch or continuous culture, cell culture medium added to the cell culture during the period of culture may lack cysteine (i.e. may be cysteine-free), or may comprise cysteine at a concentration not exceeding the relevant threshold, or may comprise cysteine at a concentration such that following addition of the cell culture medium to the culture, the concentration of cysteine in the cell culture does not exceed the relevant threshold (i.e. is <8 mM, <6 mM, <5 mM or <4.75 mM).

Conversely, the concentration of cysteine in a given cell culture may be maintained at or above a certain threshold value (e.g. ≥0.5 mM, ≥1 mM, ≥2 mM), or at a certain value (e.g. about 3 mM or 4.5 mM), by introducing cysteine to the cell culture during the period of culture at a concentration such that following its addition to the cell culture, the concentration of cysteine in the cell culture is at or above the relevant threshold value, or at the relevant certain value. For example, in embodiments wherein the culture is a fed-batch or continuous culture, cell culture medium added to the cell culture during the period of culture may comprise cysteine at a concentration such that following addition of the cell culture medium to the culture, the concentration of cysteine in the cell culture is one of ≥0.5 mM, ≥1 mM, ≥2 mM, or about 3 mM, or 4.5 mM.

Where cysteine is provided to a cell culture in accordance with the present disclosure, it may be provided in the form of cell culture medium, or a cell culture medium supplement or additive, comprising monomeric cysteine, cystine, or a metabolic precursor or derivative of cysteine/cystine capable of metabolism to cysteine by the cells in culture. A metabolic precursor or derivative of cysteine/cystine according to the present disclosure may be a molecule comprising the structure of (1) or (2):

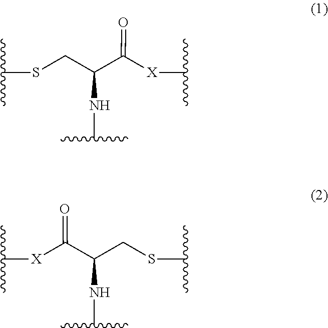

wherein ‘X’ is O or N.

Metabolic precursors and derivatives of cysteine/cystine contemplated in accordance with the present disclosure include N-acetyl-cysteine (NAC), N-acetyl-L-cysteine amide (NACA), S-sulfocysteine, N,N′-diacetyl-l-cystine, and N,N′-diacetyl-L-cystine dimethyl ester (DACDM).

### Osmolality

Aspects and embodiments of the present disclosure relate to methods for producing KiH molecules comprising culturing cells comprising nucleic acid encoding the constituent polypeptides of KiH molecules, at certain osmolality.

Osmolality refers to the number of solute particles per kilogram of solvent. Osmolality is expressed as the number of osmoles (e.g. milliosmoles (mOsmol)) of solute per kilogram of solvent (e.g. mOsmol/kg). Osmolality is distinct from osmolarity, which instead refers to the number of solute particles per litre of solvent.

The present disclosure is based in part on the inventors' discovery that culturing cells comprising nucleic acid encoding the constituent polypeptides of KiH molecules in cell culture medium at certain osmolality increases the yield of such molecules. As demonstrated in the experimental examples, maintaining relatively high osmolality increases the production of KiH molecules.

Accordingly, in aspects and embodiments of the present disclosure, methods for producing KiH molecules comprise culturing cells comprising nucleic acid encoding the constituent polypeptides of KiH molecules in cell culture medium having an osmolality of greater than 280 mOsmol/kg, e.g. >300 mOsmol/kg, e.g. ≥320 mOsmol/kg, e.g. ≥350 mOsmol/kg, e.g. ≥370 mOsmol/kg.

In accordance with the various aspects and embodiments described herein wherein a lower threshold osmolality value is specified, it will be appreciated that the osmolality is not so high as to significantly impair the growth of cells in cell culture and/or expression of KiH molecules according to the present disclosure from such cells. For example, in some embodiments, the osmolality of the cell culture medium of a cell culture according to the present disclosure is <600 mOsmol/kg, e.g. <550 mOsmol/kg, e.g. <500 mOsmol/kg, e.g. <475 mOsmol/kg.

In some embodiments, the osmolality of the cell culture medium is >280 mOsmol/kg and <450 mOsmol/kg. In preferred embodiments, the osmolality of the cell culture medium is >300 mOsmol/kg. In some embodiments, the osmolality of cell culture medium is >300 mOsmol/kg and <420 mOsmol/kg. In some embodiments, the osmolality of the cell culture medium is ≥320 mOsmol/kg and ≤400 mOsmol/kg. In some embodiments, the osmolality of the cell culture medium is ≥350 mOsmol/kg and 390 mOsmol/kg. In some embodiments, the osmolality of the cell culture medium is about 370 mOsmol/kg.

In aspects and embodiments of the present disclosure, the methods comprise culturing cells comprising nucleic acid encoding the constituent polypeptides of KiH molecules in cell culture medium having an osmolality according to an embodiment of the preceding paragraph for the majority of the period of culture.

That is, in some embodiments the methods comprise culturing cells comprising nucleic acid encoding the constituent polypeptides of KiH molecules for the majority of the period of culture in cell culture medium having an osmolality of greater than 280 mOsmol/kg. In some embodiments, the osmolality of the cell culture medium for the majority of the period of culture is greater than 300 mOsmol/kg. In some embodiments, the osmolality of the cell culture medium for the majority of the period of culture is ≥320 mOsmol/kg. In some embodiments, the osmolality of the cell culture medium for the majority of the period of culture is ≥350 mOsmol/kg. In some embodiments, the osmolality of the cell culture medium for the majority of the period of culture is ≥370 mOsmol/kg.

In some embodiments, the methods comprise culturing cells comprising nucleic acid encoding the constituent polypeptides of KiH molecules for ≥50%, e.g. one of ≥60%, ≥70%, ≥80%, ≥90% or ≥95% of the period of culture, or for the duration of the period of culture, in cell culture medium having an osmolality of greater than 280 mOsmol/kg. In some embodiments, the osmolality of the cell culture medium for ≥50%, e.g. one of ≥60%, ≥70%, ≥80%, ≥90% or ≥95% of the period of culture, or for the duration of the period of culture, is greater than 300 mOsmol/kg. In some embodiments, the osmolality of the cell culture medium for >50%, e.g. one of ≥60%, ≥70%, ≥80%, ≥90% or ≥95% of the period of culture, or for the duration of the period of culture, is ≥320 mOsmol/kg. In some embodiments, the osmolality of the cell culture medium for >50%, e.g. one of ≥60%, ≥70%, ≥80%, ≥90% or ≥95% of the period of culture, or for the duration of the period of culture, is ≥350 mOsmol/kg. In some embodiments, the osmolality of the cell culture medium for >50%, e.g. one of ≥60%, ≥70%, ≥80%, ≥90% or ≥95% of the period of culture, or for the duration of the period of culture, is ≥370 mOsmol/kg.

In some aspects and embodiments of the present disclosure, the cell culture may be established (i.e. ‘set up’) in cell culture medium having an osmolality of greater than 280 mOsmol/kg. The methods may comprise ‘seeding’ the cells in cell culture medium having a specified osmolality.

Accordingly, in some aspects and embodiments of the present disclosure, the methods comprise seeding cells comprising nucleic acid encoding the constituent polypeptides of KiH molecules in cell culture medium having an osmolality of greater than 280 mOsmol/kg. In some embodiments, the cells are seeded in cell culture medium having an osmolality>280 mOsmol/kg and ≤450 mOsmol/kg. In preferred embodiments, the cells are seeded in cell culture medium having an osmolality greater than 300 mOsmol/kg. In some embodiments, the cells are seeded in cell culture medium having an osmolality>300 mOsmol/kg and <420 mOsmol/kg. In some embodiments, the cells are seeded in cell culture medium having an osmolality≥320 mOsmol/kg and ≤400 mOsmol/kg. In some embodiments, the cells are seeded in a culture medium having an osmolality of ≥320 mOsmol/kg. In some embodiments, the cells are seeded in cell culture medium having an osmolality≥350 mOsmol/kg and 390 mOsmol/kg. In some embodiments, the cells are seeded in cell culture medium having an osmolality of about 370 mOsmol/kg.

In some aspects and embodiments according to the present disclosure, the methods comprise maintaining the osmolality of the cell culture at greater than 280 mOsmol/kg (e.g. >280 mOsmol/kg, e.g. >300 mOsmol/kg, e.g. ≥320 mOsmol/kg, e.g. ≥350 mOsmol/kg, e.g. ≥370 mOsmol/kg) for the majority of the period of culture (e.g. one of ≥60%, ≥70%, ≥80%, ≥90% or ≥95% of the period of culture, or for the duration of the period of culture).

The osmolality of the cell culture may naturally change over time (i.e. in the absence of intervention to manipulate the osmolality of the cell culture) as a consequence of addition of basic species (often in the form of sodium salts) for pH control, metabolism of salts in the cell culture medium and/or the secretion of osmolality-modifying products (e.g. waste products) from the cells.

The osmolality of a given cell culture may be maintained at or above a certain threshold value (e.g. >280 mOsmol/kg, >300 mOsmol/kg, ≥320 mOsmol/kg, ≥350 mOsmol/kg, ≥370 mOsmol/kg), by introducing an osmolality-increasing agent to the cell culture during the period of culture, in an amount such that following its addition to the culture, the osmolality of the cell culture medium is at or above the relevant threshold value, or at the relevant certain value. For example, in embodiments wherein the culture is a fed-batch or continuous culture, cell culture medium added to the cell culture during the period of culture may comprise an osmolality-increasing agent in an amount such that following addition of the cell culture medium to the culture, the concentration of osmolality of the cell culture medium in the cell culture is one of >280 mOsmol/kg, >300 mOsmol/kg, ≥320 mOsmol/kg, ≥350 mOsmol/kg or ≥370 mOsmol/kg.

An osmolality-increasing agent may be any agent that contributes to the osmotic pressure of a solution. Osmolality-increasing agents include organic compounds (e.g. carbohydrates, e.g. glucose, threalose, galactose, etc.) and inorganic compounds, e.g. inorganic salts. In some embodiments, an osmolality-increasing agent is a salt, e.g. an inorganic salt. In some embodiments, the salt may be selected from: sodium chloride, potassium chloride, sodium bicarbonate, calcium chloride, ferric nitrate, magnesium sulfate and sodium phosphate monobasic. In preferred embodiments, the osmolality-increasing agent is sodium chloride. Where an osmolality-increasing agent is provided to a cell culture in accordance with the present disclosure, it may be provided in the form of cell culture medium comprising the relevant agent, or a cell culture medium supplement or additive comprising the relevant agent.

Conversely, the osmolality of a given cell culture may be maintained at or below a certain threshold value (e.g. <600 mOsmol/kg, e.g. <550 mOsmol/kg, e.g. <500 mOsmol/kg, e.g. <475 mOsmol/kg), by diluting the cell culture medium of the cell culture during the period of culture (e.g. with cell culture medium having a lower osmolality than the osmolality of the cell culture medium to which it is added, or with buffer, or water), such that following dilution, the osmolality of the cell culture medium in the cell culture is at or above the relevant threshold value, or at the relevant certain value. For example, in embodiments wherein the culture is a fed-batch or continuous culture, cell culture medium added to the cell culture during the period of culture may have an osmolality such that following addition of the cell culture medium to the culture, the osmolality of the cell culture medium of the cell culture is <600 mOsmol/kg, e.g. <550 mOsmol/kg, e.g. <500 mOsmol/kg, e.g. <475 mOsmol/kg.

### Further Methods and Uses

The present disclosure provides further methods relating to the production of KiH molecules.

A method for increasing the yield of KiH molecules produced from cells comprising nucleic acid encoding the constituent polypeptides of the KiH molecule in cell culture is provided, wherein the method comprises maintaining or adjusting the concentration of cysteine in the cell culture medium of the cell culture to less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM). In some embodiments, the method further comprises maintaining or adjusting the osmolality of the cell culture medium of the cell culture to greater than 280 mOsmol/kg (e.g. >280 mOsmol/kg, e.g. >300 mOsmol/kg, e.g. ≥320 mOsmol/kg, e.g. ≥350 mOsmol/kg, e.g. ≥370 mOsmol/kg).

Also provided is a method for increasing the proportion of KiH molecules among molecules produced from cells comprising nucleic acid encoding the constituent polypeptides of the KiH molecule in cell culture, wherein the method comprises maintaining or adjusting the concentration of cysteine in the cell culture medium of the cell culture to less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM). In some embodiments, the method further comprises maintaining or adjusting the osmolality of the cell culture medium of the cell culture to greater than 280 mOsmol/kg (e.g. >280 mOsmol/kg, e.g. >300 mOsmol/kg, e.g. ≥320 mOsmol/kg, e.g. ≥350 mOsmol/kg, e.g. ≥370 mOsmol/kg).

Also provided is a method for reducing the yield of an undesired product (e.g. a hole-hole molecule, a ½ hole molecule, a ½ knob molecule, or a knob-knob molecule) produced from cells comprising nucleic acid encoding the constituent polypeptides of a KiH molecule in cell culture, wherein the method comprises maintaining or adjusting the concentration of cysteine in the cell culture medium of the cell culture to less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM). In some embodiments, the method further comprises maintaining or adjusting the osmolality of the cell culture medium of the cell culture to greater than 280 mOsmol/kg (e.g. >280 mOsmol/kg, e.g. >300 mOsmol/kg, e.g. ≥320 mOsmol/kg, e.g. ≥350 mOsmol/kg, e.g. ≥370 mOsmol/kg).

Also provided is a method for reducing the proportion of an undesired product (e.g. a hole-hole molecule, a ½ hole molecule, a ½ knob molecule or a knob-knob molecule) among molecules produced from cells comprising nucleic acid encoding the constituent polypeptides of a KiH molecule in cell culture, wherein the method comprises maintaining or adjusting the concentration of cysteine in the cell culture medium of the cell culture to less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM). In some embodiments, the method further comprises maintaining or adjusting the osmolality of the cell culture medium of the cell culture to greater than 280 mOsmol/kg (e.g. >280 mOsmol/kg, e.g. >300 mOsmol/kg, e.g. ≥320 mOsmol/kg, e.g. ≥350 mOsmol/kg, e.g. ≥370 mOsmol/kg).

In some embodiments, ‘maintaining or adjusting the concentration of cysteine in the cell culture medium of the cell culture’ may comprise increasing the concentration of cysteine in the cell culture. In such embodiments, the methods may comprise adding cysteine to the cell culture (e.g. in the form of monomeric cysteine, cystine, or a metabolic precursor or derivative of cysteine/cystine capable of metabolism to cysteine (e.g. as described hereinabove, e.g. N-acetyl-cysteine (NAC), N-acetyl-L-cysteine amide (NACA), S-sulfocysteine, N,N′-diacetyl-l-cystine or N,N′-diacetyl-L-cystine dimethyl ester)). Cysteine may be added to a cell culture by adding cell culture medium comprising the cysteine to the cell culture, or by adding a cell culture medium additive comprising the cysteine to the cell culture.

In some embodiments, ‘maintaining or adjusting the concentration of cysteine in the cell culture medium of the cell culture’ may comprise reducing the concentration of cysteine in the cell culture. In such embodiments, the methods may comprise adding cell culture medium comprising cysteine (e.g. in the form of monomeric cysteine, cystine, or a metabolic precursor or derivative of cysteine/cystine capable of metabolism to cysteine (e.g. as described hereinabove, e.g. N-acetyl-cysteine (NAC), N-acetyl-L-cysteine amide (NACA), S-sulfocysteine, N,N′-diacetyl-l-cystine or N,N′-diacetyl-L-cystine dimethyl ester)) at a concentration which is lower than the concentration of cysteine in the cell culture to which it is added, or adding cell culture medium (or an appropriate buffer, or water) lacking cysteine to the cell culture (thereby diluting the cysteine in the cell culture medium of the cell culture, and consequently reducing its concentration).

In some embodiments, ‘maintaining or adjusting the osmolality of the cell culture medium of the cell culture’ may comprise increasing the osmolality of the cell culture. In such embodiments, the methods may comprise adding an osmolality-increasing agent (e.g. as described hereinabove, e.g. an organic compound (e.g. a carbohydrate, e.g. glucose, trehalose or galactose) an inorganic compound (e.g. an inorganic salt, e.g. sodium chloride)) to the cell culture. An osmolality-increasing agent may be added to a cell culture by adding cell culture medium (or an appropriate buffer, or water) comprising the osmolality-increasing agent to the cell culture, or by adding a cell culture medium additive comprising the osmolality-increasing agent to the cell culture.

In some embodiments, ‘maintaining or adjusting the osmolality of the cell culture medium of the cell culture’ may comprise reducing the osmolality of the cell culture. In such embodiments, the methods may comprise adding cell culture medium (or an appropriate buffer, or water) having a lower osmolality than the osmolality of the cell culture to which it is added.

The present disclosure further provides the use of cell culture medium comprising certain concentrations of cysteine and/or having certain osmolality in methods relating to the production of KiH molecules.

The present disclosure provides the use of cell culture medium comprising cysteine at a concentration of less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM) to increase the yield of KiH molecules produced from cells comprising nucleic acid encoding the constituent polypeptides of the KiH molecule in cell culture. In some embodiments, the cell culture medium has an osmolality greater than 280 mOsmol/kg (e.g. >280 mOsmol/kg, e.g. >300 mOsmol/kg, e.g. ≥320 mOsmol/kg, e.g. ≥350 mOsmol/kg, e.g. ≥370 mOsmol/kg).

The present disclosure also provides the use of cell culture medium comprising cysteine at a concentration of less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM) to increase the proportion of KiH molecules among molecules produced from cells comprising nucleic acid encoding the constituent polypeptides of the KiH molecule in cell culture. In some embodiments, the cell culture medium has an osmolality greater than 280 mOsmol/kg (e.g. >280 mOsmol/kg and <450 mOsmol/kg, e.g. greater than 300 mOsmol/kg, e.g. >300 mOsmol/kg and <420 mOsmol/kg, e.g. ≥320 mOsmol/kg and ≤400 mOsmol/kg, e.g. ≥350 mOsmol/kg and 390 mOsmol/kg, e.g. about 370 mOsmol/kg).

The present disclosure provides the use of cell culture medium comprising cysteine at a concentration of less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM) to reduce the yield of an undesired product (e.g. a hole-hole molecule, a ½ hole molecule, a ½ knob molecule, or a knob-knob molecule) produced from cells comprising nucleic acid encoding the constituent polypeptides of a KiH molecule in cell culture. In some embodiments, the cell culture medium has an osmolality greater than 280 mOsmol/kg (e.g. >280 mOsmol/kg and <450 mOsmol/kg, e.g. greater than 300 mOsmol/kg, e.g. >300 mOsmol/kg and <420 mOsmol/kg, e.g. ≥320 mOsmol/kg and ≤400 mOsmol/kg, e.g. ≥350 mOsmol/kg and ≤390 mOsmol/kg, e.g. about 370 mOsmol/kg).

The present disclosure also provides the use of cell culture medium comprising cysteine at a concentration of less than 8 mM (e.g. ≥0.5 mM and <8 mM, e.g. less than 6 mM, e.g. ≥1 mM and <6 mM, e.g. ≥2 mM and <5 mM, e.g. ≥2.5 mM and <4.75 mM, e.g. about 3 mM or 4.5 mM) to reduce the proportion of an undesired product (e.g. a hole-hole molecule, a ½ hole molecule, a ½ knob molecule, or a knob-knob molecule) among molecules produced from cells comprising nucleic acid encoding the constituent polypeptides of a KiH molecule in cell culture. In some embodiments, the cell culture medium has an osmolality greater than 280 mOsmol/kg (e.g. >280 mOsmol/kg and <450 mOsmol/kg, e.g. greater than 300 mOsmol/kg, e.g. >300 mOsmol/kg and <420 mOsmol/kg, e.g. ≥320 mOsmol/kg and ≤400 mOsmol/kg, e.g. ≥350 mOsmol/kg and ≤390 mOsmol/kg, e.g. about 370 mOsmol/kg).

### Particular Exemplary Embodiments

The present disclosure provides a method for producing a molecule comprising a polypeptide complex formed by interaction between a first polypeptide comprising a CH3 region comprising a knob modification and a second polypeptide comprising a CH3 region comprising a hole modification, wherein the method comprises culturing cells comprising nucleic acid encoding the first and second polypeptides for the majority of the period of culture in cell culture medium: (a) comprising cysteine at a concentration of less than 6 mM, and (b) having an osmolality greater than 300 mOsmol/kg.

The present disclosure also provides a method for producing a molecule comprising a polypeptide complex formed by interaction between a first polypeptide comprising a CH3 region comprising a knob modification and a second polypeptide comprising a CH3 region comprising a hole modification, wherein the method comprises culturing cells comprising nucleic acid encoding the first and second polypeptides for the majority of the period of culture in cell culture medium: (a) comprising cysteine at a concentration of from 1 mM to 5 mM (e.g. about 3 mM or 4.5 mM), and (b) having an osmolality≥320 mOsmol/kg (e.g. ≥370 mOsmol/kg).

The present disclosure also provides a method for producing a molecule comprising a polypeptide complex formed by interaction between a first polypeptide comprising a CH3 region comprising a knob modification and a second polypeptide comprising a CH3 region comprising a hole modification, wherein the method comprises culturing cells comprising nucleic acid encoding the first and second polypeptides for the majority of the period of culture in cell culture medium: (a) comprising cysteine at a concentration of less than 6 mM, and (b) having an osmolality greater than 300 mOsmol/kg; and
wherein the method comprises seeding the cells in cell culture medium: (c) comprising cysteine at a concentration of from 1 mM to 5 mM (e.g. about 3 mM or 4.5 mM), and (d) having an osmolality of from 320 mOsmol/kg to 420 mOsmol/kg (e.g. about 370 mOsmol/kg).

The present disclosure also provides a method for increasing the yield of a molecule comprising a polypeptide complex formed by interaction between a first polypeptide comprising a CH3 region comprising a knob modification and a second polypeptide comprising a CH3 region comprising a hole modification produced from cells comprising nucleic acid encoding the first and second polypeptides, wherein the method comprises: (a) maintaining or adjusting the concentration of cysteine in the cell culture medium of the cell culture to less than 6 mM, and (b) maintaining or adjusting the osmolality of the cell culture medium of the cell culture to greater than 300 mOsmol/kg.

The present disclosure also provides a method for increasing the yield of a molecule comprising a polypeptide complex formed by interaction between a first polypeptide comprising a CH3 region comprising a knob modification and a second polypeptide comprising a CH3 region comprising a hole modification produced from cells comprising nucleic acid encoding the first and second polypeptides, wherein the method comprises: (a) maintaining or adjusting the concentration of cysteine in the cell culture medium of the cell culture to from 1 mM to 5 mM (e.g. about 3 mM or 4.5 mM), and (b) maintaining or adjusting the osmolality of the cell culture medium of the cell culture to ≥320 mOsmol/kg (e.g. ≥370 mOsmol/kg).

The present disclosure also provides a method for increasing the proportion of molecules comprising a polypeptide complex formed by interaction between a first polypeptide comprising a CH3 region comprising a knob modification and a second polypeptide comprising a CH3 region comprising a hole modification among molecules produced from cells comprising nucleic acid encoding the first and second polypeptides, wherein the method comprises: (a) maintaining or adjusting the concentration of cysteine in the cell culture medium of the cell culture to less than 6 mM, and (b) maintaining or adjusting the osmolality of the cell culture medium of the cell culture to greater than 300 mOsmol/kg.

The present disclosure also provides a method for increasing the proportion of molecules comprising a polypeptide complex formed by interaction between a first polypeptide comprising a CH3 region comprising a knob modification and a second polypeptide comprising a CH3 region comprising a hole modification among molecules produced from cells comprising nucleic acid encoding the first and second polypeptides, wherein the method comprises: (a) maintaining or adjusting the concentration of cysteine in the cell culture medium of the cell culture to from 1 mM to 5 mM (e.g. about 3 mM or 4.5 mM), and (b) maintaining or adjusting the osmolality of the cell culture medium of the cell culture to ≥320 mOsmol/kg (e.g ≥370 mOsmol/kg).

The present disclosure also provides a method for reducing the yield of a molecule comprising a polypeptide complex formed by interaction between polypeptides comprising a CH3 region comprising a hole modification among molecules produced from cells comprising nucleic acid encoding a first polypeptide comprising a CH3 region comprising a knob modification and a second polypeptide comprising a CH3 region comprising a hole modification in cell culture, wherein the method comprises: (a) maintaining or adjusting the concentration of cysteine in the cell culture medium of the cell culture to less than 6 mM, and (b) maintaining or adjusting the osmolality of the cell culture medium of the cell culture to greater than 300 mOsmol/kg.

The present disclosure also provides a method for reducing the yield of a molecule comprising a polypeptide complex formed by interaction between polypeptides comprising a CH3 region comprising a hole modification among molecules produced from cells comprising nucleic acid encoding a first polypeptide comprising a CH3 region comprising a knob modification and a second polypeptide comprising a CH3 region comprising a hole modification in cell culture, wherein the method comprises: (a) maintaining or adjusting the concentration of cysteine in the cell culture medium of the cell culture to from 1 mM to 5 mM (e.g. about 3 mM or 4.5 mM), and (b) maintaining or adjusting the osmolality of the cell culture medium of the cell culture to ≥320 mOsmol/kg (e.g. ≥370 mOsmol/kg).

The present disclosure also provides a method for reducing the proportion of molecules comprising a polypeptide complex formed by interaction between polypeptides comprising a CH3 region comprising a hole modification among molecules produced from cells comprising nucleic acid encoding a first polypeptide comprising a CH3 region comprising a knob modification and a second polypeptide comprising a CH3 region comprising a hole modification in cell culture, wherein the method comprises: (a) maintaining or adjusting the concentration of cysteine in the cell culture medium of the cell culture to less than 6 mM, and (b) maintaining or adjusting the osmolality of the cell culture medium of the cell culture to greater than 300 mOsmol/kg.

The present disclosure also provides a method for reducing the proportion of molecules comprising a polypeptide complex formed by interaction between polypeptides comprising a CH3 region comprising a hole modification among molecules produced from cells comprising nucleic acid encoding a first polypeptide comprising a CH3 region comprising a knob modification and a second polypeptide comprising a CH3 region comprising a hole modification in cell culture, wherein the method comprises: (a) maintaining or adjusting the concentration of cysteine in the cell culture medium of the cell culture to from 1 mM to 5 mM (e.g. about 3 mM or 4.5 mM), and (b) maintaining or adjusting the osmolality of the cell culture medium of the cell culture to ≥320 mOsmol/kg (e.g. ≥370 mOsmol/kg).

The present disclosure also provides the use of cell culture medium comprising: (a) cysteine at a concentration of less than 6 mM, and (b) having an osmolality greater than 300 mOsmol/kg, to increase the yield of a molecule comprising a polypeptide complex formed by interaction between a first polypeptide comprising a CH3 region comprising a knob modification and a second polypeptide comprising a CH3 region comprising a hole modification produced from cells comprising nucleic acid encoding the first and second polypeptides in cell culture.

The present disclosure also provides the use of cell culture medium comprising: (a) cysteine at a concentration of from 1 mM to 5 mM (e.g. about 3 mM or 4.5 mM), and (b) having an osmolality of from 320 mOsmol/kg to 420 mOsmol/kg (e.g. about 370 mOsmol/kg), to increase the yield of a molecule comprising a polypeptide complex formed by interaction between a first polypeptide comprising a CH3 region comprising a knob modification and a second polypeptide comprising a CH3 region comprising a hole modification produced from cells comprising nucleic acid encoding the first and second polypeptides in cell culture.

The present disclosure also provides the use of cell culture medium comprising: (a) cysteine at a concentration of less than 6 mM, and (b) having an osmolality greater than 300 mOsmol/kg, to increase the proportion of molecules comprising a polypeptide complex formed by interaction between a first polypeptide comprising a CH3 region comprising a knob modification and a second polypeptide comprising a CH3 region comprising a hole modification among molecules produced from cells comprising nucleic acid encoding the first and second polypeptides in cell culture.

The present disclosure also provides the use of cell culture medium comprising: (a) cysteine at a concentration of from 1 mM to 5 mM (e.g. about 3 mM or 4.5 mM), and (b) having an osmolality of from 320 mOsmol/kg to 420 mOsmol/kg (e.g. about 370 mOsmol/kg), to increase the proportion of molecules comprising a polypeptide complex formed by interaction between a first polypeptide comprising a CH3 region comprising a knob modification and a second polypeptide comprising a CH3 region comprising a hole modification among molecules produced from cells comprising nucleic acid encoding the first and second polypeptides in cell culture.

The present disclosure also provides the use of cell culture medium comprising: (a) cysteine at a concentration of less than 6 mM, and (b) having an osmolality greater than 300 mOsmol/kg, to reduce the yield of a molecule comprising a polypeptide complex formed by interaction between polypeptides comprising a CH3 region comprising a hole modification produced from cells comprising nucleic acid encoding a first polypeptide comprising a CH3 region comprising a knob modification and a second polypeptide comprising a CH3 region comprising a hole modification in cell culture.

The present disclosure also provides the use of cell culture medium comprising: (a) cysteine at a concentration of from 1 mM to 5 mM (e.g. about 3 mM or 4.5 mM), and (b) having an osmolality of from 320 mOsmol/kg to 420 mOsmol/kg (e.g. about 370 mOsmol/kg), to reduce the yield of a molecule comprising a polypeptide complex formed by interaction between polypeptides comprising a CH3 region comprising a hole modification produced from cells comprising nucleic acid encoding a first polypeptide comprising a CH3 region comprising a knob modification and a second polypeptide comprising a CH3 region comprising a hole modification in cell culture.

The present disclosure also provides the use of cell culture medium comprising: (a) cysteine at a concentration of less than 6 mM, and (b) having an osmolality greater than 300 mOsmol/kg, to reduce the proportion of molecules comprising a polypeptide complex formed by interaction between polypeptides comprising a CH3 region comprising a hole modification among molecules produced from cells comprising nucleic acid encoding a first polypeptide comprising a CH3 region comprising a knob modification and a second polypeptide comprising a CH3 region comprising a hole modification in cell culture.

The present disclosure also provides the use of cell culture medium comprising: (a) cysteine at a concentration of from 1 mM to 5 mM (e.g. about 3 mM or 4.5 mM), and (b) having an osmolality of from 320 mOsmol/kg to 420 mOsmol/kg (e.g. about 370 mOsmol/kg), to reduce the proportion of molecules comprising a polypeptide complex formed by interaction between polypeptides comprising a CH3 region comprising a hole modification among molecules produced from cells comprising nucleic acid encoding a first polypeptide comprising a CH3 region comprising a knob modification and a second polypeptide comprising a CH3 region comprising a hole modification in cell culture.

### Increased Yields and Proportions of KiH Molecules, and Reduced Yields and Proportions of Undesired Products

As explained herein, culture of cells comprising nucleic acid encoding the constituent polypeptides of KiH molecules in accordance with the culture conditions described herein increases the yield of KiH molecules and/or increases the proportion of KiH molecules among the molecules produced by the cells in culture, and/or reduces the yield of undesired products (e.g. hole-hole molecules, ½ hole molecules, ½ knob molecules, and/or knob-knob molecules) and/or the reduces proportion of undesired products (e.g. hole-hole molecules, ½ hole molecules, ½ knob molecules, and/or knob-knob molecules) among the molecules produced by the cells in culture.

It will be appreciated that the ‘yield’ of a given molecule refers to the amount/quantity of the given molecule produced. It will similarly be appreciated that the ‘proportion’ of a given molecule among a plurality of different kinds of molecule refers to the fraction/percentage of the given molecule within the total amount/quantity of the different kinds of molecule produced. By way of illustration, within a population of molecules comprising KiH molecules and e.g. hole-hole molecules and ½ knob molecules, the proportion of KiH molecule is the fraction/percentage of the total amount/quantity of the molecules of the population of molecules that are KiH molecules.

An ‘increased’ yield/proportion of KiH molecules may be increased relative to the yield/proportion of KiH molecules obtained by culture according to a reference method for producing KiH molecules from cells comprising nucleic acid encoding the constituent polypeptides of KiH molecules in culture. Similarly, a ‘reduced’ yield/proportion of an undesired product (e.g. a hole-hole molecule, ½ hole molecule, ½ knob molecule, or a knob-knob molecule) may be reduced relative to the yield/proportion of the relevant molecule obtained by culture according to a reference method for producing KiH molecules from cells comprising nucleic acid encoding the constituent polypeptides of KiH molecules in culture.

A reference method for producing KiH molecules from cells comprising nucleic acid encoding the constituent polypeptides of KiH molecules in culture may be a method in which the cells are not cultured in cell culture medium comprising cysteine at a concentration of less than 8 mM for the majority of the period of culture. For example, in the reference method the cells may be cultured in cell culture medium comprising cysteine at a concentration of ≥8 mM for the majority of the period of culture. The reference method may be a method in which the cells are not cultured in cell culture medium comprising cysteine at a concentration of less than 6 mM for the majority of the period of culture. For example, in the reference method the cells may be cultured in cell culture medium comprising cysteine at a concentration of ≥6 mM for the majority of the period of culture.

The reference method may be a method in which the cells are not cultured in cell culture medium having an osmolality greater than 280 mOsmol/kg for the majority of the period of culture. For example, in the reference method the cells may be cultured in cell culture medium having an osmolality≤280 mOsmol/kg for the majority of the period of culture. The reference method may be a method in which the cells are not cultured in cell culture medium having an osmolality greater than 300 mOsmol/kg for the majority of the period of culture. For example, in the reference method the cells may be cultured in cell culture medium having an osmolality 300 mOsmol/kg for the majority of the period of culture. The reference method may be a method in which the cells are not seeded in cell culture medium having an osmolality greater than 280 mOsmol/kg. For example, in the reference method the cells may be seeded in cell culture medium having an osmolality 280 mOsmol/kg. The reference method may be a method in which the cells are not seeded in cell culture medium having an osmolality greater than 300 mOsmol/kg. For example, in the reference method the cells may be seeded in cell culture medium having an osmolality 300 mOsmol/kg.

An ‘increased’ yield/proportion of KiH molecules relative to the yield/proportion of KiH molecules obtained by culture according to a reference method for producing KiH molecules from cells comprising nucleic acid encoding the constituent polypeptides of KiH molecules in culture is greater than 1 times (e.g. may be ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times) the yield/proportion of KiH molecules obtained by culture according to the reference method.

A ‘reduced’ yield/proportion of an undesired product (e.g. a hole-hole molecule, a ½ hole molecule, a ½ knob molecule, or a knob-knob molecule) relative to the yield/proportion of the relevant molecule obtained by culture according to a reference method for producing KiH molecules from cells comprising nucleic acid encoding the constituent polypeptides of KiH molecules in culture is less than 1 times (e.g. may be ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, 0.2 times, ≤0.15 times, ≤0.1 times, ≤0.05 times, or ≤0.01 times) the yield/proportion of the relevant molecule obtained by culture according to the reference method.

### Sequence Identity

As used herein, ‘sequence identity’ refers to the percent of nucleotides/amino acid residues in a subject sequence that are identical to nucleotides/amino acid residues in a reference sequence, after aligning the sequences and, if necessary, introducing gaps, to achieve the maximum percent sequence identity between the sequences. Pairwise and multiple sequence alignment for the purposes of determining percent sequence identity between two or more amino acid or nucleic acid sequences can be achieved in various ways known to a person of skill in the art, for instance, using publicly available computer software such as ClustalOmega (Söding, J. Bioinformatics (2005) 21:951-960), T-coffee (Notredame et al. J Mol Biol (2000) 302:205-217), Kalign (Lassmann and Sonnhammer BMC Bioinformatics (2005) 6(298)) and MAFFT (Katoh and Standley Molecular Biology and Evolution (2013) 30(4):772-780) software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | Human IgG1 | ASTKGPSVFPLAPSSKSTSGGTAALGCLVKDYFPEPVTV |
| | constant region | SWNSGALTSGVHTFPAVLQSSGLYSLSSVVTVPSSSLGT |
| | (IGHG1; | QTYICNVNHKPSNTKVDKKVEPKSCDKTHTCPPCPAPEL |
| | UniProt: P01857-1, | LGGPSVFLFPPKPKDTLMISRTPEVTCVVVDVSHEDPEV |
| | v1) | KFNWYVDGVEVHNAKTKPREEQYNSTYRVVSVLTVLH |
| | | QDWLNGKEYKCKVSNKALPAPIEKTISKAKGQPREPQV |
| | | YTLPPSRDELTKNQVSLTCLVKGFYPSDIAVEWESNGQP |
| | | ENNYKTTPPVLDSDGSFFLYSKLTVDKSRWQQGNVFSC |
| | | SVMHEALHNHYTQKSLSLSPGK |
| 2 | CH1 IgG1 | ASTKGPSVFPLAPSSKSTSGGTAALGCLVKDYFPEPVTV |
| | (positions 1-98 of | SWNSGALTSGVHTFPAVLQSSGLYSLSSVVTVPSSSLGT |
| | P01857-1, v1) | QTYICNVNHKPSNTKVDKKV |
| 3 | Hinge IgG1 | EPKSCDKTHTCPPCP |
| | (positions 99-113 | |
| | of P01857-1, v1) | |
| 4 | CH2 IgG1 | APELLGGPSVFLFPPKPKDTLMISRTPEVTCVVVDVSHE |
| | (positions 114-223 | DPEVKFNWYVDGVEVHNAKTKPREEQYNSTYRVVSVL |
| | of P01857-1, v1) | TVLHQDWLNGKEYKCKVSNKALPAPIEKTISKAK |
| 5 | CH3 IgG1 | GQPREPQVYTLPPSRDELTKNQVSLTCLVKGFYPSDIAV |
| | (positions 224-330 | EWESNGQPENNYKTTPPVLDSDGSFFLYSKLTVDKSRW |
| | of P01857-1, v1) | QQGNVFSCSVMHEALHNHYTQKSLSLSPGK |
| 6 | Human IgG1 | ASTKGPSVFPLAPSSKSTSGGTAALGCLVKDYFPEPVTV |
| | constant region | SWNSGALTSGVHTFPAVLQSSGLYSLSSVVTVPSSSLGT |
| | G1m3 allotype | QTYICNVNHKPSNTKVDKRVEPKSCDKTHTCPPCPAPEL |
| | (K214R, D356E | LGGPSVFLFPPKPKDTLMISRTPEVTCVVVDVSHEDPEV |
| | and L358M (EU | KFNWYVDGVEVHNAKTKPREEQYNSTYRVVSVLTVLH |
| | numbering) | QDWLNGKEYKCKVSNKALPAPIEKTISKAKGQPREPQV |
| | relative to P01857- | YTLPPSREEMTKNQVSLTCLVKGFYPSDIAVEWESNGQP |
| | 1) | ENNYKTTPPVLDSDGSFFLYSKLTVDKSRWQQGNVFSC |
| | | SVMHEALHNHYTQKSLSLSPGK |
| 7 | CH1 IgG1 G1m3 | ASTKGPSVFPLAPSSKSTSGGTAALGCLVKDYFPEPVTV |
| | allotype | SWNSGALTSGVHTFPAVLQSSGLYSLSSVVTVPSSSLGT |
| | | QTYICNVNHKPSNTKVDKRV |
| 8 | CH3 IgG1 G1m3 | GQPREPQVYTLPPSREEMTKNQVSLTCLVKGFYPSDIAV |
| | allotype | EWESNGQPENNYKTTPPVLDSDGSFFLYSKLTVDKSRW |
| | | QQGNVFSCSVMHEALHNHYTQKSLSLSPGK |
| 9 | Human IgG2 CH3 | GQPREPQVYTLPPSREEMTKNQVSLTCLVKGFYPSDISV |
| | domain | EWESNGQPENNYKTTPPMLDSDGSFFLYSKLTVDKSRW |
| | | QQGNVFSCSVMHEALHNHYTQKSLSLSPGK |
| 10 | Human IgG3 CH3 | GQPREPQVYTLPPSREEMTKNQVSLTCLVKGFYPSDIAV |
| | domain | EWESSGQPENNYNTTPPMLDSDGSFFLYSKLTVDKSRW |
| | | QQGNIFSCSVMHEALHNRFTQKSLSLSPGK |
| 11 | Human IgG4 CH3 | GQPREPQVYTLPPSQEEMTKNQVSLTCLVKGFYPSDIAV |
| | domain | EWESNGQPENNYKTTPPVLDSDGSFFLYSRLTVDKSRW |
| | | QEGNVFSCSVMHEALHNHYTQKSLSLSLGK |
| 12 | hIgG1 (G1m1) | GQPREPQVYTLPPSRDELTKNQVSLWCLVKGFYPSDIAV |
| | CH3 T366W | EWESNGQPENNYKTTPPVLDSDGSFFLYSKLTVDKSRW |
| | | QQGNVFSCSVMHEALHNHYTQKSLSLSPGK |
| 13 | hIgG1 (G1m3) | GQPREPQVYTLPPSREEMTKNQVSLWCLVKGFYPSDIA |
| | CH3 T366W | VEWESNGQPENNYKTTPPVLDSDGSFFLYSKLTVDKSR |
| | | WQQGNVFSCSVMHEALHNHYTQKSLSLSPGK |
| 14 | hIgG2 CH3 | GQPREPQVYTLPPSREEMTKNQVSLWCLVKGFYPSDISV |
| | T366W | EWESNGQPENNYKTTPPMLDSDGSFFLYSKLTVDKSRW |
| | | QQGNVFSCSVMHEALHNHYTQKSLSLSPGK |
| 15 | hIgG3 CH3 | GQPREPQVYTLPPSREEMTKNQVSLWCLVKGFYPSDIA |
| | T366W | VEWESSGQPENNYNTTPPMLDSDGSFFLYSKLTVDKSR |
| | | WQQGNIFSCSVMHEALHNRFTQKSLSLSPGK |
| 16 | hIgG4 CH3 | GQPREPQVYTLPPSQEEMTKNQVSLWCLVKGFYPSDIA |
| | T366W | VEWESNGQPENNYKTTPPVLDSDGSFFLYSRLTVDKSR |
| | | WQEGNVFSCSVMHEALHNHYTQKSLSLSLGK |
| 17 | hIgG1 (G1m1) | GQPREPQVYTLPPCRDELTKNQVSLWCLVKGFYPSDIAV |
| | CH3 T366W, | EWESNGQPENNYKTTPPVLDSDGSFFLYSKLTVDKSRW |
| | S354C | QQGNVFSCSVMHEALHNHYTQKSLSLSPGK |
| 18 | hIgG1 (G1m3) | GQPREPQVYTLPPCREEMTKNQVSLWCLVKGFYPSDIA |
| | CH3 T366W, | VEWESNGQPENNYKTTPPVLDSDGSFFLYSKLTVDKSR |
| | S354C | WQQGNVFSCSVMHEALHNHYTQKSLSLSPGK |
| 19 | hIgG2 CH3 | GQPREPQVYTLPPCREEMTKNQVSLWCLVKGFYPSDISV |
| | T366W, S354C | EWESNGQPENNYKTTPPMLDSDGSFFLYSKLTVDKSRW |
| | | QQGNVFSCSVMHEALHNHYTQKSLSLSPGK |
| 20 | hIgG3 CH3 | GQPREPQVYTLPPCREEMTKNQVSLWCLVKGFYPSDIA |
| | T366W, S354C | VEWESSGQPENNYNTTPPMLDSDGSFFLYSKLTVDKSR |
| | | WQQGNIFSCSVMHEALHNRFTQKSLSLSPGK |
| 21 | hIgG4 CH3 | GQPREPQVYTLPPCQEEMTKNQVSLWCLVKGFYPSDIA |
| | T366W, S354C | VEWESNGQPENNYKTTPPVLDSDGSFFLYSRLTVDKSR |
| | | WQEGNVFSCSVMHEALHNHYTQKSLSLSLGK |
| 22 | hIgG1 (G1m1) | GQPREPQVYTLPPSRDELTKNQVSLSCAVKGFYPSDIAV |
| | CH3 T366S, | EWESNGQPENNYKTTPPVLDSDGSFFLVSKLTVDKSRW |
| | L368A, Y407V | QQGNVFSCSVMHEALHNHYTQKSLSLSPGK |
| 23 | hIgG1 (G1m3) | GQPREPQVYTLPPSREEMTKNQVSLSCAVKGFYPSDIAV |
| | CH3 T366S, | EWESNGQPENNYKTTPPVLDSDGSFFLVSKLTVDKSRW |
| | L368A, Y407V | QQGNVFSCSVMHEALHNHYTQKSLSLSPGK |
| 24 | hIgG2 CH3 T366S, | GQPREPQVYTLPPSREEMTKNQVSLSCAVKGFYPSDISV |
| | L368A, Y407V | EWESNGQPENNYKTTPPMLDSDGSFFLVSKLTVDKSRW |
| | | QQGNVFSCSVMHEALHNHYTQKSLSLSPGK |
| 25 | hIgG3 CH3 T366S, | GQPREPQVYTLPPSREEMTKNQVSLSCAVKGFYPSDIAV |
| | L368A, Y407V | EWESSGQPENNYNTTPPMLDSDGSFFLVSKLTVDKSRW |
| | | QQGNIFSCSVMHEALHNRFTQKSLSLSPGK |
| 26 | hIgG4 CH3 T366S, | GQPREPQVYTLPPSQEEMTKNQVSLSCAVKGFYPSDIAV |
| | L368A, Y407V | EWESNGQPENNYKTTPPVLDSDGSFFLVSRLTVDKSRW |
| | | QEGNVFSCSVMHEALHNHYTQKSLSLSLGK |
| 27 | hIgG1 (G1m1) | GQPREPQVCTLPPSRDELTKNQVSLSCAVKGFYPSDIAV |
| | CH3 T366S, | EWESNGQPENNYKTTPPVLDSDGSFFLVSKLTVDKSRW |
| | L368A, Y407V, | QQGNVFSCSVMHEALHNHYTQKSLSLSPGK |
| | Y349C | |
| 28 | hIgG1 (G1m3) | GQPREPQVCTLPPSREEMTKNQVSLSCAVKGFYPSDIAV |
| | CH3 T366S, | EWESNGQPENNYKTTPPVLDSDGSFFLVSKLTVDKSRW |
| | L368A, Y407V, | QQGNVFSCSVMHEALHNHYTQKSLSLSPGK |
| | Y349C | |
| 29 | hIgG2 CH3 T366S, | GQPREPQVCTLPPSREEMTKNQVSLSCAVKGFYPSDISV |
| | L368A, Y407V, | EWESNGQPENNYKTTPPMLDSDGSFFLVSKLTVDKSRW |
| | Y349C | QQGNVFSCSVMHEALHNHYTQKSLSLSPGK |
| 30 | hIgG3 CH3 T366S, | GQPREPQVCTLPPSREEMTKNQVSLSCAVKGFYPSDIAV |
| | L368A, Y407V, | EWESSGQPENNYNTTPPMLDSDGSFFLVSKLTVDKSRW |
| | Y349C | QQGNIFSCSVMHEALHNRFTQKSLSLSPGK |
| 31 | hIgG4 CH3 T366S, | GQPREPQVCTLPPSQEEMTKNQVSLSCAVKGFYPSDIAV |
| | L368A, Y407V, | EWESNGQPENNYKTTPPVLDSDGSFFLVSRLTVDKSRW |
| | Y349C | QEGNVFSCSVMHEALHNHYTQKSLSLSLGK |
| 32 | Human IgG2 CH2 | APPVAGPSVFLFPPKPKDTLMISRTPEVTCVVVDVSHED |
| | domain | PEVQFNWYVDGVEVHNAKTKPREEQFNSTFRVVSVLTV |
| | | VHQDWLNGKEYKCKVSNKGLPAPIEKTISKTK |
| 33 | Human IgG3 CH2 | APELLGGPSVFLFPPKPKDTLMISRTPEVTCVVVDVSHE |
| | domain | DPEVQFKWYVDGVEVHNAKTKPREEQYNSTFRVVSVL |
| | | TVLHQDWLNGKEYKCKVSNKALPAPIEKTISKTK |
| 34 | Human IgG4 CH2 | APEFLGGPSVFLFPPKPKDTLMISRTPEVTCVVVDVSQED |
| | domain | PEVQFNWYVDGVEVHNAKTKPREEQFNSTYRVVSVLT |
| | | VLHQDWLNGKEYKCKVSNKGLPSSIEKTISKAK |
| 35 | hIgG1 CH2-CH3 | APELLGGPSVFLFPPKPKDTLMISRTPEVTCVVVDVSHE |
| | (G1m1) | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSRDELTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| 36 | hIgG1 CH2-CH3 | APELLGGPSVFLFPPKPKDTLMISRTPEVTCVVVDVSHE |
| | (G1m3) | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSREEMTKNQVSLTCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| 37 | hIgG1 (G1m1) | APELLGGPSVFLFPPKPKDTLMISRTPEVTCVVVDVSHE |
| | CH2-CH3 T366W | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSRDELTKNQVSLWCLVKGFYPSDIAVEWES |
| | | NGQPENNYKTTPPVLDSDGSFFLYSKLTVDKSRWQQGN |
| | | VFSCSVMHEALHNHYTQKSLSLSPGK |
| 38 | hIgG1 (G1m3) | APELLGGPSVFLFPPKPKDTLMISRTPEVTCVVVDVSHE |
| | CH2-CH3 T366W | DPEVKFNWYVDGVEVHNAKTKPREEQYNSTYRVVSVL |
| | | TVLHQDWLNGKEYKCKVSNKALPAPIEKTISKAKGQPR |
| | | EPQVYTLPPSREEMTKNQVSLWCLVKGFYPSDIAVEWE |
| | | SNGQPENNYKTTPPVLDSDGSFFLYSKLTVDKSRWQQG |
| | | NVFSCSVMHEALHNHYTQKSLSLSPGK |
| 39 | hIgG2 CH2-CH3 | APPVAGPSVFLFPPKPKDTLMISRTPEVTCVVVDVSHED |
| | T366W | PEVQFNWYVDGVEVHNAKTKPREEQFNSTFRVVSVLTV |
| | | VHQDWLNGKEYKCKVSNKGLPAPIEKTISKTKGQPREP |
| | | QVYTLPPSREEMTKNQVSLWCLVKGFYPSDISVEWESN |
| | | GQPENNYKTTPPMLDSDGSFFLYSKLTVDKSRWQQGNV |
| | | FSCSVMHEALHNHYTQKSLSLSPGK |
| 40 | hIgG3 CH2-CH3 | APELLGGPSVFLFPPKPKDTLMISRTPEVTCVVVDVSHE |
| | T366W | DPEVQFKWYVDGVEVHNAKTKPREEQYNSTFRVVSVL |
| | | TVLHQDWLNGKEYKCKVSNKALPAPIEKTISKTKGQPR |
| | | EPQVYTLPPSREEMTKNQVSLWCLVKGFYPSDIAVEWE |
| | | SSGQPENNYNTTPPMLDSDGSFFLYSKLTVDKSRWQQG |
| | | NIFSCSVMHEALHNRFTQKSLSLSPGK |
| 41 | hIgG4 CH2-CH3 | APEFLGGPSVFLFPPKPKDTLMISRTPEVTCVVVDVSQED |
| | T366W | PEVQFNWYVDGVEVHNAKTKPREEQFNSTYRVVSVLT |
| | | VLHQDWLNGKEYKCKVSNKGLPSSIEKTISKAKGQPREP |
| | | QVYTLPPSQEEMTKNQVSLWCLVKGFYPSDIAVEWESN |
| | | GQPENNYKTTPPVLDSDGSFFLYSRLTVDKSRWQEGNV |
| | | FSCSVMHEALHNHYTQKSLSLSLGK |
| 42 | hIgG1 (G1m1) | APELLGGPSVFLFPPKPKDTLMISRTPEVTCVVVDVSHE |
| | CH2-CH3 T366W, | DPEVKFNWYVDGVEVHNAKTKPREEQYNSTYRVVSVL |
| | S354C | TVLHQDWLNGKEYKCKVSNKALPAPIEKTISKAKGQPR |
| | | EPQVYTLPPCRDELTKNQVSLWCLVKGFYPSDIAVEWE |
| | | SNGQPENNYKTTPPVLDSDGSFFLYSKLTVDKSRWQQG |
| | | NVFSCSVMHEALHNHYTQKSLSLSPGK |
| 43 | hIgG1 (G1m3) | APELLGGPSVFLFPPKPKDTLMISRTPEVTCVVVDVSHE |
| | CH2-CH3 T366W, | DPEVKFNWYVDGVEVHNAKTKPREEQYNSTYRVVSVL |
| | S354C | TVLHQDWLNGKEYKCKVSNKALPAPIEKTISKAKGQPR |
| | | EPQVYTLPPCREEMTKNQVSLWCLVKGFYPSDIAVEWE |
| | | SNGQPENNYKTTPPVLDSDGSFFLYSKLTVDKSRWQQG |
| | | NVFSCSVMHEALHNHYTQKSLSLSPGK |
| 44 | hIgG2 CH2-CH3 | APPVAGPSVFLFPPKPKDTLMISRTPEVTCVVVDVSHED |
| | T366W, S354C | PEVQFNWYVDGVEVHNAKTKPREEQFNSTFRVVSVLTV |
| | | VHQDWLNGKEYKCKVSNKGLPAPIEKTISKTKGQPREP |
| | | QVYTLPPCREEMTKNQVSLWCLVKGFYPSDISVEWESN |
| | | GQPENNYKTTPPMLDSDGSFFLYSKLTVDKSRWQQGNV |
| | | FSCSVMHEALHNHYTQKSLSLSPGK |
| 45 | hIgG3 CH2-CH3 | APELLGGPSVFLFPPKPKDTLMISRTPEVTCVVVDVSHE |
| | T366W, S354C | DPEVQFKWYVDGVEVHNAKTKPREEQYNSTFRVVSVL |
| | | TVLHQDWLNGKEYKCKVSNKALPAPIEKTISKTKGQPR |
| | | EPQVYTLPPCREEMTKNQVSLWCLVKGFYPSDIAVEWE |
| | | SSGQPENNYNTTPPMLDSDGSFFLYSKLTVDKSRWQQG |
| | | NIFSCSVMHEALHNRFTQKSLSLSPGK |
| 46 | hIgG4 CH2-CH3 | APEFLGGPSVFLFPPKPKDTLMISRTPEVTCVVVDVSQED |
| | T366W, S354C | PEVQFNWYVDGVEVHNAKTKPREEQFNSTYRVVSVLT |
| | | VLHQDWLNGKEYKCKVSNKGLPSSIEKTISKAKGQPREP |
| | | QVYTLPPCQEEMTKNQVSLWCLVKGFYPSDIAVEWESN |
| | | GQPENNYKTTPPVLDSDGSFFLYSRLTVDKSRWQEGNV |
| | | FSCSVMHEALHNHYTQKSLSLSLGK |
| 47 | hIgG1 (G1m1) | APELLGGPSVFLFPPKPKDTLMISRTPEVTCVVVDVSHE |
| | CH2-CH3 T366S, | DPEVKFNWYVDGVEVHNAKTKPREEQYNSTYRVVSVL |
| | L368A, Y407V | TVLHQDWLNGKEYKCKVSNKALPAPIEKTISKAKGQPR |
| | | EPQVYTLPPSRDELTKNQVSLSCAVKGFYPSDIAVEWES |
| | | NGQPENNYKTTPPVLDSDGSFFLVSKLTVDKSRWQQGN |
| | | VFSCSVMHEALHNHYTQKSLSLSPGK |
| 48 | hIgG1 (G1m3) | GQPREPQVYTLPPSREEMTKNQVSLSCAVKGFYPSDIAV |
| | CH2-CH3 T366S, | EWESNGQPENNYKTTPPVLDSDGSFFLVSKLTVDKSRW |
| | L368A, Y407V | QQGNVFSCSVMHEALHNHYTQKSLSLSPGK |
| 49 | hIgG2 CH2-CH3 | APPVAGPSVFLFPPKPKDTLMISRTPEVTCVVVDVSHED |
| | T366S, L368A, | PEVQFNWYVDGVEVHNAKTKPREEQFNSTFRVVSVLTV |
| | Y407V | VHQDWLNGKEYKCKVSNKGLPAPIEKTISKTKGQPREP |
| | | QVYTLPPSREEMTKNQVSLSCAVKGFYPSDISVEWESNG |
| | | QPENNYKTTPPMLDSDGSFFLVSKLTVDKSRWQQGNVF |
| | | SCSVMHEALHNHYTQKSLSLSPGK |
| 50 | hIgG3 CH2-CH3 | APELLGGPSVFLFPPKPKDTLMISRTPEVTCVVVDVSHE |
| | T366S, L368A, | DPEVQFKWYVDGVEVHNAKTKPREEQYNSTFRVVSVL |
| | Y407V | TVLHQDWLNGKEYKCKVSNKALPAPIEKTISKTKGQPR |
| | | EPQVYTLPPSREEMTKNQVSLSCAVKGFYPSDIAVEWES |
| | | SGQPENNYNTTPPMLDSDGSFFLVSKLTVDKSRWQQGN |
| | | IFSCSVMHEALHNRFTQKSLSLSPGK |
| 51 | hIgG4 CH2-CH3 | APEFLGGPSVFLFPPKPKDTLMISRTPEVTCVVVDVSQED |
| | T366S, L368A, | PEVQFNWYVDGVEVHNAKTKPREEQFNSTYRVVSVLT |
| | Y407V | VLHQDWLNGKEYKCKVSNKGLPSSIEKTISKAKGQPREP |
| | | QVYTLPPSQEEMTKNQVSLSCAVKGFYPSDIAVEWESN |
| | | GQPENNYKTTPPVLDSDGSFFLVSRLTVDKSRWQEGNV |
| | | FSCSVMHEALHNHYTQKSLSLSLGK |
| 52 | hIgG1 (G1m1) | APELLGGPSVFLFPPKPKDTLMISRTPEVTCVVVDVSHE |
| | CH2-CH3 T366S, | DPEVKFNWYVDGVEVHNAKTKPREEQYNSTYRVVSVL |
| | L368A, Y407V, | TVLHQDWLNGKEYKCKVSNKALPAPIEKTISKAKGQPR |
| | Y349C | EPQVCTLPPSRDELTKNQVSLSCAVKGFYPSDIAVEWES |
| | | NGQPENNYKTTPPVLDSDGSFFLVSKLTVDKSRWQQGN |
| | | VFSCSVMHEALHNHYTQKSLSLSPGK |
| 53 | hIgG1 (G1m3) | APELLGGPSVFLFPPKPKDTLMISRTPEVTCVVVDVSHE |
| | CH2-CH3 T366S, | DPEVKFNWYVDGVEVHNAKTKPREEQYNSTYRVVSVL |
| | L368A, Y407V, | TVLHQDWLNGKEYKCKVSNKALPAPIEKTISKAKGQPR |
| | Y349C | EPQVCTLPPSREEMTKNQVSLSCAVKGFYPSDIAVEWES |
| | | NGQPENNYKTTPPVLDSDGSFFLVSKLTVDKSRWQQGN |
| | | VFSCSVMHEALHNHYTQKSLSLSPGK |
| 54 | hIgG2 CH2-CH3 | APPVAGPSVFLFPPKPKDTLMISRTPEVTCVVVDVSHED |
| | T366S, L368A, | PEVQFNWYVDGVEVHNAKTKPREEQFNSTFRVVSVLTV |
| | Y407V, Y349C | VHQDWLNGKEYKCKVSNKGLPAPIEKTISKTKGQPREP |
| | | QVCTLPPSREEMTKNQVSLSCAVKGFYPSDISVEWESNG |
| | | QPENNYKTTPPMLDSDGSFFLVSKLTVDKSRWQQGNVF |
| | | SCSVMHEALHNHYTQKSLSLSPGK |
| 55 | hIgG3 CH2-CH3 | APELLGGPSVFLFPPKPKDTLMISRTPEVTCVVVDVSHE |
| | T366S, L368A, | DPEVQFKWYVDGVEVHNAKTKPREEQYNSTFRVVSVL |
| | Y407V, Y349C | TVLHQDWLNGKEYKCKVSNKALPAPIEKTISKTKGQPR |
| | | EPQVCTLPPSREEMTKNQVSLSCAVKGFYPSDIAVEWES |
| | | SGQPENNYNTTPPMLDSDGSFFLVSKLTVDKSRWQQGN |
| | | IFSCSVMHEALHNRFTQKSLSLSPGK |
| 56 | hIgG4 CH2-CH3 | APEFLGGPSVFLFPPKPKDTLMISRTPEVTCVVVDVSQED |
| | T366S, L368A, | PEVQFNWYVDGVEVHNAKTKPREEQFNSTYRVVSVLT |
| | Y407V, Y349C | VLHQDWLNGKEYKCKVSNKGLPSSIEKTISKAKGQPREP |
| | | QVCTLPPSQEEMTKNQVSLSCAVKGFYPSDIAVEWESN |
| | | GQPENNYKTTPPVLDSDGSFFLVSRLTVDKSRWQEGNV |
| | | FSCSVMHEALHNHYTQKSLSLSLGK |
| 57 | hu 4-1BBL (50- | ACPWAVSGARASPGSAASPRLREGPELSPDDPAGLLDLR |
| | 254) | QGMFAQLVAQNVLLIDGPLSWYSDPGLAGVSLTGGLSY |
| | | KEDTKELVVAKAGVYYVFFQLELRRVVAGEGSGSVSLA |
| | | LHLQPLRSAAGAAALALTVDLPPASSEARNSAFGFQGRL |
| | | LHLSAGQRLGVHLHTEARARHAWQLTQGATVLGLFRV |
| | | TPEIPAGLPSPRSE |
| 58 | Human (hu) 4-1 | REGPELSPDD PAGLLDLRQG MFAQLVAQNV |
| | BBL (71-254) | LLIDGPLSWY SDPGLAGVSL TGGLSYKEDT |
| | | KELVVAKAGV YYVFFQLELR RVVAGEGSGS |
| | | VSLALHLQPL RSAAGAAALA LTVDLPPASS |
| | | EARNSAFGFQ GRLLHLSAGQ RLGVHLHTEA |
| | | RARHAWQLTQ GATVLGLFRV TPEIPAGLPS PRSE |
| 59 | Human (hu) 4-1 | LDLRQGMFAQ LVAQNVLLID GPLSWYSDPG |
| | BBL (85-254) | LAGVSLTGGL SYKEDTKELV VAKAGVYYVF |
| | | FQLELRRVVA GEGSGSVSLA LHLQPLRSAA |
| | | GAAALALTVD LPPASSEARN SAFGFQGRLL |
| | | HLSAGQRLGV HLHTEARARH AWQLTQGATV |
| | | LGLFRVTPEI PAGLPSPRSE |
| 60 | Human (hu) 4- | DPAGLLDLRQ GMFAQLVAQN VLLIDGPLSW |
| | 1BBL (80-254) | YSDPGLAGVS LTGGLSYKED TKELVVAKAG |
| | | VYYVFFQLEL RRVVAGEGSG SVSLALHLQP |
| | | LRSAAGAAAL ALTVDLPPAS SEARNSAFGF |
| | | QGRLLHLSAG QRLGVHLHTE ARARHAWQLT |
| | | QGATVLGLFR VTPEIPAGLP SPRSE |
| 61 | Human (hu) 4- | PWAVSGARAS PGSAASPRLR EGPELSPDDP |
| | 1BBL (52-254) | AGLLDLRQGM FAQLVAQNVL LIDGPLSWYS |
| | | DPGLAGVSLT GGLSYKEDTK ELVVAKAGVY |
| | | YVFFQLELRR VVAGEGSGSV SLALHLQPLR |
| | | SAAGAAALAL TVDLPPASSE ARNSAFGFQG |
| | | RLLHLSAGQR LGVHLHTEAR ARHAWQLTQG |
| | | ATVLGLFRVT PEIPAGLPSP RSE |
| 62 | Human (hu) 4- | REGPELSPDD PAGLLDLRQG MFAQLVAQNV |
| | 1BBL (71-248) | LLIDGPLSWY SDPGLAGVSL TGGLSYKEDT |
| | | KELVVAKAGV YYVFFQLELR RVVAGEGSGS |
| | | VSLALHLQPL RSAAGAAALA LTVDLPPASS |
| | | EARNSAFGFQ GRLLHLSAGQ RLGVHLHTEA |
| | | RARHAWQLTQ GATVLGLFRV TPEIPAGL |
| 63 | Human (hu) 4- | LDLRQGMFAQ LVAQNVLLID GPLSWYSDPG |
| | 1BBL (85-248) | LAGVSLTGGL SYKEDTKELV VAKAGVYYVF |
| | | FQLELRRVVA GEGSGSVSLA LHLQPLRSAA |
| | | GAAALALTVD LPPASSEARN SAFGFQGRLL |
| | | HLSAGQRLGV HLHTEARARH AWQLTQGATV |
| | | LGLFRVTPEI PAGL |
| 64 | Human (hu) 4- | DPAGLLDLRQ GMFAQLVAQN VLLIDGPLSW |
| | 1BBL (80-248) | YSDPGLAGVS LTGGLSYKED TKELVVAKAG |
| | | VYYVFFQLEL RRVVAGEGSG SVSLALHLQP |
| | | LRSAAGAAAL ALTVDLPPAS SEARNSAFGF |
| | | QGRLLHLSAG QRLGVHLHTE ARARHAWQLT |
| | | QGATVLGLFR VTPEIPAGL |
| 65 | Human (hu) 4- | PWAVSGARAS PGSAASPRLR EGPELSPDDP |
| | 1BBL (52-248) | AGLLDLRQGM FAQLVAQNVL LIDGPLSWYS |
| | | DPGLAGVSLT GGLSYKEDTK ELVVAKAGVY |
| | | YVFFQLELRR VVAGEGSGSV SLALHLQPLR |
| | | SAAGAAALAL TVDLPPASSE ARNSAFGFQG |
| | | RLLHLSAGQR LGVHLHTEAR ARHAWQLTQG |
| | | ATVLGLFRVT PEIPAGL |
| 66 | G₄S linker | GGGGS |
| | sequence | |
| 67 | (G₄S)₂ linker | GGGGGGGGS |
| | sequence | |
| 68 | (G₄S)₃ linker | GGGGSGGGGSGGGGS |
| | sequence | |
| 69 | (G₄S)₄ linker | GGGGSGGGGSGGGGSGGGGS |
| | sequence | |
| 70 | dimeric hu 4-1BBL | REGPELSPDDPAGLLDLRQGMFAQLVAQNVLLIDGPLS |
| | (71-254) connected | WYSDPGLAGVSLTGGLSYKEDTKELVVAKAGVYYVFF |
| | by (G₄S)₂ linker | QLELRRVVAGEGSGSVSLALHLQPLRSAAGAAALALTV |
| | | DLPPASSEARNSAFGFQGRLLHLSAGQRLGVHLHTEAR |
| | | ARHAWQLTQGATVLGLFRVTPEIPAGLPSPRSEGGGGSG |
| | | GGGSREGPELSPDDPAGLLDLRQGMFAQLVAQNVLLID |
| | | GPLSWYSDPGLAGVSLTGGLSYKEDTKELVVAKAGVY |
| | | YVFFQLELRRVVAGEGSGSVSLALHLQPLRSAAGAAAL |
| | | ALTVDLPPASSEARNSAFGFQGRLLHLSAGQRLGVHLHT |
| | | EARARHAWQLTQGATVLGLFRVTPEIPAGLPSPRSE |
| 71 | dimeric hu 4-1BBL | LDLRQGMFAQLVAQNVLLIDGPLSWYSDPGLAGVSLTG |
| | (85-254) connected | GLSYKEDTKELVVAKAGVYYVFFQLELRRVVAGEGSGS |
| | by (G₄S)₂ linker | VSLALHLQPLRSAAGAAALALTVDLPPASSEARNSAFGF |
| | | QGRLLHLSAGQRLGVHLHTEARARHAWQLTQGATVLG |
| | | LFRVTPEIPAGLPSPRSEGGGGSGGGGSLDLRQGMFAQL |
| | | VAQNVLLIDGPLSWYSDPGLAGVSLTGGLSYKEDTKEL |
| | | VVAKAGVYYVFFQLELRRVVAGEGSGSVSLALHLQPLR |
| | | SAAGAAALALTVDLPPASSEARNSAFGFQGRLLHLSAG |
| | | QRLGVHLHTEARARHAWQLTQGATVLGLFRVTPEIPAG |
| | | LPSPRSE |
| 72 | dimeric hu 4-1BBL | DPAGLLDLRQGMFAQLVAQNVLLIDGPLSWYSDPGLAG |
| | (80-254) connected | VSLTGGLSYKEDTKELVVAKAGVYYVFFQLELRRVVAG |
| | by (G₄S)₂ linker | EGSGSVSLALHLQPLRSAAGAAALALTVDLPPASSEARN |
| | | SAFGFQGRLLHLSAGQRLGVHLHTEARARHAWQLTQG |
| | | ATVLGLFRVTPEIPAGLPSPRSEGGGGSGGGGSDPAGLL |
| | | DLRQGMFAQLVAQNVLLIDGPLSWYSDPGLAGVSLTGG |
| | | LSYKEDTKELVVAKAGVYYVFFQLELRRVVAGEGSGSV |
| | | SLALHLQPLRSAAGAAALALTVDLPPASSEARNSAFGFQ |
| | | GRLLHLSAGQRLGVHLHTEARARHAWQLTQGATVLGL |
| | | FRVTPEIPAGLPSPRSE |
| 73 | dimeric hu 4-1BBL | PWAVSGARASPGSAASPRLREGPELSPDDPAGLLDLRQG |
| | (52-254) connected | MFAQLVAQNVLLIDGPLSWYSDPGLAGVSLTGGLSYKE |
| | by (G₄S)₂ linker | DTKELVVAKAGVYYVFFQLELRRVVAGEGSGSVSLALH |
| | | LQPLRSAAGAAALALTVDLPPASSEARNSAFGFQGRLLH |
| | | LSAGQRLGVHLHTEARARHAWQLTQGATVLGLFRVTP |
| | | EIPAGLPSPRSEGGGGSGGGGSPWAVSGARASPGSAASP |
| | | RLREGPELSPDDPAGLLDLRQGMFAQLVAQNVLLIDGPL |
| | | SWYSDPGLAGVSLTGGLSYKEDTKELVVAKAGVYYVF |
| | | FQLELRRVVAGEGSGSVSLALHLQPLRSAAGAAALALT |
| | | VDLPPASSEARNSAFGFQGRLLHLSAGQRLGVHLHTEA |
| | | RARHAWQLTQGATVLGLFRVTPEIPAGLPSPRSE |
| 74 | dimeric hu 4-1BBL | REGPELSPDDPAGLLDLRQGMFAQLVAQNVLLIDGPLS |
| | (71-248) connected | WYSDPGLAGVSLTGGLSYKEDTKELVVAKAGVYYVFF |
| | by (G₄S)₂ linker | QLELRRVVAGEGSGSVSLALHLQPLRSAAGAAALALTV |
| | | DLPPASSEARNSAFGFQGRLLHLSAGQRLGVHLHTEAR |
| | | ARHAWQLTQGATVLGLFRVTPEIPAGLGGGGSGGGGSR |
| | | EGPELSPDDPAGLLDLRQGMFAQLVAQNVLLIDGPLSW |
| | | YSDPGLAGVSLTGGLSYKEDTKELVVAKAGVYYVFFQL |
| | | ELRRVVAGEGSGSVSLALHLQPLRSAAGAAALALTVDL |
| | | PPASSEARNSAFGFQGRLLHLSAGQRLGVHLHTEARAR |
| | | HAWQLTQGATVLGLFRVTPEIPAGL |
| 75 | dimeric hu 4-1BBL | LDLRQGMFAQLVAQNVLLIDGPLSWYSDPGLAGVSLTG |
| | (85-248) connected | GLSYKEDTKELVVAKAGVYYVFFQLELRRVVAGEGSGS |
| | by (G₄S)₂ linker | VSLALHLQPLRSAAGAAALALTVDLPPASSEARNSAFGF |
| | | QGRLLHLSAGQRLGVHLHTEARARHAWQLTQGATVLG |
| | | LFRVTPEIPAGLGGGGSGGGGSLDLRQGMFAQLVAQNV |
| | | LLIDGPLSWYSDPGLAGVSLTGGLSYKEDTKELVVAKA |
| | | GVYYVFFQLELRRVVAGEGSGSVSLALHLQPLRSAAGA |
| | | AALALTVDLPPASSEARNSAFGFQGRLLHLSAGQRLGV |
| | | HLHTEARARHAWQLTQGATVLGLFRVTPEIPAGL |
| 76 | dimeric hu 4-1BBL | DPAGLLDLRQGMFAQLVAQNVLLIDGPLSWYSDPGLAG |
| | (80-248) connected | VSLTGGLSYKEDTKELVVAKAGVYYVFFQLELRRVVAG |
| | by (G₄S)₂ linker | EGSGSVSLALHLQPLRSAAGAAALALTVDLPPASSEARN |
| | | SAFGFQGRLLHLSAGQRLGVHLHTEARARHAWQLTQG |
| | | ATVLGLFRVTPEIPAGLGGGGSGGGGSDPAGLLDLRQG |
| | | MFAQLVAQNVLLIDGPLSWYSDPGLAGVSLTGGLSYKE |
| | | DTKELVVAKAGVYYVFFQLELRRVVAGEGSGSVSLALH |
| | | LQPLRSAAGAAALALTVDLPPASSEARNSAFGFQGRLLH |
| | | LSAGQRLGVHLHTEARARHAWQLTQGATVLGLFRVTP |
| | | EIPAGL |
| 77 | dimeric hu 4-1BBL | PWAVSGARASPGSAASPRLREGPELSPDDPAGLLDLRQG |
| | (52-248) connected | MFAQLVAQNVLLIDGPLSWYSDPGLAGVSLTGGLSYKE |
| | by (G₄S)₂ linker | DTKELVVAKAGVYYVFFQLELRRVVAGEGSGSVSLALH |
| | | LQPLRSAAGAAALALTVDLPPASSEARNSAFGFQGRLLH |
| | | LSAGQRLGVHLHTEARARHAWQLTQGATVLGLFRVTP |
| | | EIPAGLGGGGSGGGGSPWAVSGARASPGSAASPRLREGP |
| | | ELSPDDPAGLLDLRQGMFAQLVAQNVLLIDGPLSWYSD |
| | | PGLAGVSLTGGLSYKEDTKELVVAKAGVYYVFFQLELR |
| | | RVVAGEGSGSVSLALHLQPLRSAAGAAALALTVDLPPA |
| | | SSEARNSAFGFQGRLLHLSAGQRLGVHLHTEARARHAW |
| | | QLTQGATVLGLFRVTPEIPAGL |
| 78 | hu 4-1BBL (71- | REGPELSPDDPAGLLDLRQGMFAQLVAQNVLLIDGPLS |
| | 248)-(G₄S)₂-hu 4- | WYSDPGLAGVSLTGGLSYKEDTKELVVAKAGVYYVFF |
| | 1BBL (71-248)- | QLELRRVVAGEGSGSVSLALHLQPLRSAAGAAALALTV |
| | CL(RK) | DLPPASSEARNSAFGFQGRLLHLSAGQRLGVHLHTEAR |
| | | ARHAWQLTQGATVLGLFRVTPEIPAGLGGGGSGGGGSR |
| | | EGPELSPDDPAGLLDLRQGMFAQLVAQNVLLIDGPLSW |
| | | YSDPGLAGVSLTGGLSYKEDTKELVVAKAGVYYVFFQL |
| | | ELRRVVAGEGSGSVSLALHLQPLRSAAGAAALALTVDL |
| | | PPASSEARNSAFGFQGRLLHLSAGQRLGVHLHTEARAR |
| | | HAWQLTQGATVLGLFRVTPEIPAGLGGGGSGGGGSRTV |
| | | AAPSVFIFPPSDRKLKSGTASVVCLLNNFYPREAKVQWK |
| | | VDNALQSGNSQESVTEQDSKDSTYSLSSTLTLSKADYEK |
| | | HKVYACEVTHQGLSSPVTKSFNRGEC |
| 79 | hu 4-1BBL (71- | REGPELSPDDPAGLLDLRQGMFAQLVAQNVLLIDGPLS |
| | 248)-(G₄S)₂-hu 4- | WYSDPGLAGVSLTGGLSYKEDTKELVVAKAGVYYVFF |
| | 1BBL (71-248)- | QLELRRVVAGEGSGSVSLALHLQPLRSAAGAAALALTV |
| | CL(RK)-CH2-CH3 | DLPPASSEARNSAFGFQGRLLHLSAGQRLGVHLHTEAR |
| | (knob, PGLALA) | ARHAWQLTQGATVLGLFRVTPEIPAGLGGGGSGGGGSR |
| | | EGPELSPDDPAGLLDLRQGMFAQLVAQNVLLIDGPLSW |
| | | YSDPGLAGVSLTGGLSYKEDTKELVVAKAGVYYVFFQL |
| | | ELRRVVAGEGSGSVSLALHLQPLRSAAGAAALALTVDL |
| | | PPASSEARNSAFGFQGRLLHLSAGQRLGVHLHTEARAR |
| | | HAWQLTQGATVLGLFRVTPEIPAGLGGGGSGGGGSRTV |
| | | AAPSVFIFPPSDRKLKSGTASVVCLLNNFYPREAKVQWK |
| | | VDNALQSGNSQESVTEQDSKDSTYSLSSTLTLSKADYEK |
| | | HKVYACEVTHQGLSSPVTKSFNRGECDKTHTCPPCPAPE |
| | | AAGGPSVFLFPPKPKDTLMISRTPEVTCVVVDVSHEDPE |
| | | VKFNWYVDGVEVHNAKTKPREEQYNSTYRVVSVLTVL |
| | | HQDWLNGKEYKCKVSNKALGAPIEKTISKAKGQPREPQ |
| | | VYTLPPCRDELTKNQVSLWCLVKGFYPSDIAVEWESNG |
| | | QPENNYKTTPPVLDSDGSFFLYSKLTVDKSRWQQGNVF |
| | | SCSVMHEALHNHYTQKSLSLSP |
| 80 | hu 4-1BBL (71- | REGPELSPDDPAGLLDLRQGMFAQLVAQNVLLIDGPLS |
| | 248)-CH1(EE) | WYSDPGLAGVSLTGGLSYKEDTKELVVAKAGVYYVFF |
| | | QLELRRVVAGEGSGSVSLALHLQPLRSAAGAAALALTV |
| | | DLPPASSEARNSAFGFQGRLLHLSAGQRLGVHLHTEAR |
| | | ARHAWQLTQGATVLGLFRVTPEIPAGLGGGGSGGGGSA |
| | | STKGPSVFPLAPSSKSTSGGTAALGCLVEDYFPEPVTVS |
| | | WNSGALTSGVHTFPAVLQSSGLYSLSSVVTVPSSSLGTQ |
| | | TYICNVNHKPSNTKVDEKVEPKSC |
| 81 | CH1(EE) | ASTKGPSVFPLAPSSKSTSGGTAALGCLVEDYFPEPVTVS |
| | | WNSGALTSGVHTFPAVLQSSGLYSLSSVVTVPSSSLGTQ |
| | | TYICNVNHKPSNTKVDEKVEPKSC |
| 82 | CL(RK) | RTVAAPSVFIFPPSDRKLKSGTASVVCLLNNFYPREAKV |
| | | QWKVDNALQSGNSQESVTEQDSKDSTYSLSSTLTLSKA |
| | | DYEKHKVYACEVTHQGLSSPVTKSFNRGEC |

The present disclosure includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

As used herein, an amino acid sequence, or a region of a polypeptide which ‘corresponds’ to a specified reference amino acid sequence or region of a polypeptide has at least 60%, e.g. one of at least ≥65%, ≥70%, ≥75%, ≥80%, ≥85%, ≥90%, ≥91%, ≥92%, ≥93%, ≥94%, ≥95%, ≥96%, ≥97%, ≥98%, ≥99% or 100% sequence identity to the amino acid sequence of the amino acid sequence/polypeptide/region. An amino acid sequence/region/position of a polypeptide/amino acid sequence which ‘corresponds’ to a specified reference amino acid sequence/region/position of a polypeptide/amino acid sequence can be identified by sequence alignment of the subject sequence to the reference sequence, e.g. using sequence alignment software such as ClustalOmega (Söding, J. 2005, Bioinformatics 21, 951-960).

Where an amino acid (other than glycine) is referred to herein, both L and D enantiomers of the relevant amino acid are expressly contemplated. In some embodiments, except where specifically indicated otherwise, reference to an amino acid herein refers particularly to the L enantiomer, this being the form of the amino acid as it occurs in nature.

It must be noted that, as used in the specification and the appended claims, the singular forms ‘a’, ‘an’, and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about’, it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

Values may be expressed herein as ‘about’ a particular value. Similarly, ranges may be expressed herein as from ‘about’ a particular value, and/or to ‘about’ another particular value. The term ‘about’ in relation to a numerical value is optional, and means for example +/−10%. By way of illustration, reference e.g. to ‘about 10%’ is to be construed as 9% to 11%. In instances herein where ‘about’ is recited, the value it precedes is also specifically contemplated. By way of illustration, reference e.g. to ‘about 10%’ also specifically contemplates 10%.

### BRIEF DESCRIPTION OF THE FIGURES

Embodiments and experiments illustrating the principles of the present disclosure will now be discussed with reference to the accompanying figures.

FIGS. 1A and 1B. Schematic representations of the structures of (1A) the KiH molecule, and (1B) Hole-hole molecule, ½ Knob molecule and ½ Hole molecule products of cell cultures performed as described in Example 1.

FIG. 2. Graph showing the cumulative molar levels of cysteine in the absence of metabolically active cellular consumers over a period of 14 days.

FIGS. 3A, 3B, 3C, 3D, 3E and 3F. Bar charts summarising the titers of products, and the proportions of different products, purified by protein A affinity chromatography from the cell culture supernatants performed as described in Example 1. Each bar corresponds to a single experiment; for each experimental condition, n=2. (3A) shows the titer of all products purified by protein A affinity chromatography from the cell culture supernatants. (3B) shows the effective product titer obtained from the different cultures, which is calculated as the titer of all products multiplied by the fraction of the products which are the main product (i.e. the KiH molecule). (3C) shows the fraction of products which are the main product (i.e. the KiH molecule), among the products obtained from the different cultures. (3D) shows the fraction of products which are the Hole-hole molecule among the products obtained from the different cultures. (3E) shows the fraction of products which are the ½ Hole molecule among the products obtained from the different cultures. (3F) shows the ratio of the Hole-hole molecule to the main product (i.e. the KiH molecule) among the products obtained from the different cultures, which is calculated as the fraction of the products which are the Hole-hole molecule (%) divided by the fraction of the products which are the main product (%) (i.e. the KiH molecule).

FIG. 4. Histogram summarising the relative proportions of the different molecules detected by non-reducing CE-SDS analysis of the products of cell culture purified by protein A affinity chromatography. The titers of protein A-purified products obtained by culture according to the different methods are also shown.

FIG. 5. Schematic representations of six different KiH molecules (products A to F) used to test the influence of different Cysteine levels in base medium and feed to analyse the impact on the KiH main product level.

FIG. 6. Design of Experiments (DoE) Model design examples which are routinely used for optimisation of multidimensional parameter spaces.

FIG. 7A and FIG. 7B. Theoretical, cumulative Cysteine concentration trajectories in a 14-day fed-batch bioreactor without cellular consumers but with a representative sampling event is calculated and plotted to represent the overall molar Cysteine system ‘intake’ or ‘dilution’ in different test cases.

FIG. 8. Design of Experiments (DoE) model for hole-hole and main product observed vs predicted levels and influence residues for Medium A.

FIG. 9. Design of Experiments (DoE) model for hole-hole and main product observed vs predicted levels and influence residues for commercial medium.

FIG. 10. Global Design of Experiments (DoE) model for hole-hole and main product observed vs predicted levels for combined media types.

FIGS. 11A and 11B. Design of Experiments (DoE) model prediction profiler plots for hole-hole and main product levels in (11A) medium A or (11B) commercial medium.

FIG. 12. Delta improved main product level of KiH products A to F in low vs high cysteine cell culture conditions.

FIG. 13. Bar charts showing the improved Hole-hole (HH) and main product levels of KiH products in Cysteine base medium vs Cysteine feed by a comparison between an osmolality of 300 vs 370 mOsmol/kg.

FIG. 14. Bar charts showing the delta improved main product and reduced Hole-hole (HH) level of KiH product A in Cysteine base medium vs Cysteine feed by a comparison between an osmolality of 300 vs 370 mOsmol/kg.

### EXAMPLES

### Example 1

The inventors investigated the production of a bispecific molecules comprising a Knobs-into-Holes (KiH) Fc moiety, a target antigen-binding Fab moiety and a 4-1BB-binding moiety formed of three copies of the amino acid sequence of the extracellular domain of 4-1BBL.

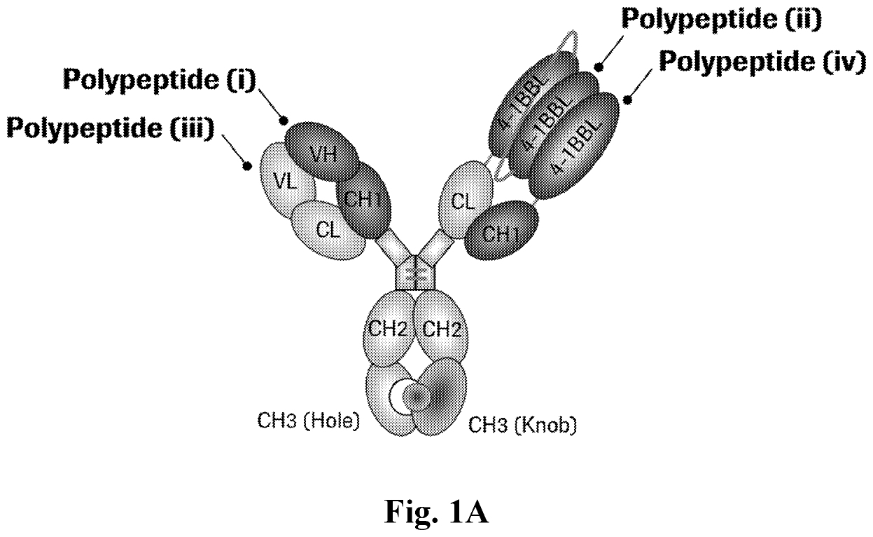

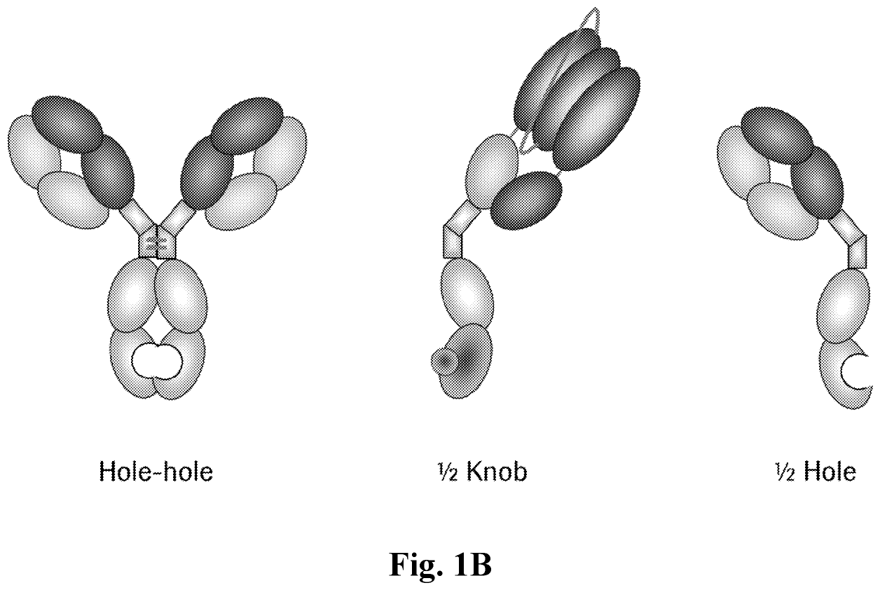

The desired product is represented schematically in FIG. 1A (‘KiH’ Molecule), and is formed from:
(i) a polypeptide comprising, from N-term to C-term:
   (a) the VH region of an antibody that binds to a target antigen;
   (b) a CH1 region;
   (c) a CH1-CH2 hinge region;
   (d) a CH2 region; and
   (e) a CH3 region comprising a hole modification;
(ii) a polypeptide comprising, from N-term to C-term:
   (a) two copies of the extracellular domain of 4-1BBL joined by linker sequences;
   (b) a CL region;
   (c) a CL-CH2 hinge region;
   (e) a CH2 region; and
   (f) a CH3 region comprising a knob modification;
(iii) a polypeptide comprising, from N-term to C-term:
   (a) the VL region of the antibody of (i)(a); and
   (b) a CL region; and
(iv) a polypeptide comprising, from N-term to C-term:
   (a) the extracellular domain of 4-1BBL; and
   (b) a CH1 region.

The molecule is a heteromeric polypeptide complex, in which polypeptides (i) and (ii) associate with one another via interaction between their respective CH2-CH3 regions, and in which polypeptides (i) and (iii) associate with one another via interaction between the VL-CL region of polypeptide (iii) with the VH—CH1 region of polypeptide (i), and in which polypeptides (ii) and (iv) associate with one another via interaction between the CH1-4-1BBL region of polypeptide (iv) with the CL-4-1BBL region of polypeptide (ii).

FIG. 1B shows exemplary undesired products of cells comprising expression vectors for co-expressing polypeptides (i), (ii), (iii) and (iv).

The ‘Hole-hole’ molecule is a heteromeric polypeptide complex comprising a homodimer of monomers polypeptide (i), formed by interaction between their CH2-CH3 regions. The molecule further comprises two monomers of polypeptide (iii), each monomer associating with a polypeptide (i) monomer via interaction between the VL-CL region of polypeptide (iii) and the VH—CH1 region of polypeptide (i).

The ‘½ knob’ molecule is a heterodimeric polypeptide complex comprising a polypeptide (ii) monomer associated with a polypeptide (iv) monomer, via interaction between the CL-4-1BBL region of polypeptide (ii) and the CH1-4-1BBL region of polypeptide (iv).

The ‘½hole’ molecule is a heterodimeric polypeptide complex comprising a polypeptide (i) monomer associated with a polypeptide (iii) monomer, via interaction between the VL-CL region of polypeptide (iii) and the VH—CH1 region of polypeptide (i).

The inventors cultured CHO-K1 cells comprising expression vectors for co-expressing polypeptides (i), (ii), (iii) and (iv) under different cell culture conditions, using a fed-batch cell culture process.

Pre-culture for inoculation of the main cultures were performed in temperature-, CO₂— and humidity-controlled shake flask cultures and incubators from Mytron.

Fed-batch cell culture was performed in 2 L, fully-controlled bioreactors which are connected to a control unit to control the temperature, pH, dissolved oxygen (pO₂), aeration and agitation of the culture. Online process data (such as pO₂, temperature, pH, stirrer speed, gassing) were recorded with the fermentation control system. External pH and pCO₂ measurements were monitored using a blood gas analyzer system (pHOx, IUL instruments). Viable and total cell densities as well as viability were evaluated using a Cedex HiRes system (Roche). Concentrations of product, substrates, and metabolites in cell culture supernatants were determined utilizing a CedexBioHT (Roche) and a RRLC 1200 system (Agilent). Osmolality was determined using the osmometer system OSMOMAT® auto (Gonotec).

Cell cultures were performed using chemically-defined base media A or B. Cells seeded in base medium A were seeded at 10E5 cells/ml, and cells seeded in base medium B were seeded at 3.5E5 cells/mL. The chemically-defined base media A and B consist of different mixtures of an appropriate carbon source (e.g. glucose), amino acids and amino acid derivatives, vitamins and vitaminoids, polyamines, lipids and bulk salts.

During the 14 to 16 day fed-batch cell culture process, the temperature was maintained at 36.5° C., 30% pO₂, and pH 7.0.

For fed-batch processes, two different nutrient feeds (1 and 2) were provided, as summarised below.

| Process | Base Medium | Feed 1 | Feed 2 |
|---|---|---|---|
| Process A | Medium A | Feed 1a | Feed 2a |
| | | (carbon source, | (amino acid) |
| | | amino acids, | Bolus feed with |
| | | vitamins, | 0.225% (v/vₛₜₐᵣₜ) |
| | | vitaminoids, | of 1M solution |
| | | bulk salts) | at day 3 and day 7 |
| | | Continuous feed | |
| | | with 2.73% | |
| | | (v/vₛₜₐᵣₜ) from | |
| | | day 3 to end | |
| Process B | Medium B | Feed 1b | Feed 2b |
| | | (amino acids, | (carbon source, |
| | | polyamines, | amino acids, |
| | | bulk salts) | vitamins, vitaminoids, |
| | | Continuous feed | bulk salts) |
| | | with 2% | Continuous feed |
| | | (v/v_{start Bioreactor}) | with 2% |
| | | from day 3 to end | (v/v_{start Bioreactor}) from |
| | | | day 6 to end |

The concentration of cysteine in the feeds was controlled in order to achieve the final cysteine concentrations during the cultivations.

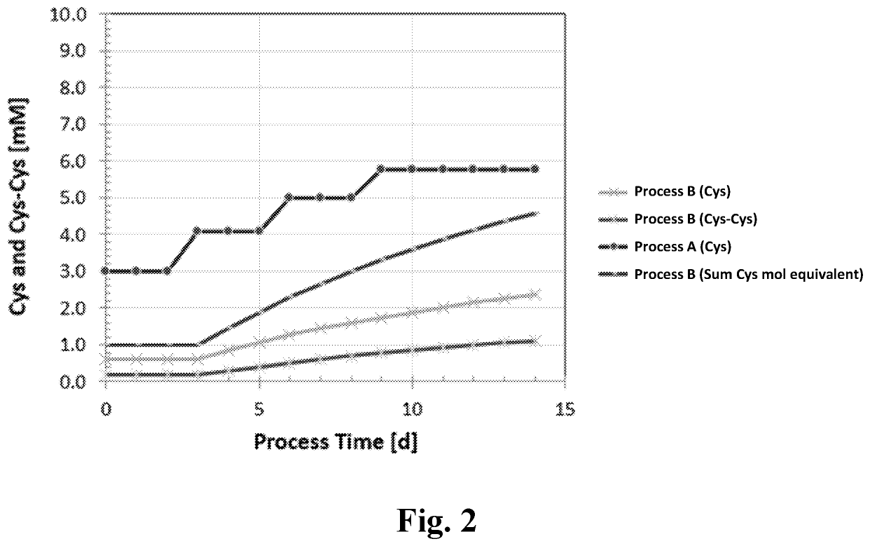

Cysteine levels accumulate in the absence of a metabolically active cellular consumer, as exemplified by FIG. 2. If cellular consumers are present, the actual measurable cysteine levels will be lower. The delta between the theoretical cumulative cysteine and the actual measured cysteine concentration when cellular consumers are present is the amount of cysteine the active biomass consumes. The specific cysteine consumption rate, Schf, can be calculated ifthe biomass and time of consumption are considered.

For ‘osmolality shift’ experiments, the osmolality of the cell culture shifted from 300 mOsmol/kg to 400 mOsmol/kg using a NaCl solution, either by a bolus at day 7 (Osmo shift at day 7), or by a ramping profile from day 3 to day 7 (Osmo Shift from day 3 to 7). The shift strategies were designed to be effective after the growth phase and before the production phase, which typically occurs from day 8 of cultivation.

The different cell culture conditions evaluated are summarised in the table below:

| | | | Base | | | | Osmo | | |
|---|---|---|---|---|---|---|---|---|---|
| | | | Medium | Base | Feed | Osmo | Shift | Har- | Har- |
| | | | Osmolality | Medium | Medium | Shift | from | vest | vest |
| | Repli- | Base | [mOsmol/ | Cys | Cys | at | day 3 to | day | day |
| Run | cate | Medium | kg] | [mM] | [mM] | day 7 | day 7 | 14 | 17 |
| 1 | 1 | A | 370 | 6 | 15 | No | Yes | Yes | No |
| 2 | 2 | A | 370 | 6 | 15 | No | Yes | Yes | No |
| 3 | 1 | A | 370 | 6 | 15 | Yes | No | Yes | No |
| 4 | 2 | A | 370 | 6 | 15 | Yes | No | Yes | No |
| 5 | 1 | A | 300 | 3 | 0 | No | No | Yes | No |
| 6 | 2 | A | 300 | 3 | 0 | No | No | Yes | No |
| 7 | 1 | A | 300 | 3 | 15 | No | No | Yes | No |
| 8 | 2 | A | 300 | 3 | 15 | No | No | Yes | No |
| 9 | 1 | A | 300 | 6 | 0 | No | No | Yes | No |
| 10 | 2 | A | 300 | 6 | 0 | No | No | Yes | No |
| 11 | 1 | B | 370 | 1 | 12 | No | No | Yes | No |
| 12 | 2 | B | 370 | 1 | 12 | No | No | Yes | No |
| 13 | 1 | B | 370 | 1 | 12 | No | No | No | Yes |
| 14 | 2 | B | 370 | 1 | 12 | No | No | No | Yes |
| 15 | 1 | A | 370 | 6 | 15 | No | No | Yes | No |
| 16 | 2 | A | 370 | 6 | 15 | No | No | Yes | No |
| 17 | 1 | A | 370 | 3 | 0 | No | No | Yes | No |
| 18 | 2 | A | 370 | 3 | 0 | No | No | Yes | No |
| 19 | 1 | A | 370 | 3 | 15 | No | No | Yes | No |
| 20 | 2 | A | 370 | 3 | 15 | No | No | Yes | No |

The cell culture supernatant was harvested at 14-16 days after start of fermentation by centrifugation (10 min, 1000 rpm followed by 10 min, 4000 rpm) and cleared by 0.22 μm filtration.

To determine the yield and proportions of the products of the cell culture, the product concentration in the cell-free supernatants was measured using CedexBioHT (Roche). Cell-free supernatants were micropurified by protein A. Briefly, Protein A affinity chromatography was used for purification of the antibody-derived target protein, as the Fc moiety comprised in the target molecule is known to have a high affinity for Protein A. Protein A purification of small volumes of the cell-free supernatant (150 μl-3 ml) was performed using PureSpeed tips (Mettler Toledo, Columbus, OH) or OPUS RoboColumns (Repligen, Waltham, MA).

The protein A-purified molecules were subsequently analyzed by non-reducing CE-SDS for main and side-product proofing (as percentage abundance). The small delta to 100% represent a fraction of unknown peaks which could not be annotated.

The CE-SDS was performed under non-reducing conditions using a LabChipGXII (Perkin Elmer) system, in accordance with the manufacturer's instructions. The desired KiH molecule product has a size of approximately 189 kDa, the Hole-hole molecule has a size of approximately 165 kDa, the ½ Knob molecule has a size of approximately 143 kDa, and the ½ Hole molecule has a size of approximately 95 kDa. Unknown side products having sizes of approximately 130 kDa and 125 kDa were also detected.

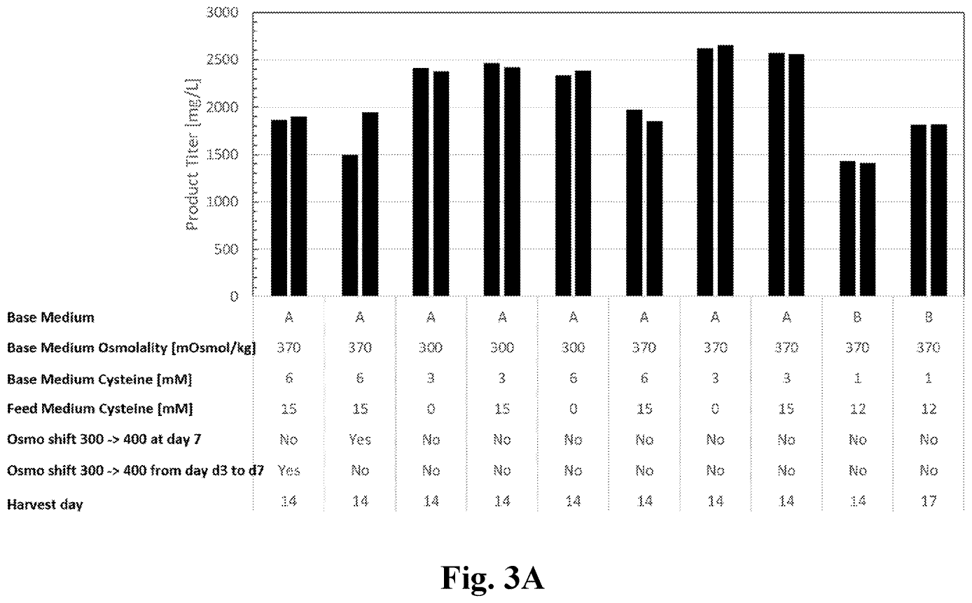

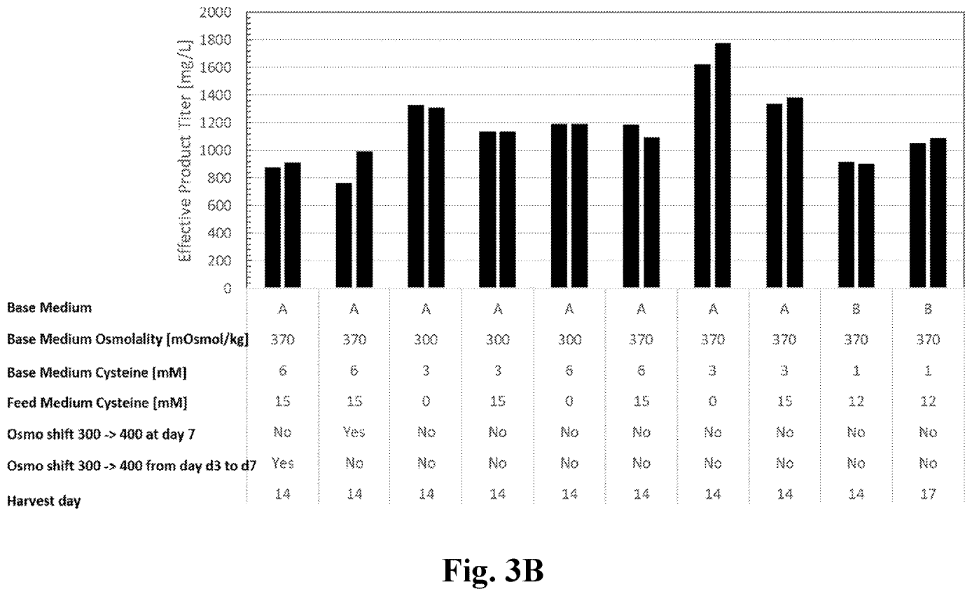

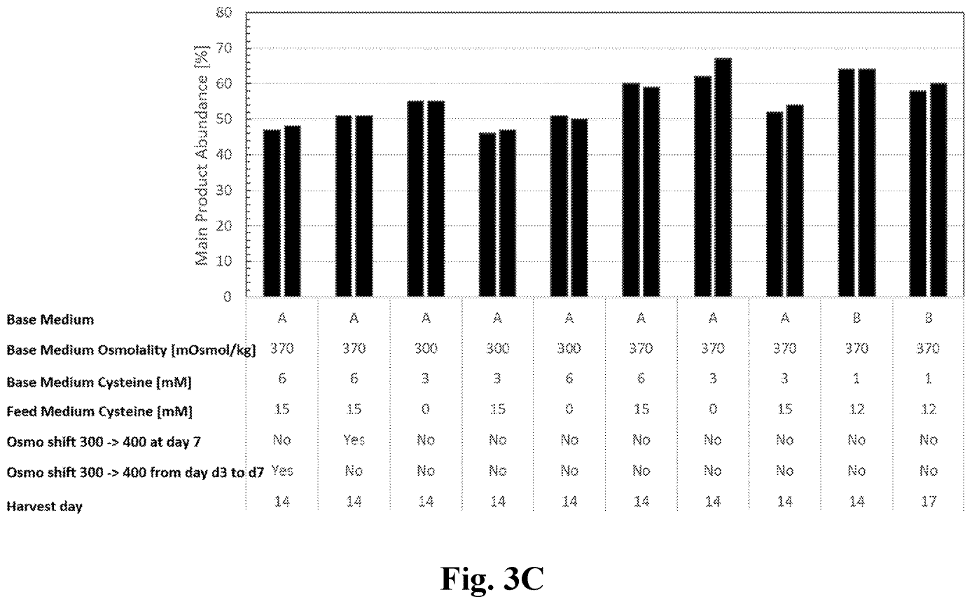

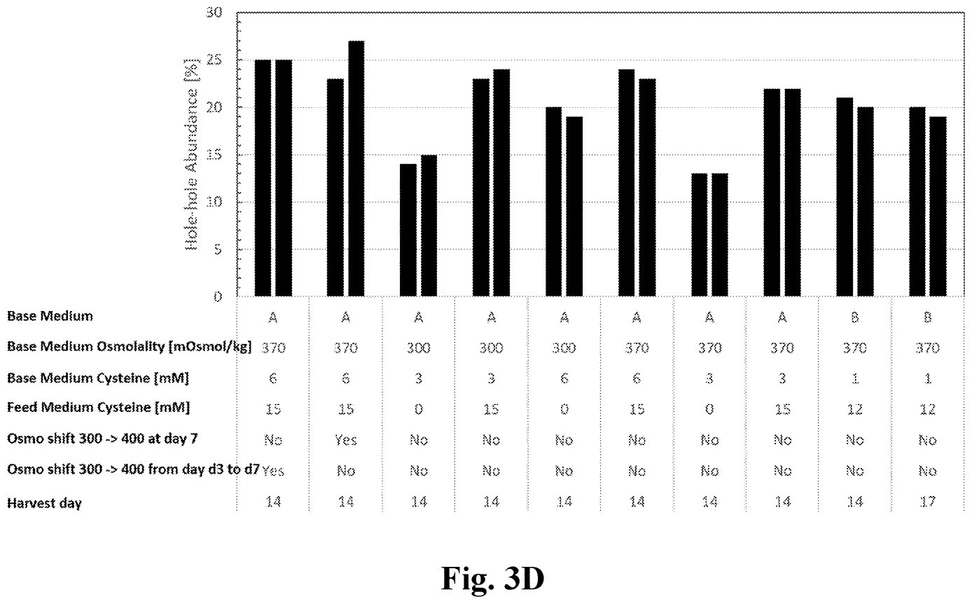

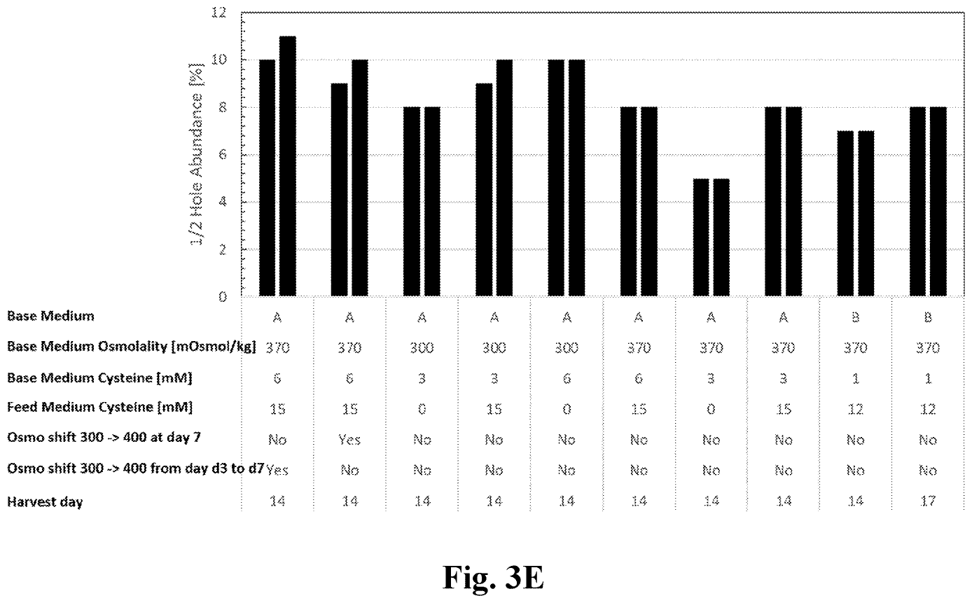

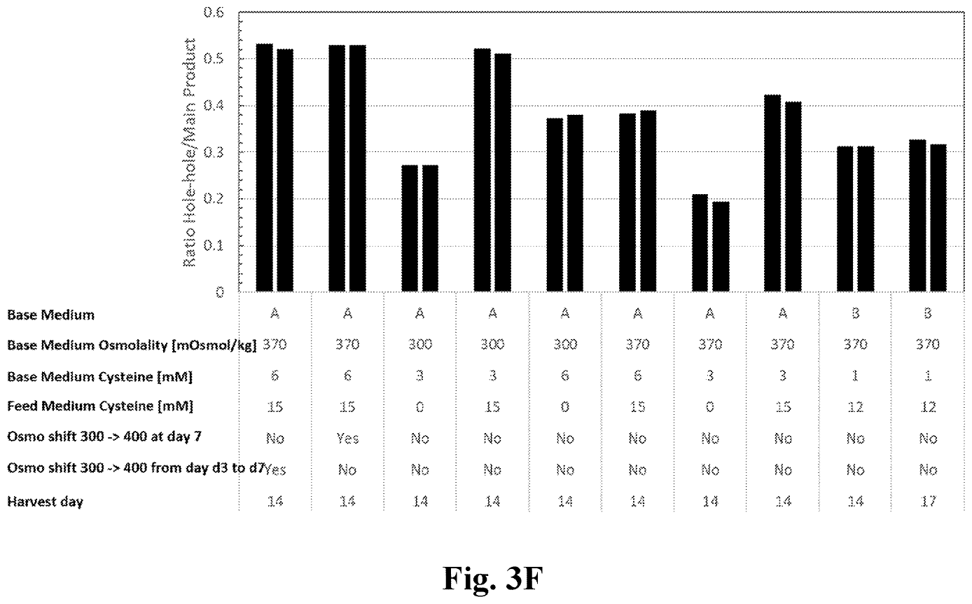

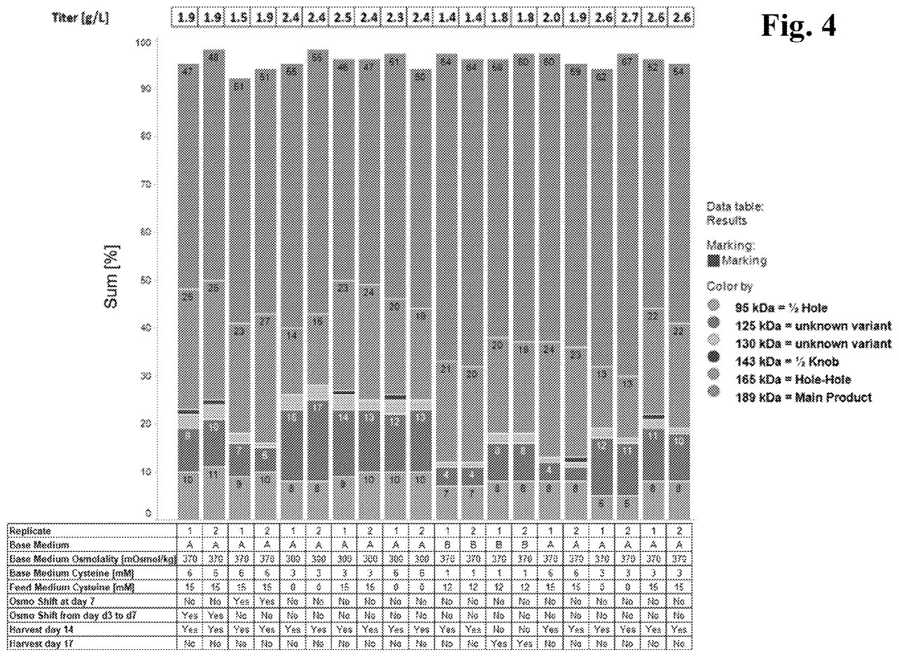

The titers of products purified from the cell culture supernatants by protein A affinity chromatography and the relative proportions of the different molecules detected are shown in FIGS. 3A to 3F, and summarised in FIG. 4.

### Example 2

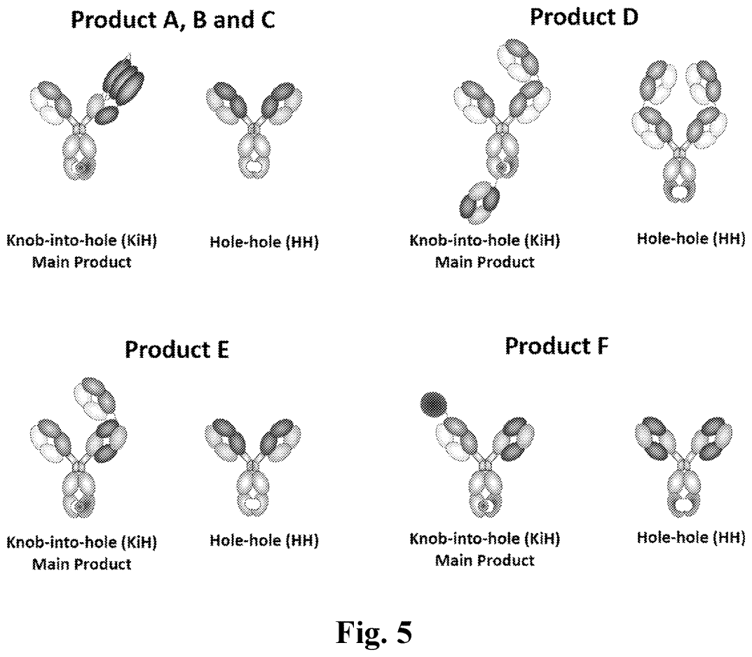

To elucidate the impact of cysteine concentration in cell culture base medium and feed, the base medium osmolality and cell culture pH setpoint on KiH main product level in harvested cell culture fluid, a comprehensive DoE (Design of Experiments)-based experiment was designed. In addition, using a commercially available mammalian cell culture medium, the transferability of cysteine relevance for KiH content formation was tested. Furthermore, we used six different KiH molecules (FIG. 5) in combination with different high and low cysteine cell culture nutrient strategies and analyzed the respective impact on the KiH main product levels. In a first experiment series, experiment 1-24, different cysteine levels in the cell culture base medium and in the nutrient feed were tested on Product A, in both a Process C and commercial medium background. In a subsequent experimental series, experiments 25-48, six different KiH molecules (FIG. 5) were used for testing the influence of different cysteine levels in base medium and feed and for evaluating cell culture pH and base medium start osmolality at inoculation. In summary, 48 single experiments were performed using an ambr15 fermentation robotic device (Sartorius Stedim, Gottingen, Germany).

| Process | Base Medium | Feed 1 | Feed 2 |
|---|---|---|---|
| Process C | Medium A | Feed 1c | Feed 2c |
| | Cysteine levels | (carbon source, | (amino acids) |
| | according Table 2 | amino acids, | Bolus feed with |
| | ‘Base Medium | vitamins, | 0.68% (v/v_{actual}) |
| | Cys’ | vitaminoids, | at day 3, 6, 9, and |
| | | bulk salts) | 12 |
| | | Cysteine levels | |
| | | according Table | |
| | | 2 ‘Feed Medium | |
| | | Cys’ | |
| | | Bolus feed with | |
| | | 7.5% at day 3, 6, | |
| | | 9, and 12 | |
| | | (v/v_{actual}) | |
| Process Com | DMEM (Thermo | Feed 1c | Feed 2c |
| (=commercial) | Fisher Scientific, | (carbon source, | (amino acids) |
| | catalogue No. | amino acids, | Bolus feed with |
| | 21013024) | vitamins, | 0.68% (v/v_{actual}) |
| | Cysteine levels | vitaminoids, | at day 3, 6, 9, and |
| | according Table 2 | bulk salts) | 12 |
| | ‘Base Medium | Cysteine levels | |
| | Cys’ | according Table | |
| | | 2 ’Feed Medium | |
| | | Cys’ | |
| | | Bolus feed with | |
| | | 7.5% at day 3, 6, | |
| | | 9, and 12 | |
| | | (v/v_{actual}) | |

### DoE Experiment

Statistical experiment planning by Design of Experiments (DoE) is a powerful and well-known technique especially for biological and chemical reaction optimization intentions where many factors interdependently influence the result. For example, DoE was successfully used for optimization of cell culture media (Zhang et al. 363-78), fermentation processes (Fu et al. 1095-105), protein purification processes (Pezzini et al. 8197-208) and cell culture conditions (Chen et al. 1211-21).

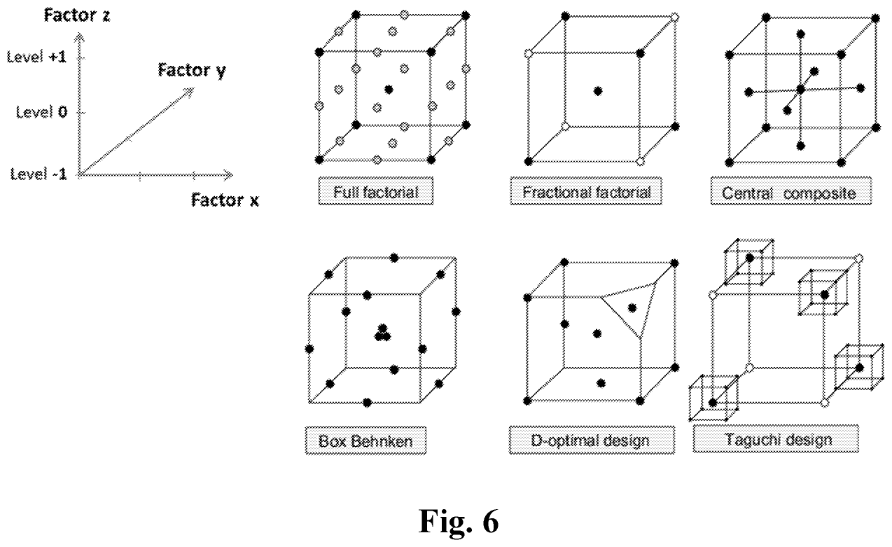

Different DoE approaches can be applied to the present process depending on the desired outcome (FIG. 6). For example, the full factorial design is well suited for situations, where 1) only few factors are considered, 2) mainly linear effects are of interest and 3) a large number of experiments is feasible.

The DoE approach in this study was planned and analyzed using statistical software tool JMP (SAS Institute GmbH, Böblingen, Germany). The DoE results and importance of tested parameters were shown by ‘actual vs. predicted’ plot feature of the JMP software. For this DoE study, a ‘good model quality’ is defined by a p-value<0.05, a RMSE-value<10% of the median of the observed/analysed parameter, and an r2>0.8.

The ranges of the tested parameters and variables in 48 single experiments embedded in a DoE approach are listed in Table 1. For the fed-batch processes, two different media were used; medium A and the commercially available medium DMEM (Thermo Fisher Scientific, Waltham, MA USA; catalogue No 21013024; named ‘Com’=commercial medium in Table 1). Feeding was realized by two different nutrient feeds (feed 1 and feed 2), as summarised below.

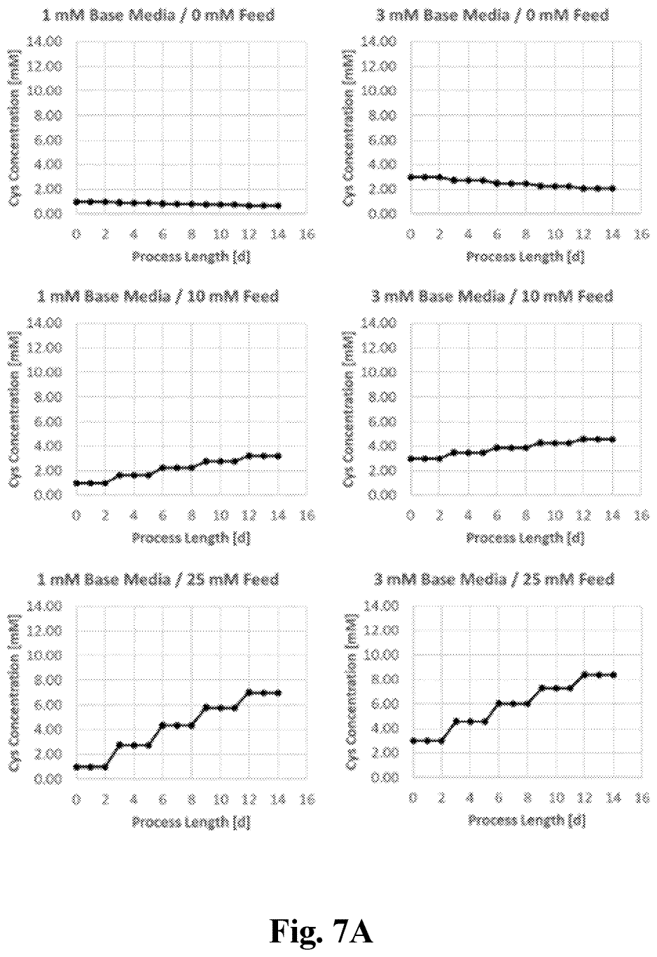

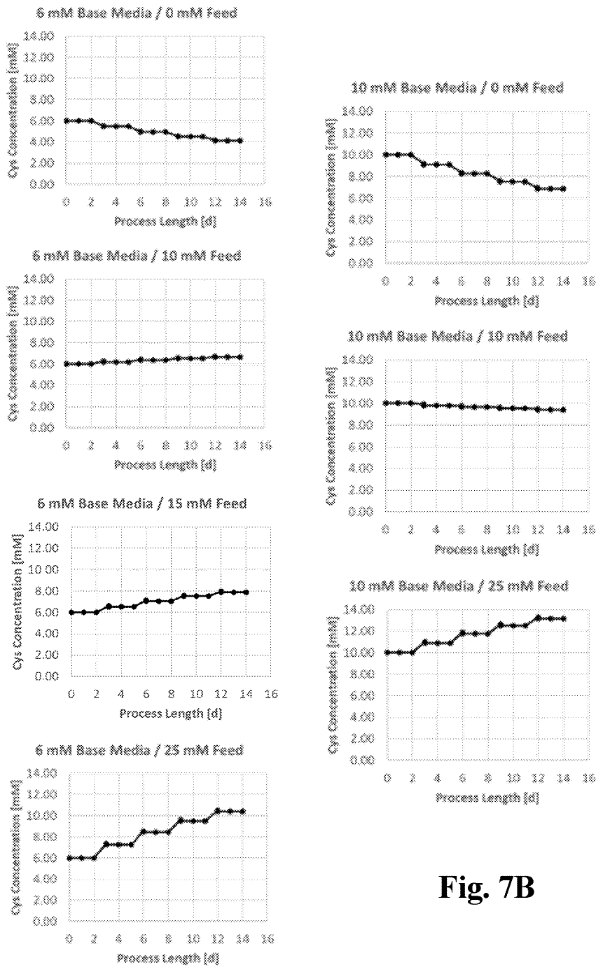

The concentration of cysteine in the feeds was controlled in order to achieve the final cysteine concentrations during the cultivations. The theoretical cysteine concentration trajectories in the fed-batch experiments without ‘consumer’ (such as ‘cells’, chemical reactions, precipitations, etc.), but with a representative sampling event is calculated and plotted in FIGS. 7A and 7B to represent the overall molar Cysteine system ‘intake’ or ‘dilution’ (increase/decrease of global Cysteine concentration by mass balancing of base medium and nutrient feed) in the different test cases.

TABLE 1
Conditions for the DoE and parameter test fermentation runs
(Abbr. in Base Medium: Com = Commercial)

| | | | Base | | | |
|---|---|---|---|---|---|---|
| | | | Medium | Feed Medium | Base Medium | |
| | Base | | Cys | Cys | Osmolality | Process pH |
| Run | Medium | Product | [mM] | [mM] | [mOsmol/kg] | Setpoint |
| 1 | A | Product A | 1 | 0 | 370 | 7.2 |
| 2 | A | Product A | 3 | 0 | 370 | 7.2 |
| 3 | A | Product A | 6 | 0 | 370 | 7.2 |
| 4 | A | Product A | 10 | 0 | 370 | 7.2 |
| 5 | A | Product A | 1 | 10 | 370 | 7.2 |
| 6 | A | Product A | 3 | 10 | 370 | 7.2 |
| 7 | A | Product A | 6 | 10 | 370 | 7.2 |
| 8 | A | Product A | 10 | 10 | 370 | 7.2 |
| 9 | A | Product A | 1 | 25 | 370 | 7.2 |
| 10 | A | Product A | 3 | 25 | 370 | 7.2 |
| 11 | A | Product A | 6 | 25 | 370 | 7.2 |
| 12 | A | Product A | 10 | 25 | 370 | 7.2 |
| 13 | Com | Product A | 1 | 0 | 330 | 7.2 |
| 14 | Com | Product A | 3 | 0 | 330 | 7.2 |
| 15 | Com | Product A | 6 | 0 | 330 | 7.2 |
| 16 | Com | Product A | 10 | 0 | 330 | 7.2 |
| 17 | Com | Product A | 1 | 10 | 330 | 7.2 |
| 18 | Com | Product A | 3 | 10 | 330 | 7.2 |
| 19 | Com | Product A | 6 | 10 | 330 | 7.2 |
| 20 | Com | Product A | 10 | 10 | 330 | 7.2 |
| 21 | Com | Product A | 1 | 25 | 330 | 7.2 |
| 22 | Com | Product A | 3 | 25 | 330 | 7.2 |
| 23 | Com | Product A | 6 | 25 | 330 | 7.2 |
| 24 | Com | Product A | 10 | 25 | 330 | 7.2 |
| 25 | A | Product A | 3 | 0 | 370 | 7.2 |
| 26 | A | Product A | 6 | 15 | 370 | 7.2 |
| 27 | A | Product B | 3 | 0 | 370 | 7.2 |
| 28 | A | Product B | 6 | 15 | 370 | 7.2 |
| 29 | A | Product C | 3 | 0 | 370 | 7.2 |
| 30 | A | Product C | 6 | 15 | 370 | 7.2 |
| 31 | A | Product D | 3 | 0 | 370 | 7.2 |
| 32 | A | Product D | 6 | 15 | 370 | 7.2 |
| 33 | A | Product E | 3 | 0 | 370 | 7.2 |
| 34 | A | Product E | 6 | 15 | 370 | 7.2 |
| 35 | A | Product F | 3 | 0 | 370 | 7.2 |
| 36 | A | Product F | 6 | 15 | 370 | 7.2 |
| 37 | A | Product A | 3 | 0 | 370 | 7.0 |
| 38 | A | Product A | 6 | 15 | 370 | 7.0 |
| 39 | A | Product C | 3 | 0 | 370 | 7.0 |
| 40 | A | Product C | 6 | 15 | 370 | 7.0 |
| 41 | A | Product E | 3 | 0 | 370 | 7.0 |
| 42 | A | Product E | 6 | 15 | 370 | 7.0 |
| 43 | A | Product A | 3 | 0 | 300 | 7.2 |
| 44 | A | Product A | 6 | 15 | 300 | 7.2 |
| 45 | A | Product C | 3 | 0 | 300 | 7.2 |
| 46 | A | Product C | 6 | 15 | 300 | 7.2 |
| 47 | A | Product E | 3 | 0 | 300 | 7.2 |
| 48 | A | Product E | 6 | 15 | 300 | 7.2 |

The harvested and cell-separated cell culture supernatants were processed for CE-SDS analysis as described above to evaluate the levels of KiH main product and the abundance of HH side product (where relevant).

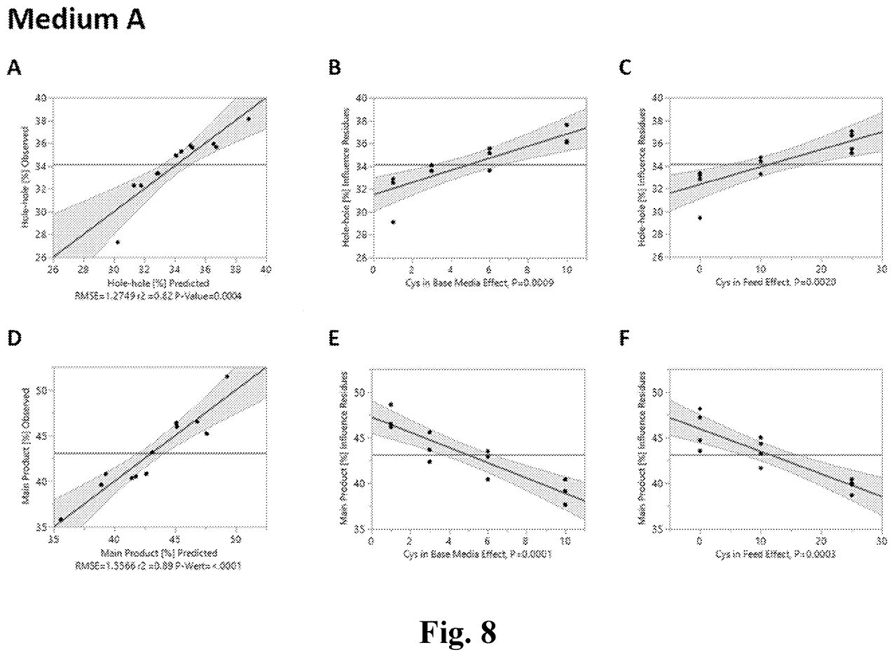

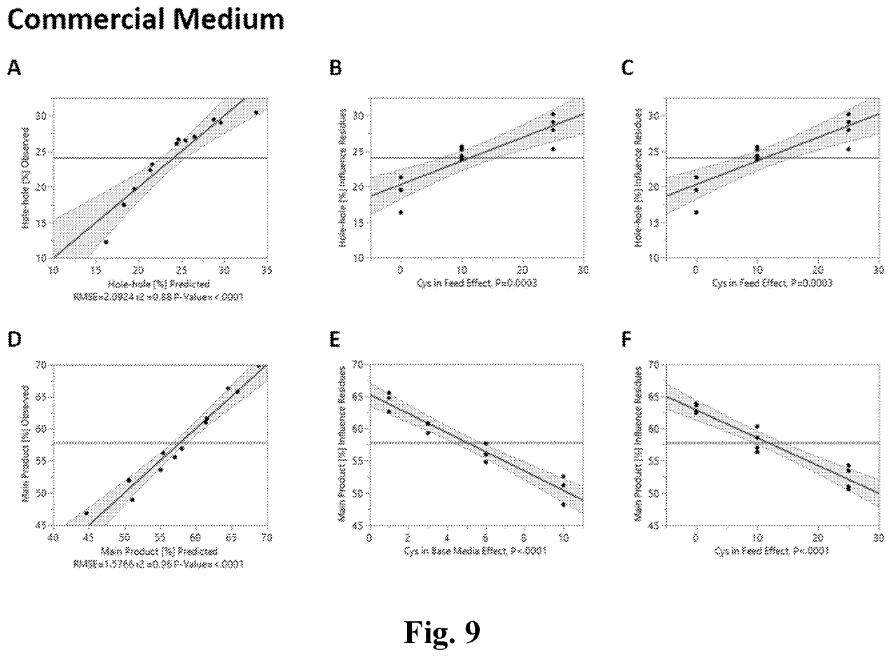

The experimentally acquired data can be described with good model quality for product A by statistically significant linear multi-regression models: Process A KiH main product: p-value<0.0001, RMSE: 1.5566 [%], r2: 0.89; HH Product: p-value 0.0004, RMSE: 1.2749 [%], r2: 0.82 (FIGS. 8A and 8D); Commercial: KiH main product: p-value<0.0001, RMSE: 1.5766 [%], r2: 0.96; HH product: p-value<0.0001, RMSE: 2.0924 [%], r2: 0.88 (FIGS. 9A and 9D).

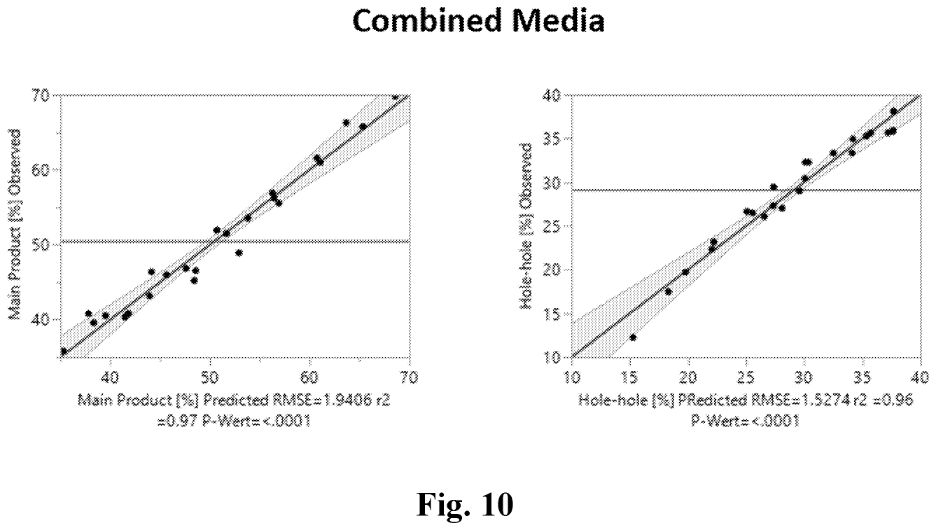

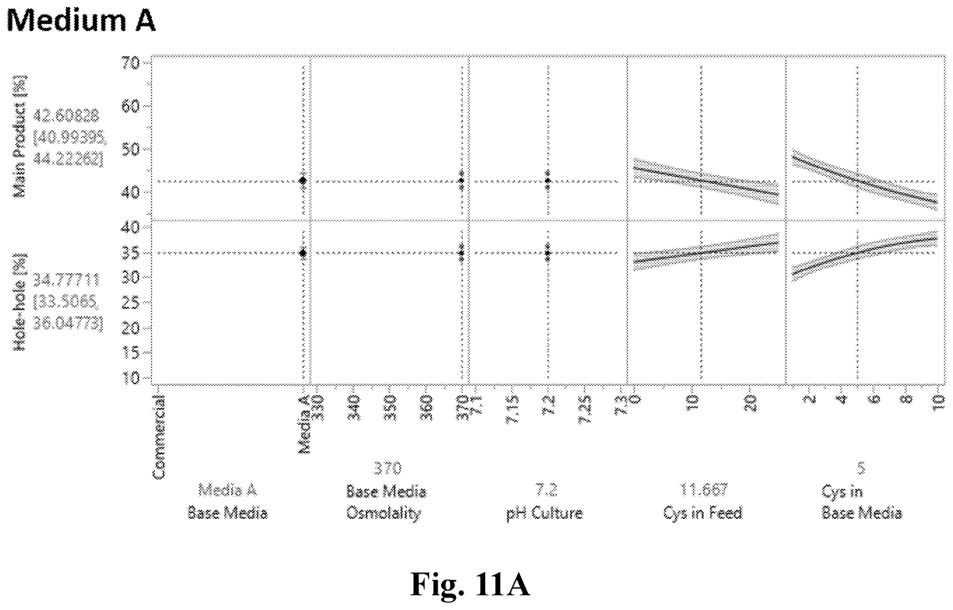

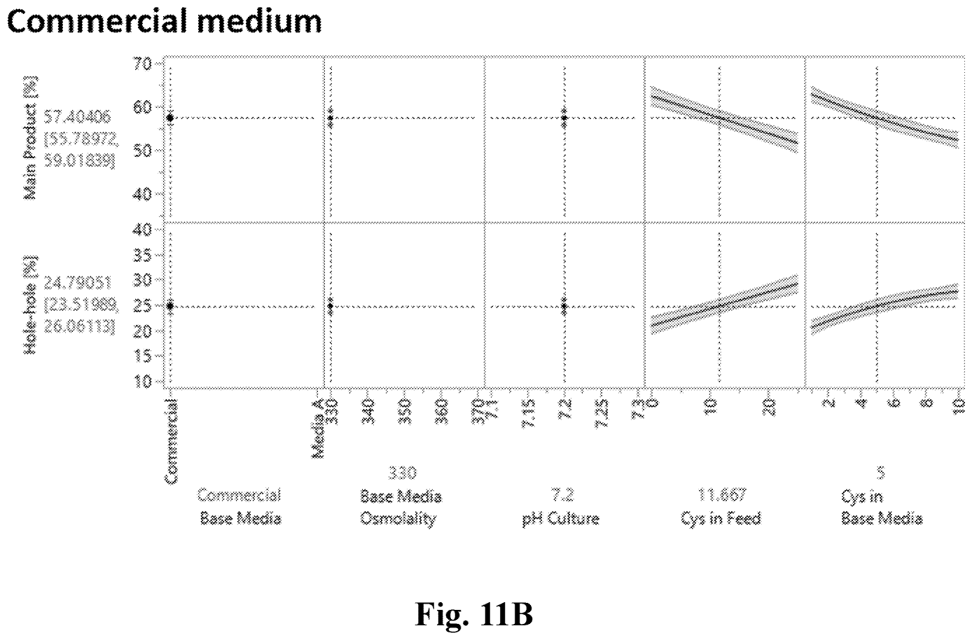

Both the cysteine level in the cell culture base medium and nutrient feed showed highly significant contributions to the observed effects. Cysteine, when alone in either the cell culture base medium or nutrient feed, or even more strongly in the cell culture base medium and nutrient feed, showed a dose-dependent negative effect on the KiH Main Product level abundance and a dose-dependent positive effect on the HH side product level for product A in separate linear regression models (shown by observed vs predicted plots and statistics). The effect was analysed by taking all data for product A into account to build a global model (FIG. 10) and for separated models, specific for Medium A (FIG. 8) and for Commercial Medium (FIG. 9). Both data analysis approaches result in the same conclusions: high levels of Cysteine, either alone in Base Medium, or in combination in the nutrient feed, lead to high levels of product A HH side product; whereas low levels of Cysteine, either alone in the base medium or in combination in the nutrient feed, lead to high levels of KiH target protein (as shown by prediction profiler plots, FIGS. 11A and 11B for Medium A and commercial medium respectively).

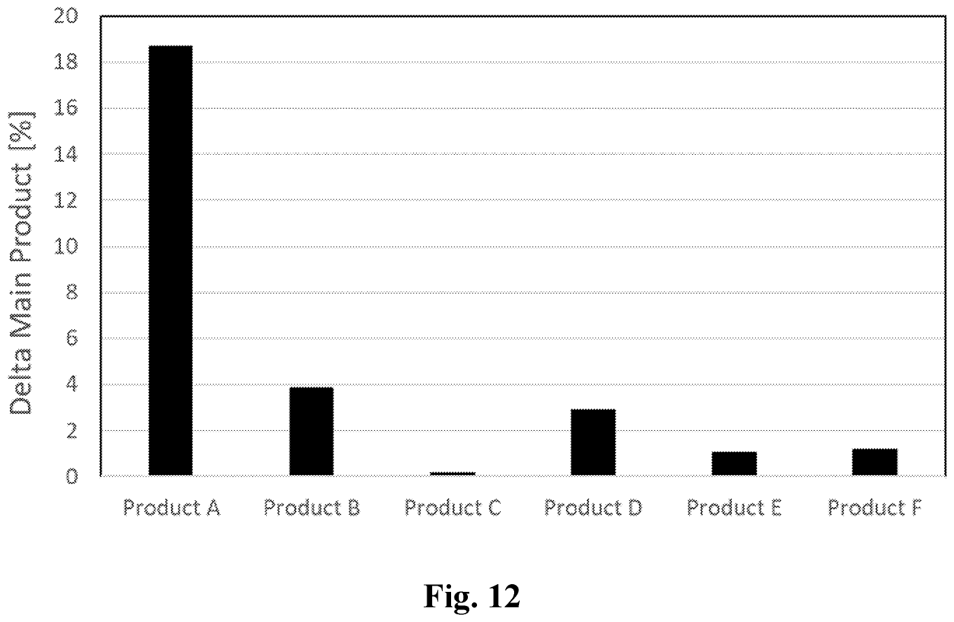

In the subsequent experiment series, experiments 25 to 48, two further 2+1 ectodomain fusion mAb (Product B and C), a 3+1 xmAb (Product D), a 2+1 TCB (T-cell bispecific) mAb (Product E) and a cytokine fusion mAb (Product F) (FIG. 5) were used and tested in cell culture experiments as described in Table 1 to prove the transferability of the effects to KiH products besides product A. A comparison between low Cysteine cell culture condition (3 mM Cysteine in base medium/0 mM Cysteine in nutrient feed) vs. high Cysteine cell culture condition (6 mM Cysteine in base medium/15 mM Cysteine in nutrient feed), showed higher KiH main product levels for all molecules (products A to F) in low Cysteine cell culture conditions, although, the delta of improvement levels varied depending on the starting product used (0.2% for Product C and 18.7% for product A) (Table 3, FIG. 12). The difference in KiH level [%] between ‘Low’ and ‘High’ Cysteine base media and feeding strategy was calculated as described below:

$$Difference in KiH Level [ \% ] = 100 \times ( KiH Main Product {Level}_{Low Cys} [ \% ] - KiH Main Product {Level}_{High Cys} [ \% ] ) / KiH Main Product {( Level )}_{Low Cys} [ \% ]$$

TABLE 2
Increase of KiH target protein levels by Cysteine base media and feeding strategy.

| | | KiH Main | | |
|---|---|---|---|---|
| | | Product | KiH Main Product | |
| | | Level_{Low Cys} [%] | Level_{High Cys} [%] | Difference in |
| | | (‘Low’ Cys | (‘High’ Cys | KiH Level |
| | | Strategy: 3 mM | Strategy: 6 mM Cys | [%] |
| | Molecule | Cys Base Medium / | Base Medium / 15 | (‘Low’/‘High’ |
| Product | Format | 0 mM Cys Feed) | mM Cys Feed) | Cys strategy) |
| Product A | 2 + 1 ectodomain | 48.03 | 39.04 | +18.71 |
| | fusion mAb | | | |
| Product B | 2 + 1 ectodomain | 66.18 | 63.61 | +3.88 |
| | fusion mAb | | | |
| Product C | 2 + 1 ectodomain | 90.16 | 90.00 | +0.18 |
| | fusion mAb | | | |
| Product D | 3 + 1 xmAb | 90.83 | 88.14 | +2.96 |
| Product E | 2 + 1 TCB (T-cell | 80.11 | 79.25 | +1.07 |
| | bispecific) mAb | | | |
| Product F | cytokine fusion | 92.18 | 91.08 | +1.19 |
| | mAb | | | |

### Example 3

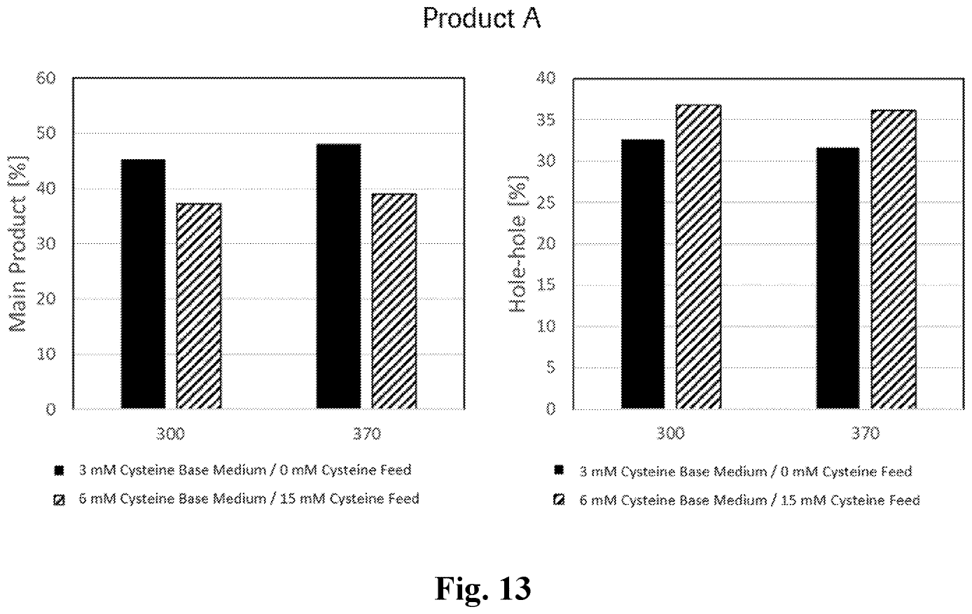

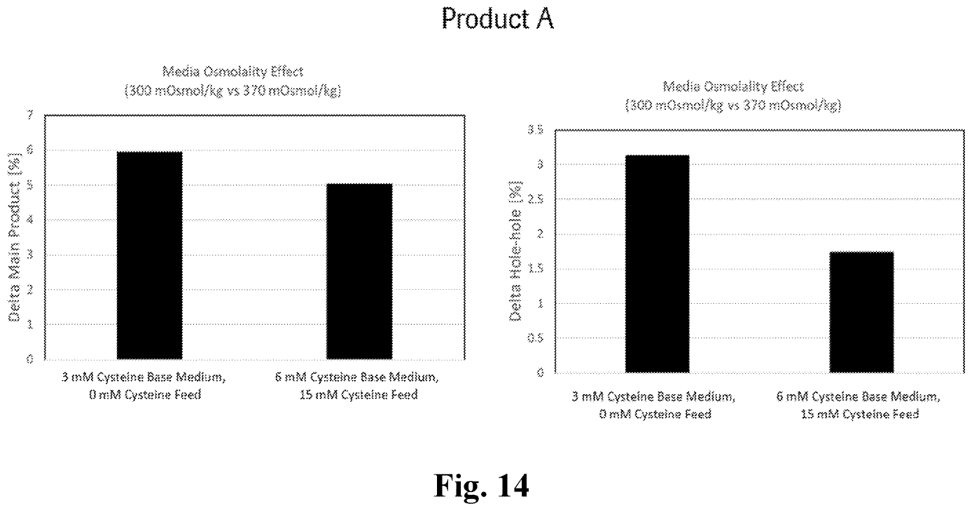

As chemicophysical parameters, cell culture pH and medium start osmolality were tested as putative modulators for KiH and HH modulation as described in Table 1. As described in Example 1, applying higher base medium start osmolality of 370 mOsmol/kg vs 300 mOsmol/kg lead to a modest but clear effect on KiH and HH levels of Product A; higher KiH levels and lower HH levels were detected (FIG. 13). The beneficial effect of base medium osmolality was stronger in low Cysteine cell culture conditions (3 mM Cysteine in base medium/0 mM Cysteine in nutrient feed), showing up to 6% improvement in KiH main product and up to 3% reduction in HH side product (FIG. 14).

Interestingly, no effect of cell culture pH on KiH and HH side product levels was observed.

In summary, a clear, unexpected modulation of KiH target protein quality level and abundance of unintended HH by-product by Cysteine in cell culture was observed. The effect was synergistic with cell culture base medium osmolality and independent of the tested KiH molecule.

1. A method for producing a molecule comprising a polypeptide complex formed by interaction between a first polypeptide comprising a CH3 region comprising a knob modification and a second polypeptide comprising a CH3 region comprising a hole modification, wherein the method comprises culturing cells comprising nucleic acid encoding the first and second polypeptides for the majority of the period of culture in cell culture medium comprising cysteine at a concentration of less than 6 mM.

2. The method according to claim 1, wherein the cell culture medium has an osmolality greater than 300 mOsmol/kg.

3. The method according to claim 1 or claim 2, wherein the method comprises seeding the cells in cell culture medium comprising cysteine at a concentration of less than 6 mM, optionally wherein the method comprises seeding the cells in cell culture medium comprising cysteine at a concentration of from 1 mM to 5 mM.

4. The method according to any one of claims 1 to 3, wherein the method comprises seeding the cells in cell culture medium comprising cysteine at a concentration of from 2.5 mM to 4.75 mM.

5. The method according to any one of claims 1 to 4, wherein the method comprises seeding the cells in cell culture medium having an osmolality greater than 300 mOsmol/kg, optionally wherein the method comprises seeding the cells in cell culture medium having an osmolality of from 320 mOsmol/kg to 420 mOsmol/kg.

6. The method according to any one of claims 1 to 5, wherein the method comprises seeding the cells in cell culture medium having an osmolality of about 370 mOsmol/kg.

7. The method according to any one of claims 1 to 6, wherein the period of culture is at least 3 days, optionally wherein the period of culture at least 7 days or at least 14 days.

8. The method according to any one of claims 1 to 7, wherein the cell culture medium has a pH of from 6.8 to 7.4, optionally wherein the cell culture medium has a pH of about 7.2.

9. The method according to any one of claims 1 to 8, wherein the first polypeptide or the second polypeptide comprises an amino acid sequence having at least 70% amino acid sequence identity to the amino acid sequence of a ligand for a costimulatory molecule.

10. The method according to any one of claims 1 to 9, wherein:
(i) the first polypeptide comprises an amino acid sequence having at least 70% amino acid sequence identity to the amino acid sequence of a ligand for a costimulatory molecule, and the second polypeptide comprises an amino acid sequence forming all or part of an antigen-binding moiety that binds to a target antigen; or
(ii) the first polypeptide comprises an amino acid sequence forming all or part of an antigen-binding moiety that binds to a target antigen, and the second polypeptide comprises an amino acid sequence having at least 70% amino acid sequence identity to the amino acid sequence of a ligand for a costimulatory molecule.
